SEQUENCE LISTING (2 sequences)

SEQ ID NO: 1 - protein, 611 aa, Homo sapiens
MWLAKACWSIQSEMPCIQAQYGTPAPSPGPRDHLASDPLTPEFIKPTMDLASPEAAPAAPTALPSFSTFMDGYTGEFDTFLYQLPGTVQPCSSASSSASSTSSSSATSPASASFKFEDFQVYGCYPGPLSGPVDEALSSSGSDYYGSPCSAPSPSTPSFQPPQLSPWDGSFGHFSPSQTYEGLRAWTEQLPKASGPPQPPAFFSFSPPTGPSPSLAQSPLKLFPSQATHQLGEGESYSMPTAFPGLAPTSPHLEGSGILDTPVTSTKARSGAPGGSEGRCAVCGDNASCQHYGVRTCEGCKGFFKRTVQKNAKYICLANKDCPVDKRRRNRCQFCRFQKCLAVGMVKEVVRTDSLKGRRGRLPSKPKQPPDASPANLLTSLVRAHLDSGPSTAKLDYSKFQELVLPHFGKEDAGDVQQFYDLLSGSLEVIRKWAEKIPGFAELSPADQDLLLESAFLELFILRLAYRSKPGEGKLIFCSGLVLHRLQCARGFGDWIDSILAFSRSLHSLLVDVPAFACLSALVLITDRHGLQEPRRVEELQNRIASCLKEHVAAVAGEPQPASCLSRLLGKLPELRTLCTQGLQRIFYLKLEDLVPPPPIIDKIFMDTLPF

SEQ ID NO: 2 - protein, 598 aa, Homo sapiens
MPCIQAQYGTPAPSPGPRDHLASDPLTPEFIKPTMDLASPEAAPAAPTALPSFSTFMDGYTGEFDTFLYQLPGTVQPCSSASSSASSTSSSSATSPASASFKFEDFQVYGCYPGPLSGPVDEALSSSGSDYYGSPCSAPSPSTPSFQPPQLSPWDGSFGHFSPSQTYEGLRAWTEQLPKASGPPQPPAFFSFSPPTGPSPSLAQSPLKLFPSQATHQLGEGESYSMPTAFPGLAPTSPHLEGSGILDTPVTSTKARSGAPGGSEGRCAVCGDNASCQHYGVRTCEGCKGFFKRTVQKNAKYICLANKDCPVDKRRRNRCQFCRFQKCLAVGMVKEVVRTDSLKGRRGRLPSKPKQPPDASPANLLTSLVRAHLDSGPSTAKLDYSKFQELVLPHFGKEDAGDVQQFYDLLSGSLEVIRKWAEKIPGFAELSPADQDLLLESAFLELFILRLAYRSKPGEGKLIFCSGLVLHRLQCARGFGDWIDSILAFSRSLHSLLVDVPAFACLSALVLITDRHGLQEPRRVEELQNRIASCLKEHVAAVAGEPQPASCLSRLLGKLPELRTLCTQGLQRIFYLKLEDLVPPPPIIDKIFMDTLPF

(19) United States
(12) Patent Application Publication
(10) Pub. No.: US 2013/0108602 A1
(43) Pub. Date: May 2, 2013

(54) METHODS AND USES OF NUR77 AND NUR77 AGONISTS TO MODULATE MACROPHAGES AND MONOCYTES, AND TREAT INFLAMMATION, INFLAMMATORY DISEASE AND CARDIOVASCULAR DISEASE

(71) Applicant: LA JOLLA INSTITUTE FOR ALLERGY AND IMMU, La Jolla, CA (US)

(72) Inventors: Catherine C. Hedrick, La Jolla, CA (US); Klaus Ley, La Jolla, CA (US); Richard Hanna, San Diego, CA (US); Iftach Shaked, San Diego, CA (US)

(73) Assignee: LA JOLLA INSTITUTE FOR ALLERGY AND IMMUNOLOGY, La Jolla, CA (US)

(21) Appl. No.: 13/646,183

(22) Filed: Oct. 5, 2012

Related U.S. Application Data
(63) Continuation of application No. PCT/US2011/031585, filed on Apr. 7, 2011.
(60) Provisional application No. 61/321,781, filed on Apr. 7, 2010.
(60) Provisional application No. 61/391,267, filed on Oct. 8, 2010.

(51) Int. Cl. A61K 38/17; A61K 31/404; A61K 31/235; A61K 35/14

(52) U.S. Cl. CPC A61K 38/1783; A61K 35/15; A61K 31/404; A61K 31/235
USPC 424/93.71; 514/21.2; 514/16.6; 514/1.7; 514/16.4; 514/15.4; 514/18.6; 514/17.7; 514/13.2; 514/18.3; 514/415; 514/545

(57) ABSTRACT

Methods of decreasing, reducing, inhibiting, suppressing, limiting or controlling an undesirable or aberrant immune response, immune disorder, inflammatory response, or inflammation in a subject; decreasing, reducing, inhibiting, suppressing, limiting or controlling an autoimmune response, disorder or disease in a subject; and decreasing, reducing, inhibiting, suppressing, limiting or controlling an adverse cardiovascular event or cardiovascular disease in a subject, are provided. Methods include, for example, administering a Nur77 polypeptide or subsequence thereof, a Nur77 agonist, or CD14⁺ CD16⁺ monocytes or CD14^{dim}CD16⁺ (CD115⁺CD11b⁺GR1⁻ (Ly6C−)) monocytes or macrophages to a subject to decrease, reduce, inhibit, suppress, limit or control the underlying condition or an adverse symptom or pathology of the condition.

### RELATED APPLICATIONS

This application claims the benefit of priority of application Ser. No. 61/321,781 filed Apr. 7, 2010, and application Ser. No. 61/391,267 filed Oct. 8, 2010, both of which applications are expressly incorporated herein by reference in their entirety.

### GOVERNMENT SUPPORT

This work was supported in part by Grants HL058108 and HL079621 from the National Institutes of Health. The government has certain rights in the invention.

### INTRODUCTION

Nur77, along with Nurr1 and NOR-1, constitute the NR4A subfamily of orphan nuclear receptors in the steroid/thyroid receptor family (Martinez-Gonzalez et al., 2005). Nur77 has been implicated in the differentiation, proliferation, apoptosis, and survival of many different cell types. Nur77 was originally identified as a growth factor inducible gene, and is often over-expressed in a variety of cancer cells including lung, prostate, breast and colon cancers (Lim et al., Oncogene 1:263 (1987); Hazel et al., Proc Natl Acad Sci USA 85:8444 (1988); Milbrandt, Neuron 1:183 (1988); Chang et al., J Steroid Biochem 34:391 (1989); Uemura and Chang, Endocrinology 139:2329 (1998); Wu et al., Oncogene 21:3925 (2002); Jeong et al., Ann N Y Acad Sci 1010:171 (2003); Holla et al., J Biol Chem 281:2676 (2005); Maddika et al., J Cell Sci 118:4485 (2005)). A number of growth factors and mitogens can potently and rapidly induce the expression of Nur77, suggesting an anti-apoptotic role of Nur77 in mediating growth of cancer cells. Contrarily, Nur77 has been implicated in programmed cell death of T and B lymphocytes (Rajpal et al., EMBO J. 22:6526 (2003); Lee et al., Proc Natl Acad Sci USA 99:11878 (2002)). Nur77 is rapidly induced by T-cell receptor signals, and dominant negative Nur77 inhibits clonal deletion of developing T cells, indicating a role for Nur77 and its family members in thymocyte apoptosis (Zhou et al., J Exp Med 183:1879 (1996); Woronicz et al., Nature 367:277 (2004); Cho et al., J Immunol 170:10 (2003)). Nur77 mRNA is also rapidly (<1 hour) induced in macrophages in response to a variety of inflammatory stimuli, including LPS, cytokines, and oxidized lipids (Pei et al., J Biol Chem 280:29256 (2005)).

Structural and functional studies of Nur77 and other NR4A family members suggest that these nuclear receptor family members do not need to bind small-molecule ligands to be activated (Baker et al., Cell 113:731 (2003); Wang et al., Nature 423:555 (2003)), and instead are regulated by posttranslational modifications such as phosphorylation (Fahrner et al., Mol Cell Biol 10:6454 (1990)). Acting as a transcription factor, Nur77 can directly bind specific DNA response elements alone or can heterodimerize with the retinoid X receptor (RXR) (Wilson et al., Science 252:1296 (1991); Wallen-Mackenzie et al., Genes Dev 17:3036 (2003)). Nur77 binding sites have been identified on the IKBα promoter and have been suggested as a means for regulating NFKB inflammatory signaling (You et al., Circ Res 104:742 (2009)). In response to apoptotic stimuli, Nur77 may dimerize with RXR, and translocate from the nucleus to the cytoplasm where it can target mitochondria to induce cytochrome c release and apoptosis (Li et al., Science 289:1159 (2000)). Consistent with its role as an apoptotic mediator in T and B cells, Nur77 can target mitochondrial function through its interaction with Bcl-2, converting Bcl-2 from an anti-apoptotic to a pro-apoptotic molecule (Lin et al., Cell 116:527 (2004); Thompson and Winoto, J Exp Med 205:1029 (2008)).

Little is known about the exact functions of Nur77 in monocyte biology. Nur77 is expressed in human atherosclerotic lesion macrophages, and reduces human macrophage lipid loading and inflammatory responses in atherosclerotic plaques (Bonta et al., Arterioscler Thromb Vasc Biol 26:2288 (2006); Arkenbout et al., Circulation 106:1530 (2002)). Nur77 expression also inhibits macrophage accumulation and vascular remodeling in mice (Bonta et al., Cardiovasc Res 87:561 (2010)). Conneely and colleagues generated a NOR-1−/− Nur77−/− double knockout mouse and observed development of acute myeloid leukemia in the mice, with abnormal expansion of myeloid progenitor cells (Mullican et al., Nat Med 13:730 (2007)). However, mice deficient in either Nur77 or NOR-1 have relatively subtle abnormalities and lack overt defects in general physiology, consistent with the idea that NR4A family members have some functional redundancy (Lee et al., Science 269:532 (1995)). Interestingly, Nurr1 (also know as NR4A2), a related NR4A family member, has recently demonstrated a regulatory role in maintaining hematopoietic stem cell quiescence (Sinn et al., Nat Cell Biol 12:1213 (2010)).

In mice and humans, at least two distinct blood monocyte subsets exist which can be identified as being CD11b+ and CD115+. In mice, Ly6C+, CCR2+, CX3CR1^{lo}, CD62L+ monocytes are inflammatory and migrate to injured or infected sites, and Ly6C−, CCR2−, CX3CR1^{high} and CD62L− monocytes patrol the resting vasculature and participate in the resolution of inflammation (Randolph et al., Curr Opin Lipidol 19:462 (2008); Tacke et al., J Exp Med 203:583 (2006)). Mouse Ly6C+ monocytes correspond to CD14+CD16− inflammatory monocytes in humans, and Ly6C− mouse monocytes serve as counterparts to CD14^{dim} CD16+ and CD14+CD16+ human patrolling monocytes (Cros et al., Immunity 33:375 (2010); Randolph, J Thromb Haemost 7:28 (2009)). Ly6C+ monocytes are selectively recruited to inflamed or infected tissues and lymph nodes (Randolph et al., Curr Opin Lipidol 19:462 (2008); Geissmann et al., Immunity 19:71 (2003); Palframan et al., J Exp Med 194:1361 (2001)). Ly6C− monocytes are believed to be monocytes that don't readily migrate in response to inflammation; rather they tend to patrol the resting vasculature, populate normal or inflamed sites, and participate in the resolution of inflammation (Nahrendorf et al., J Exp Med 204:3037 (2007); Auffray et al., Science 317:666 (2007); Geissmann et al. Immunity 19:71 (2003); Tacke et al., J Clin Invest 117:185 (2007)).

Monocyte subsets arise from a common macrophage dendritic precursor (MDP) in the bone marrow (Auffray et al., J Exp Med 206:595 (2009)). However, the details of the differentiation steps and intermediaries between MDPs and monocytes subsets are unclear. Adoptive transfer studies demonstrate that Ly6C+ monocytes can down-regulate Ly6C expression, and move between blood and bone marrow (Arnold et al., J Exp Med 204:1057 (2007); Varol et al., J Exp Med 204:171 (2007); Yrlid et al., J Immunol 176:4155 (2006)), but whether these are functionally equivalent to patrolling Ly6C− monocytes has been questioned (Geissmann et al., Science 327:656 (2010)). In contrast, other groups have demonstrated that distinct populations of monocytes are recruited to inflammation/injury sites (Auffray et al., Science 317:666 (2007); Nahrendorf et al., J Exp Med 204:3037 (2007); Landsman et al., J Immunol 178:2000 (2007)). A number of transcription factors including PU.1, JunB, CEBPα/β, and IRF8 have important roles in myeloid lineage differentiation, but the specific factors that drive differentiation of Ly6C− monocytes are unknown (Auffray et al., Annu Rev Immunol 27:669 (2009)). As disclosed herein, Nur77 has a functional role in the differentiation and maintenance of Ly6C⁻ monocytes in bone marrow, indicating that Nur77 is a critical transcription factor driving monocyte development and effector function in vivo. Accordingly, Nur77 and Nur77 agonists are useful to selectively manipulate monocyte subsets for therapeutic, preventive and other purposes.

### SUMMARY

The orphan nuclear receptor Nur77 (also known as NR4A1 or NGFI-B) has emerged as an important regulator of macrophage function. As disclosed herein, a monocyte subset important for patrolling blood vessels is absent in mice lacking Nur77 (Nur77−/− or Nr4a1−/− mice). CD11b+, CD115+, Ly6C− monocytes, which express relatively high levels of Nur77 and are comparable to CD14^{int} CD16+ monocytes in humans, are largely absent from the blood, bone marrow, and spleen of Nur77−/− mice. Normal levels of myeloid progenitor cells are present in the marrow of Nur77−/− mice, suggesting that the defect occurs in the bone marrow at the later stages of monocyte development and maturation, more specifically after the macrophage dendritic precursor (MDP) stage. Moreover, the defect is cell-intrinsic, as wild-type mice receiving bone marrow from Nur77−/− mice developed the monocyte defect, whereas Nur77−/− mice receiving wild-type bone marrow were rescued, and developed normal levels of the Ly6C− monocyte subset. The few Ly6C− monocytes that remained in the bone marrow of Nur77−/− mice appeared morphologically similar to Ly6C⁺ monocytes. However, Ly6C− monocytes from Nur77−/− mice were undergoing apoptosis, and appeared to be arrested in the S phase of the cell cycle. These Ly6C−Nur77−/− monocytes also expressed reduced levels of CX3CR1 and LFA-1, indicating that they were not homing normally to the endothelium. Ly6C− monocytes from Nur77−/− mice exhibited increased NFkB activity, and resulting resident macrophage populations are more inflammatory. These and other results disclosed herein demonstrate that Nur77 is required for normal development and survival of patrolling Ly6C− monocytes, and that Nur77 expression in patrolling monocyte populations is important for suppressing inflammation.

In accordance with the invention, methods and uses for decreasing, reducing, inhibiting, suppressing, limiting or controlling an undesirable or aberrant immune response, immune disorder, inflammatory response, or inflammation in a subject; methods and uses for decreasing, reducing, inhibiting, suppressing, limiting or controlling an autoimmune response, disorder or disease in a subject; and methods and uses for decreasing, reducing, inhibiting, suppressing, limiting or controlling an adverse cardiovascular event or cardiovascular disease in a subject are provided. In one embodiment, a method includes administering a Nur77 polypeptide or subsequence thereof, a Nur77 agonist, or CD14⁺ CD16⁺ monocytes and/or CD14^{dim}CD16⁺ (CD115⁺CD11b⁺GR1⁻ (Ly6C−)) monocytes or macrophages to a subject in an amount to decrease, reduce, inhibit, suppress, limit or control the undesirable or aberrant immune response, immune disorder, inflammatory response, or inflammation in the subject. In one embodiment, a method includes administering a Nur77 polypeptide or subsequence thereof, a Nur77 agonist, or CD14⁺ CD16⁺ monocytes and/or CD14^{dim}CD16⁺ (CD115⁺CD11b⁺GR1⁻ (Ly6C−)) monocytes or macrophages to a subject in an amount to decrease, reduce, inhibit, suppress, limit or control an autoimmune response, disorder or disease in the subject. In one embodiment, a method of decreasing, reducing, inhibiting, suppressing, limiting or controlling an adverse cardiovascular event or cardiovascular disease in a subject includes administering a Nur77 polypeptide or subsequence thereof, a Nur77 agonist, or CD14⁺ CD16⁺ monocytes and/or CD14^{dim}CD16⁺ (CD115⁺CD11b⁺GR1⁻ (Ly6C−)) monocytes or macrophages to a subject in an amount to decrease, reduce, inhibit, suppress, limit or control the adverse cardiovascular event or cardiovascular disease in the subject.

Methods and uses for the invention include adverse cardiovascular events and cardiovascular diseases. Non-limiting examples include coronary artery or heart disease, atherosclerosis, peripheral artery disease, cerebrovascular disease, renal artery disease, stroke, myocardial infarction (heart attack), ischemic heart failure, transient ischemic attack and brain trauma, and elevated blood cholesterol.

Methods and uses of the invention include decreasing, reducing, inhibiting, suppressing, limiting or controling an adverse symptom of an undesirable or aberrant immune response, immune disorder, inflammatory response, or inflammation, an adverse symptom of the autoimmune response, disorder or disease, or an adverse symptom of the cardiovascular event or cardiovascular disease in the subject. Non-limiting examples include swelling, pain, rash, headache, fever, nausea, diarrhea, bloat, lethargy, skeletal joint stiffness, stroke, pain, paralysis, vision impairment, other sense impairment, or tissue or cell damage.

Methods and uses of the invention include treatment of any affected cell, tissue or organ, locally, regionally, or systemically. Non-limiting examples include an epidermal or mucosal tissue, gut, bowel, pancreas, thymus, liver, kidney, muscle, central or peripheral nerves, spleen, skin, a skeletal joint, blood vessel, or a cardio-pulmonary tissue or organ.

Methods and uses of the invention include treatment of any immune response, immune disorder, inflammatory response, inflammation, or autoimmune response, disorder or disease, or adverse cardiovascular event or cardiovascular disease, either chronic or acute. Such methods and uses can be practiced on a subject such as a subject in need of treatment, for example, an affected subject or a subject at risk of any immune response, immune disorder, inflammatory response, inflammation, or autoimmune response, disorder or disease, or adverse cardiovascular event or cardiovascular disease, chronic or acute.

Methods and uses of the invention include administration or use of a Nur77 polypeptide or subsequence thereof, or a Nur77 agonist. Such Nur77 polypeptides and subsequences thereof, and Nur77 agonists include those that stimulate, promote, increase or induce CD14⁺ CD16⁺ or CD14^{dim} CD16⁺ monocyte or macrophage cell production, development, survival, proliferation, differentiation or activity.

In accordance with the invention, also provided are methods and uses for stimulating, promoting, increasing or inducing CD14⁺ CD16⁺ and/or CD14^{dim} CD16⁺ monocyte or macrophage cell production, development, survival, proliferation, differentiation or activity. In one embodiment, a method includes administering a Nur77 polypeptide or a subsequence thereof, or a Nur77 agonist, to a subject in an amount that stimulates, promotes, increases or induces CD14⁺ CD16⁺ or CD14^{dim} CD16⁺ monocyte or macrophage cell production, development, survival, proliferation, differentiation or activity.

In accordance with the invention, further provided are methods and uses for stimulating, promoting, increasing or inducing CD14⁺ CD16⁺ and/or CD14^{dim} CD16⁺ monocyte or macrophage cell production, development, survival, proliferation, differentiation or activity. In one embodiment, a method includes contacting monocyte dendritic precursor cells with a Nur77 polypeptide or a subsequence thereof, or a Nur77 agonist, in an amount that stimulates, promotes, increases or induces CD14⁺ CD16⁺ or CD14^{dim} CD16⁺ monocyte or macrophage cell production, development, survival, proliferation, differentiation or activity.

In accordance with the methods and uses of the invention, Nur77 polypeptide or subsequence thereof includes full length Nur77 and subsequences of full length Nur77 polypeptide. Also in accordance with the methods and uses of the invention, Nur77 polypeptides and subsequences thereof and Nur77 agonists include mammalian (e.g., human) Nur77 polypeptides and subsequences thereof and Nur77 agonists.

Non-limiting Nur77 polypeptides and subsequences thereof include full length and subsequences of a human protein sequence, and polymorphisms thereof, set forth as:

Human NR4A1 protein sequence (Homo sapiens nuclear receptor subfamily 4, group A, member 1 (NR4A1):

MWLAKACWSIQSEMPCIQAQYGTPAPSPGPRDHLASDPLTPEFIKPTMDL
ASPEAAPAAPTALPSFSTFMDGYTGEFDTFLYQLPGTVQPCSSASSSASS
TSSSSATSPASASFKFEDFQVYGCYPGPLSGPVDEALSSSGSDYYGSPCS
APSPSTPSFQPPQLSPWDGSFGHFSPSQTYEGLRAWTEQLPKASGPPQPP
AFFSFSPPTGPSPSLAQSPLKLFPSQATHQLGEGESYSMPTAFPGLAPTS
PHLEGSGILDTPVTSTKARSGAPGGSEGRCAVCGDNASCQHYGVRTCEGC
KGFFKRTVQKNAKYICLANKDCPVDKRRRNRCQFCRFQKCLAVGMVKEVV
RTDSLKGRRGRLPSKPKQPPDASPANLLTSLVRAHLDSGPSTAKLDYSKF
QELVLPHFGKEDAGDVQQFYDLLSGSLEVIRKWAEKIPGFAELSPADQDL
LLESAFLELFILRLAYRSKPGEGKLIFCSGLVLHRLQCARGFGDWIDSIL
AFSRSLHSLLVDVPAFACLSALVLITDRHGLQEPRRVEELQNRIASCLKE
HVAAVAGEPQPASCLSRLLGKLPELRTLCTQGLQRIFYLKLEDLVPPPPI
IDKIFMDTLPF;

and
Homo sapiens nuclear receptor subfamily 4, group A, member 1 (NR4A1)

MPCIQAQYGTPAPSPGPRDHLASDPLTPEFIKPTMDLASPEAAPAAPTAL
PSFSTFMDGYTGEFDTFLYQLPGTVQPCSSASSSASSTSSSSATSPASAS
FKFEDFQVYGCYPGPLSGPVDEALSSSGSDYYGSPCSAPSPSTPSFQPPQ
LSPWDGSFGHFSPSQTYEGLRAWTEQLPKASGPPQPPAFFSFSPPTGPSP
SLAQSPLKLFPSQATHQLGEGESYSMPTAFPGLAPTSPHLEGSGILDTPV
TSTKARSGAPGGSEGRCAVCGDNASCQHYGVRTCEGCKGFFKRTVQKNAK
YICLANKDCPVDKRRRNRCQFCRFQKCLAVGMVKEVVRTDSLKGRRGRLP
SKPKQPPDASPANLLTSLVRAHLDSGPSTAKLDYSKFQELVLPHFGKEDA
GDVQQFYDLLSGSLEVIRKWAEKIPGFAELSPADQDLLLESAFLELFILR
LAYRSKPGEGKLIFCSGLVLHRLQCARGFGDWIDSILAFSRSLHSLLVDV
PAFACLSALVLITDRHGLQEPRRVEELQNRIASCLKEHVAAVAGEPQPAS
CLSRLLGKLPELRTLCTQGLQRIFYLKLEDLVPPPPIIDKIFMDTLPF.

Non-limiting Nur77 polymorphisms of the invention include an amino acid substitution at one or more amino acid positions 26, 36, 74, 137, 262, or 400 of Nur77. In particular aspects, a polymorphism is a change of one or more of: a (L) Leu to (V) Val at position 26; a (D) Asp to (G) Gly at position 36; a (T) Thr to (I) Ile at position 74; an (S) Ser to (L) Leu at position 137; a (G) Gly to (R) Arg at position 262; a (G) Gly to (A) Ala at position 262; or a (A) Ala to (D) Asp at position 400 of Nur77.

In accordance with the methods and uses of the invention, Nur77 polypeptides and subsequences thereof and Nur77 agonists include modified or variant forms. In particular aspects, a Nur77 polypeptide, subsequence thereof or Nur77 agonist includes a fusion polypeptide, or a chimeric polypeptide. In additional particular aspects, a Nur77 polypeptide, subsequence thereof or Nur77 agonist is fused to a tag or label, or an immunoglobulin polypeptide (e.g., IgG, IgA, IgM, IgE or IgD).

In accordance with the methods and uses of the invention, Nur77 agonists include agonists that increase expression or activity of Nur77. In particular aspects, Nur77 agonists include small molecules. Non-limiting examples of such agonists and small moeclues include: 9-cis-retinoic acid; 1-di(3-indolyl)-1-(4-X-phenyl)methanes; etoposide; 5,8-diacetoxyl-6-(1′-acetoxy-4′-methyl-3′-pentenyl)-1,4-naphthaquinones; 12-O-tetradecanoylphorbol-13-acetate; diindolylmethane analogs (C-DIMs); 6-mercaptopurine; panobinostat ((2E)-N-hydroxy-3-[4-({[2-(2-methyl-1H-indol-3-yl)ethyl]amino}methyl)phenyl]acrylamide); octaketide Cytosporone B (Csn-B); and derivatives thereof.

In accordance with the methods and uses of the invention, Nur77 polypeptides, subsequences thereof, Nur77 agonists and small molecules, and CD14⁺ CD16⁺ and/or CD14^{dim} CD16⁺ monocytes can be administered (delivered) by any appropriate means. In particular aspects, Nur77 polypeptides, subsequences thereof, Nur77 agonists and small molecules, and CD14⁺ CD16⁺ and/or CD14^{dim} CD16⁺ monocytes are delivered (administered) by injection, infusion, catheter, enema, intravenously, intraarterially, orally, intramuscularly, intraperitoneally, intradermally, subcutaneously, intracavity, intrarectally, intracranially, topically, transdermally, optically, parenterally, or transmucosally.

Nur77 polypeptides, subsequences thereof, Nur77 agonists and small molecules, and CD14⁺ CD16⁺ and/or CD14^{dim} CD16⁺ monocytes can be administered (delivered) for any period of time desirable (e.g., 60 minutes or less, for a period of 30 minutes or less, for a period of 15 minutes or less, or for a period of 5 minutes or less, or for a period of 1 minute or less). Nur77 polypeptides, subsequences thereof, Nur77 agonists and small molecules, and CD14⁺ CD16⁺ and/or CD14^{dim} CD16⁺ monocytes can be administered (delivered) at the time of or during a symptom or shortly thereafter, for example, within 72, 48, 36, 24, 12, 8, 6, 4, 2 or 1 hours (or less 1-15, 15020, or 30-60 minutes) of a symptom of an undesirable or aberrant immune response, immune disorder, inflammatory response, or inflammation, or an autoimmune response, disorder or disease, or adverse cardiovascular event or cardiovascular disease.

Nur77 polypeptides, subsequences thereof, Nur77 agonists and small molecules, and CD14⁺ CD16⁺ and/or CD14^{dim} CD16⁺ monocytes are appropriate for treatment of various immune disorders, inflammatory responses, inflammation, autoimmune responses, disorders and diseases. Non-limiting examples include rheumatoid arthritis, juvenile rheumatoid arthritis, osteoarthritis, psoriatic arthritis, multiple sclerosis (MS), encephalomyelitis, myasthenia gravis, systemic lupus erythematosus (SLE), asthma, allergic asthma, autoimmune thyroiditis, atopic dermatitis, eczematous dermatitis, psoriasis, Sjögren's Syndrome, Crohn's disease, aphthous ulcer, iritis, conjunctivitis, keratoconjunctivitis, ulcerative colitis (UC), inflammatory bowel disease (IBD), cutaneous lupus erythematosus, scleroderma, vaginitis, proctitis, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, interstitial lung fibrosis, Hashimoto's thyroiditis, autoimmune polyglandular syndrome, insulin-dependent diabetes mellitus, insulin-resistant diabetes mellitus, immune-mediated infertility, autoimmune Addison's disease, pemphigus vulgaris, pemphigus foliaceus, dermatitis herpetiformis, autoimmune alopecia, vitiligo, autoimmune hemolytic anemia, autoimmune thrombocytopenic purpura, pernicious anemia, Guillain-Barre syndrome, stiff-man syndrome, acute rheumatic fever, sympathetic ophthalmia, Goodpasture's syndrome, systemic necrotizing vasculitis, antiphospholipid syndrome or an allergy, Behcet's disease, severe combined immunodeficiency (SCID), recombinase activating gene (RAG 1/2) deficiency, adenosine deaminase (ADA) deficiency, interleukin receptor common γ chain (γ_{c}) deficiency, Janus-associated kinase 3 (JAK3) deficiency and reticular dysgenesis; primary T cell immunodeficiency such as DiGeorge syndrome, Nude syndrome, T cell receptor deficiency, MHC class II deficiency, TAP-2 deficiency (MHC class I deficiency), ZAP70 tyrosine kinase deficiency and purine nucleotide phosphorylase (PNP) deficiency, antibody deficiencies, X-linked agammaglobulinemia (Bruton's tyrosine kinase deficiency), autosomal recessive agammaglobulinemia, Mu heavy chain deficiency, surrogate light chain (γ5/14.1) deficiency, Hyper-IgM syndrome: X-linked (CD40 ligand deficiency) or non-X-linked, Ig heavy chain gene deletion, IgA deficiency, deficiency of IgG subclasses (with or without IgA deficiency), common variable immunodeficiency (CVID), antibody deficiency with normal immunoglobulins; transient hypogammaglobulinemia of infancy, interferon γ receptor (IFNGR1, IFNGR2) deficiency, interleukin 12 or interleukin 12 receptor deficiency, immunodeficiency with thymoma, Wiskott-Aldrich syndrome (WAS protein deficiency), ataxia telangiectasia (ATM deficiency), X-linked lymphoproliferative syndrome (SH2D1A/SAP deficiency), and hyper IgE syndrome.

In accordance with the invention, additionally provided are Nur77 polypeptides and subsequences thereof compositions including pharmaceutical compositions and formulations including Nur77 polypeptides or subsequences thereof. Such compositions and formulations include mammalian (e.g., human) Nur77, full length and subsequences of Nur77, and Nur77 variants and modified forms including polymorphisms of Nur77 as set forth herein or known to the skilled artisan. Invention compositions and formulations include those developed for a particular invention method, treatment or use, or administration or delivery means or protocol as set forth herein. Such compositions and formulations also include combinations of Nur77 polypeptide or subsequence thereof, or a Nur77 agonist, or CD14⁺ CD16⁺ monocytes and/or CD14^{dim}CD16⁺ (CD115⁺CD11b⁺GR1⁻ (LyC6−)) monocytes or macrophages with each other, as well as in combination with a drug or agent or treatment. In particular aspects, a drug, agent or treatment is for an aberrant immune response, immune disorder, inflammatory response, inflammation, an autoimmune response, disorder or disease, or an adverse cardiovascular event or cardiovascular disease.

### DESCRIPTION OF THE DRAWINGS

FIGS. 1A-1C show: A. Gating strategy for identification of monocyte populations by flow cytometry of mouse spleen (top). Live, single, Lin− (CD3e, CD19, CD49b, Ly6G) cells were plotted for CD115 and CD11b expression. CD115+, CD11b+ monocytes were then subdivided based on their + or − expression of Ly6C. B. The two monocyte populations (Ly6C+ and −) were further examined for expression of a variety of surface markers by flow cytometry. C. The two monocyte populations varied in their expression of Ly6C, CD62L, CD11c, CCR2 and CX3CR1.

FIGS. 2A-2E show: A. Representative flow cytometric scatter plot of CD11b⁺, CD115+ monocyte populations further gated on Ly6C+ or − expression in the spleen of global Nur77−/− mice or BL6 control mice (percentage of total population displayed on plot; red box highlights Ly6C− monocyte population). B. Quantification of the number of total monocytes/spleen (left), the number of Ly6C+ and − monocytes/spleen (center), and the percent of Ly6C+ and − monocytes/spleen of all live cells (right) analyzed by flow cytometry (Mean+/−SE *=p<0.05 n=10). Quantification of Ly6C+ and − monocytes populations in blood and bone marrow of global Nur77−/− mice and BL6 control mice analyzed by flow cytometry (bottom). (Mean+/−SE *=p<0.05 n=10) C. Quantification of hematopoietic stem cells (HSC), common myeloid precursors (CMP) and macrophage dendritic precursor (MDP) cell population in the bone marrow of global Nur77−/− mice and BL6 control mice analyzed by flow cytometry (left). (Mean+/−SE *=p<0.05 n=10) Quantification of other major hematopoietic cell populations found in the blood of global Nur77−/− mice and BL6 control mice analyzed by flow cytometry (right) (Mean+/−SE *=p<0.05 n=10) D. CD115+ CD11b+Ly6C+ and − monocytes were isolated from Nur77−/− and BL6 control bone marrow by FACS. Forward and side scatter (FSC and SSC) values are displayed in the upper left corner as mean+/−SE. (scale bar=10 μm) E. Relative mRNA expression of NR4A1 family members Nur77, Nurr1, and Nor-1 in bone marrow of FACS sorted Ly6C⁺, Ly6C−, MDP and CMP populations analyzed by qRT-PCR (left; Mean+/−SE *=p<0.05 n=6; expressed as a percentage of Ly6C+ monocyte transcript). Nur77 protein in bone marrow Ly6C+ and Ly6C− monocyte populations, and CD11b− non-myeloid cells measured by flow cytometric intracellular staining with a Nur77 specific antibody (right). Isolated monocyte populations were determined to be over 95% pure by Cytospin preps of sorted cells stained with HEMA3 dye. Live cells with low side scatter and Lin−(CD3e, CD19, CD49b, Ly6G) were plotted for CD115+ and CD11b+ expression and then gated for Ly6C+/− expression. (Mean+/−SE *=p<0.05 n=5) Results are expressed as % of live cells unless otherwise noted.

FIGS. 3A-3D show: A. Representative analysis of Ly6C+ and − monocyte population in the blood of Nur77−/− mice recipients of Nur77−/− or BL6 control transplanted whole bone marrow. B. Quantification of monocyte populations in blood from whole bone marrow transplants of either BL6 or Nur77−/− donor bone marrow into lethally irradiated BL6 or Nur77−/− recipient mice. C. Mixed chimera transplants of whole bone marrow 1:1 (Nur77−/− CD45.2: BL6 CD45.1) mixed dopers into BL6 CD45.1 recipients D. MDP cells were isolated by FACS from Nur77−/− or BL6 control bone marrow and mixed 1:1 (Nur77−/− CD45.2: BL6 CD45.1), and then reconstituted for seven days into lethally irradiated mice before analysis. For whole bone marrow transplants, mice were irradiated with 2 doses of 600 RAD, reconstituted with a total of 5×106 bone marrow cells from donors, and allowed to reconstitute 6 weeks before analysis (Mean+/−SE *=p<0.05 n=5; results expressed as percentage of live cells). For the whole bone marrow mixed chimera 2.5×10⁶ cells from each donor were reconstituted into the recipient (Mean+/−SE *=p<0.05 n=4; results expressed as percent of cell population). For the MDP transfer approximately 2×10⁴ cells were reconstituted. (Mean+/−SE *=p<0.05 n=6; results expressed as percent of cell population).

FIGS. 4A-4G show: A. Apoptosis detection in Ly6C+ and − monocytes in bone marrow, spleen and blood or myeloid stem cell populations in Nur77 global knockout or BL6 control mice as measured by flow cytometric analysis of Annexin V staining (left). (Mean+/−SE *=p<0.05 n=6). B. (left) Representative scatterplot of Annexin V staining to detect apoptosis and Propidium Iodide staining to detect cell death in bone marrow Ly6C− monocytes from Nur77−/− or BL6 control mice. (right) Quantification of apoptotic and dead cells measured by Annexin V and Propidium Iodide staining. (Mean+/−SE *=p<0.05 n=6) C. Percent cells with active Caspase-3 staining in bone marrow monocytes from Nur77−/− or BL6 mice using a fluorescent conjugated cleaved (active) caspase 3 specific antibody by flow cytometry. (Mean+/−SE *=p<0.05 n=8) D. Representative cleaved (active) caspase 3 and DAPI (nuclear) immunofluorescent staining of Ly6C− bone marrow monocytes isolated by FACS from Nur77−/− or BL6 mice. (scale bar=5 μm) E. Relative activity of p65 (pS529) activity in bone marrow, blood and spleen monocytes from Nur77−/− or BL6 control mice as measured by intracellular flow cytometric analysis using an anti p65 phosphoserine 529 antibody. (Mean+/−SE *=p<0.05 n=4) F. Relative expression of IKBα transcripts in Ly6C− monocytes isolated by FACS from bone marrow of Nur77−/− or BL6 mice measured by qRT-PCR. (Mean+/−SE *=p<0.05 n=6, expressed as a percentage of BL6 transcript). G. Nuclear co-localization of p65 in bone marrow Ly6C− monocyte populations from Nur77−/− and BL6 control mice measured by immunofluorescent staining. Monocytes were sorted by FACS, fixed on slides and then stained with p65 antibody and DAPI staining of the nucleus. The percent of p65 staining co-localized in the nucleus was quantified using Imaris software. (Mean+/−SE *=p<0.05 n=6).

FIGS. 5A-5C show: A. Representative flow cytometry analysis of cell cycle progression in bone marrow Ly6C− monocytes from BL6 control or Nur77−/− mice stained with propidium iodide. Gates show percentage of cells in G1/0, S and G2 phase of the cell cycle from left to right. B. Quantification of (A) expressed as an average percentage of cells in each phase of cell cycle (Mean+/−SE *=p<0.05 n=6). C. Relative expression of Cyclin A2, Cyclin D2, Cdk1 and E2F2 transcripts in Ly6C− monocytes isolated by FACS from bone marrow of Nur77−/− or BL6 mice and measured by qRT-PCR. (Mean+/−SE *=p<0.05 n=6, expressed as a percentage of BL6 transcript).

FIGS. 6A-6E show: A. Expression of CCR2, CX3CR1, and LFA-1 (CD11a) in monocyte populations from Nur77−/− or BL6 control bone marrow analyzed by flow cytometry. CCR2 expression was measured using the MC21 antibody by established methods (Mack et al.), and CX3CR1 expression was measured using the T-20 antibody along with anti-goat PE (Santa Cruz Biotechnology). (Mean+/−SE *=p<0.05 n=5) B. Relative expression of CX3CR1, CEBPβ, JunB and PU.1 transcripts in Ly6C− monocytes isolated by FACS from bone marrow of Nur77−/− or BL6 mice measured by qRT-PCR. (Mean+/−SE *=p<0.05 n=6, expressed as a percentage of BL6 transcript) C. Representative CD11b+ tracking data for C57BL6 BM recipients (upper 2 panels), and Nur77−/− BM recipients (lower 2). Anaesthetised mice were injected i.v. with 10 μg PE conjugated anti-mouse CD11b (M1/70; Becton Dickinson) and cell tracks (left) and displacement vectors of individual cells (red arrows, right) are shown. D. Mean number (±SD) of patrolling CD11b+ cells/field/hrs in CX3CR1gfp/+, RAG2−/−, γc−/− mice, and C57BL6 and Nur77−/− bone marrow chimera. Scale bars represent 60 μm. (Mean+/−SE *=p<0.05, for Nur77−/− BM-chimera represent 7 fields/hrs and 4 animals, for C57BL6 BM-chimera 3 fields/hrs and 2 animals, and CX3CR1gfp/+, RAG2−/−, γc−/−12 fields/hrs, and 3 animals) E. Blood monocyte subsets from C57BL6 or Nur77−/− bone marrow chimera analyzed by flow cytometry. Lineage negative (NK1.1, CD19, CD3) CD115+ cells were analyzed for expression of Ly6C, CD45.2 (donor), and CD45.1 (recipient). Chimerism was 98% for both C57BL6 and Nur77−/− bone marrow chimera. Ly6Clow Lin− CD115+ cells represented 33±1.4% of monocytes in C57BL6 bone marrow chimera, but 4±1.4% in Nur77−/− BM recipients.

FIG. 7 shows human blood monocytes incubated with M-CSF (1200 ng/ml) for 6 days to produce monocyte-derived macrophages (M0). These macrophages were incubated with Gal-3BP (10 μg/ml) for 0-72 hours. Nur77 mRNA was measured by real-time RT-PCR and normalized according to 2-ΔΔCt method using H185 as a housekeeping/normalizing gene. In two independent studies, NUR77 mRNA was increased 3-4 fold by BTBD17B (galectin-3 binding protein or Gal-3BP) with peak expression at 48 hours after addition of Gal-3BP. Nur77 expression was not seen when macrophages were pre-treated with cyclosporineA (CSA), a known NFAT inhibitor.

FIG. 8 shows expression of NR4A1 in response to Gal-3BP (solid line) or LPS (dashed line) at 0, 1, 6 and 24 hours after addition of stimulus.

FIGS. 9A-9G show that Galectin-3 binding protein regulates oxLDL uptake through nuclear receptor NR4A1. A-B. Primary human monocyte-derived macrophages were left untreated or exposed to recombinant human Gal-3BP (10 μg/ml) for 48 hours and stained for Nur77 (red) and nuclei (blue, scale bar=10 μm). C. In parallel studies, NR4A1mRNA expression was measured after six hours by real-time RT-PCR and normalized to t=0 (n=3). D-G. Bone marrow cells were harvested from wild-type and Nr4a1−/− mice, differentiated with GM-CSF (8 days, 30 ng/ml) and exposed to DiI-labeled oxLDL (10 μg/ml) for 4 hours. Intensity histograms for wild-type (dashed lines) and Nr4a1−/− (solid lines) cells and average mean fluorescence intensity (MFI) for all cells (D) or CD11b+CD11c+ cells (F). Negative control (no oxLDL) shown in shaded histogram. Average MFI for all (E) and CD11b+CD11c+ cells (G). Triplicate samples of pooled mice, representative of two independent studies. *P<0.05, **P<0.01.

FIG. 10 shows increased atherosclerosis in mice receiving Nur77−/− bone marrow. BMT of female B6 or Nur77−/− marrow was performed into female ldlr−/− recipients. Mice were fed a Western diet for 18 weeks.

### DETAILED DESCRIPTION

The invention is based, at least in part, on the identification of Nur77 (also known as NR4A1 or NGFI-B) as a modulator of immune, inflammatory responses and inflammation. In particular, as disclosed herein Nur77, for example, is required for differentiation and/or survival of Ly6C− monocytes from bone marrow progenitor cells, and/or patrolling function of Ly6C− monocytes in blood vessels. The absence of Nur77 also results in increased atherosclerosis indicating that Nur77 inhibits development of atherosclerosis, either due to decreasing the pro-inflammatory phenotype of monocytes, or maintaining, promoting, or increasing Nur77 dependent monocytes that inhibit atherosclerosis. Nur77 therefore has a significant role in the regulation of Ly6C− monocyte differentiation and survival, macrophages, and their effector functions in vivo.

Accordingly, the invention provides, inter alia, Nur77 compositions, and methods and uses of Nur77 polypeptides, Nur77 agonists and Nur77 regulated monocytes (e.g., CD14⁺ CD16⁺ or CD14^{dim} CD16⁺ monocytes or macrophages). Methods and uses include, for example, treatment of undesirable and aberrant immune responses, immune disorders, inflammatory responses, inflammation and autoimmune responses, disorders and diseases, among others, in a subject. Methods and uses also include, for example, treatment of autoimmune responses, disorders and diseases in a subject. Methods and uses further include, for example, treatment of an adverse cardiovascular event or cardiovascular disease in a subject.

Compositions, methods and uses of the invention include Nur77 polypeptides and subsequences and fragments of Nur77 polypeptides. Two exemplary full length human Nur77 polypeptide sequences, as disclosed herein, have an amino acid length of about 611 and 598 amino acid residues (SEQ ID NO:1 and 2) as follows:

Human NR4A1 protein sequence (Homo sapiens nuclear receptor subfamily 4, group A, member 1 (NR4A1):

>NP_001189162 length = 611
MWLAKACWSIQSEMPCIQAQYGTPAPSPGPRDHLASDPLTPEFIKPTMDL
ASPEAAPAAPTALPSFSTFMDGYTGEFDTFLYQLPGTVQPCSSASSSASS
TSSSSATSPASASFKFEDFQVYGCYPGPLSGPVDEALSSSGSDYYGSPCS
APSPSTPSFQPPQLSPWDGSFGHFSPSQTYEGLRAWTEQLPKASGPPQPP
AFFSFSPPTGPSPSLAQSPLKLFPSQATHQLGEGESYSMPTAFPGLAPTS
PHLEGSGILDTPVTSTKARSGAPGGSEGRCAVCGDNASCQHYGVRTCEGC
KGFFKRTVQKNAKYICLANKDCPVDKRRRNRCQFCRFQKCLAVGMVKEVV
RTDSLKGRRGRLPSKPKQPPDASPANLLTSLVRAHLDSGPSTAKLDYSKF
QELVLPHFGKEDAGDVQQFYDLLSGSLEVIRKWAEKIPGFAELSPADQDL
LLESAFLELFILRLAYRSKPGEGKLIFCSGLVLHRLQCARGFGDWIDSIL
AFSRSLHSLLVDVPAFACLSALVLITDRHGLQEPRRVEELQNRIASCLKE
HVAAVAGEPQPASCLSRLLGKLPELRTLCTQGLQRIFYLKLEDLVPPPPI
IDKIFMDTLPF

Homo sapiens nuclear receptor subfamily 4, group A, member 1 (NR4A1), with representative amino acid polymorphisms illustrated by bold/underlining:

>NP_002126 length = 598 >NP_775180 length = 598

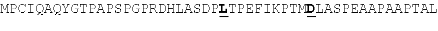

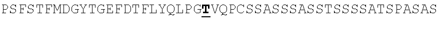

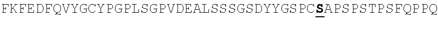

LSPWDGSFGHFSPSQTYEGLRAWTEQLPKASGPPQPPAFFSFSPPTGPSP
SLAQSPLKLFPSQATHQLGEGESYSMPTAFPGLAPTSPHLEGSGILDTPV

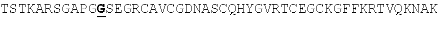

YICLANKDCPVDKRRRNRCQFCRFQKCLAVGMVKEVVRTDSLKGRRGRLP

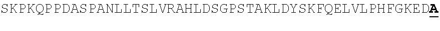

GDVQQFYDLLSGSLEVIRKWAEKIPGFAELSPADQDLLLESAFLELFILR
LAYRSKPGEGKLIFCSGLVLHRLQCARGFGDWIDSILAFSRSLHSLLVDV
PAFACLSALVLITDRHGLQEPRRVEELQNRIASCLKEHVAAVAGEPQPAS
CLSRLLGKLPELRTLCTQGLQRIFYLKLEDLVPPPPIIDKIFMDTLPF

More specifically, representative polymorphisms include one or more of the following seven amino acid substitutions of Nur77:

1) SNP rs1882118—(L) Leu to (V) Val at aa position 26
2) SNP rs113544195—(D) Asp to (G) Gly at aa position 36
3) SNP rs75476334—(T) Thr to (I) Ile at aa position 74
4) SNP rs61734310—(S) Ser to (L) Leu at aa position 137
5) SNP rs1042315—(G) Gly to (R) Arg at aa position 262
6) SNP rs1042316—(G) Gly to (A) Ala at aa position 262
7) SNP rs61751044—(A) Ala to (D) Asp at aa position 400
Such full length mammalian (human) Nur77 polypeptide sequences, fragments/subsequences, polymorphisms and modified forms and variants as set forth herein, are also included as invention compositions, methods and uses.

As used herein, a “polypeptide” refers to two, or more, amino acids linked by an amide or equivalent bond. A polypeptide can also be referred to herein, inter alia, as a protein, peptide, or an amino acid sequence. Polypeptides can form ultra or intermolecular disulfide bonds. Polypeptides can also form higher order structures, such as multimers or oligomers, with the same or different polypeptide, or other molecules.

A Nur77 polypeptide refers to full length polypeptide sequence, as well as subsequences, fragments or portions, polymorphisms, variants and modified forms of Nur77 polypeptide, unless the context indicates otherwise. Such Nur77 subsequences, fragments, polymorphisms, variants and modified forms have at least a part of, a function or activity of an unmodified or reference Nur77 protein. In particular embodiments, a polymorphism, variant or modified form retains, at least a part of, a function or activity of a reference Nur77 protein. A “functional polypeptide” or “active polypeptide” refers to a polymorphic, variant or modified polypeptide or subsequence thereof that possesses at least one partial function or activity (e.g., biological activity) characteristic of a native wild type or full length counterpart polypeptide, for example, Nur77 polypeptides of SEQ ID NOs:1 or 2, as disclosed herein, which function or activity can be identified through an assay. The invention therefore includes polymorphisms, variants and modified forms of Nur77 polypeptide sequences, and such subsequences, polymorphisms, variants or modified forms typically retain, at least a part of, one or more functions or activities of a reference or an unmodified Nur77 polypeptide sequence.

As disclosed herein, particular non-limiting examples of a function or activity of Nur77 polypeptide is to promote, stimulate, enhance, or increase production, development, survival, proliferation, differentiation, survival, patrolling, homing or activity of Ly6C− type monocytes, and one or more monocyte effector functions. Such Ly6C− type monocytes include human moncoytes, which are characterized as CD14⁺ CD16⁺ monocytes or CD14^{dim}CD16⁺ (CD115⁺CD11b⁺GR1⁻ (Ly6C−)) monocytes. Accordingly, Nur77 polypeptides, subsequences and variants as well as Nur77 agonists can be used to promote, stimulate, enhance, or increase production, development, survival, proliferation, differentiation, survival, patrolling, homing or activity of Ly6C− type monocytes and their human equivalents (e.g., CD14⁺ CD16⁺ monocytes or CD14^{dim}CD16⁺ (CD115⁺CD11b⁺GR1) monocytes) and/or one or more monocyte effector functions in vitro and/or in vivo.

Additional particular non-limiting examples of a function or activity of Nur77 polypeptide is to decrease, reduce, inhibit, suppress, limit or control an undesirable or aberrant immune response, immune disorder, inflammatory response, or inflammation, or an autoimmune response, disorder or disease. Further non-limiting examples of a function or activity of Nur77 polypeptide is to decrease, reduce, inhibit, suppress, limit or control an adverse cardiovascular event or cardiovascular disease in a subject.

Accordingly, Nur77 polypeptide sequences, fragments/subsequences, polymorphisms, variants and modified forms can have one or more of the foregoing functions or activities, or other functions or activities attributed to wild type native Nur77 polypeptide. Compositions, methods and uses of the invention therefore include Nur77 polypeptide sequences, fragments/subsequences, polymorphisms, variants and modified forms having one or more functions or activities of wild type native Nur77 polypeptide.

Compositions, methods and uses of the invention also include Nur77 agonists, as such agonists can promote, stimulate, enhance, or increase activity, function or expression of Nur77 polypeptide. Accordingly, Nur77 agonists can function as Nur77 polypeptide sequences, fragments/subsequences, polymorphisms, variants and modified forms, and as such be used in accordance with the invention compositions, methods and uses.

Non-limiting exemplary Nur77 agonists include 9-cis-retinoic acid; 1-di(3-indolyl)-1-(4-X-phenyl)methanes; etoposide; 5,8-diacetoxyl-6-(1′-acetoxy-4′-methyl-3′-pentenyl)-1,4-naphthaquinones; 12-O-tetradecanoylphorbol-13-acetate; diindolylmethane analogs (C-DIMs), such as 1,1-bis(3′-indolyl)-1-(p-methoxyphenyl)methane (DIM-C-pPhOCH3); 6-mercaptopurine; and panobinostat ((2E)-N-hydroxy-3-[4-({[2-(2-methyl-1H-indol-3-yl)ethyl]amino}methyl)phenyl]acrylamide). Additional Nur77 agonists include octaketide Cytosporone B (Csn-B) and derivatives thereof, such as n-amyl 2-[3,5-dihydroxy-2-(1-nonanoyl)phenyl]acetate or a compound having a structure set forth in Table 1.

TABLE 1
Structures of exemplary Csn-B derived Nur77 agonists (compounds 1-15)

| | |
|---|---|
| 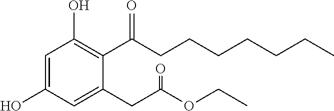 [SMILES: CCCCCCCC(=O)c1c(O)cc(O)cc1CC(=O)OCC] | 1 |
| 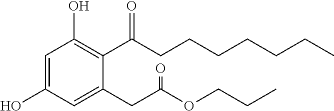 [SMILES: CCCCCCCC(=O)c1c(O)cc(O)cc1CC(=O)OCCC] | 2 |
| 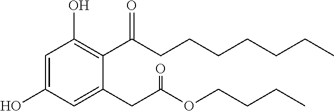 [SMILES: CCCCCCCC(=O)c1c(O)cc(O)cc1CC(=O)OCCCC] | 3 |
| 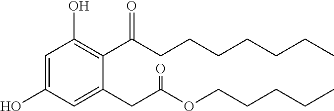 [SMILES: CCCCCCCC(=O)c1c(O)cc(O)cc1CC(=O)OCCCCC] | 4 |
| 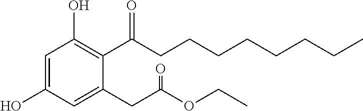 [SMILES: CCCCCCCCC(=O)c1c(O)cc(O)cc1CC(=O)OCC] | 5 |
| 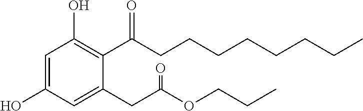 [SMILES: CCCCCCCCC(=O)c1c(O)cc(O)cc1CC(=O)OCCC] | 6 |
| 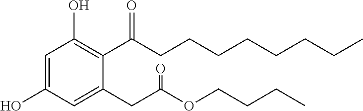 [SMILES: CCCCCCCCC(=O)c1c(O)cc(O)cc1CC(=O)OCCCC] | 7 |
| 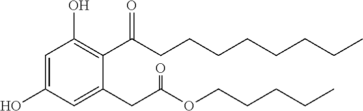 [SMILES: CCCCCCCCC(=O)c1c(O)cc(O)cc1CC(=O)OCCCCC] | 8 |
| 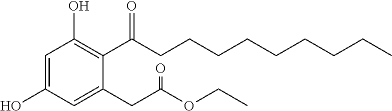 [SMILES: CCCCCCCCCC(=O)c1c(O)cc(O)cc1CC(=O)OCC] | 9 |
| 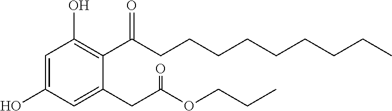 [SMILES: CCCCCCCCCC(=O)c1c(O)cc(O)cc1CC(=O)OCCC] | 10 |
| 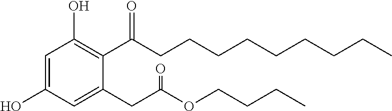 [SMILES: CCCCCCCCCC(=O)c1c(O)cc(O)cc1CC(=O)OCCCC] | 11 |
| 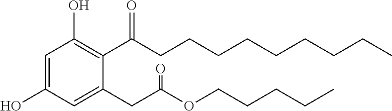 [SMILES: CCCCCCCCCC(=O)c1c(O)cc(O)cc1CC(=O)OCCCCC] | 12 |
| 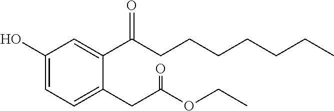 [SMILES: CCCCCCCC(=O)c1cc(O)ccc1CC(=O)OCC] | 13 |
| 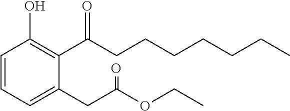 [SMILES: CCCCCCCC(=O)c1c(O)cccc1CC(=O)OCC] | 14 |
| 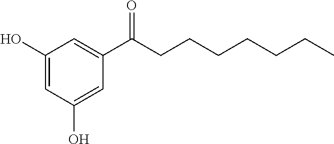 [SMILES: CCCCCCCC(=O)c1cc(O)cc(O)c1] | 15 |

As used herein, the term “modify” and grammatical variations thereof, means that the composition deviates from a reference composition. Modifications include, for example, substitutions, additions, insertions, deletions and other variations to the amino acid sequences set forth herein, which can be referred to as “variants.” The invention compositions, methods and uses include such variant Nur77 polypeptides, including Nur77 polymorphisms.

Exemplary sequence substitutions, additions, and insertions include a full length or a portion of a sequence with one or more amino acids substituted, added or inserted. For example, a variant Nur77 polypeptide has one or more functions or activities of wild type Nur77, including without limitation one or more functions or activities of Nur77 as set forth herein. Such exemplary variant Nur77 polypeptide sequences include Nur77 polymorphisms. Exemplary Nur77 polymorphisms include one or more of the following amino acid substitutions, without limitation: (L) Leu to (V) Val at aa position 26; (D) Asp to (G) Gly at aa position 36; (T) Thr to (I) Ile at aa position 74; (S) Ser to (L) Leu at aa position 137; (G) Gly to (R) Arg at aa position 262; (G) Gly to (A) Ala at aa position 262; and (A) Ala to (D) Asp at aa position 400.

Additional non-limiting examples of variant polypeptides include, for example, non-conservative and conservative substitutions of Nur77 polypeptide sequences. In particular embodiments, a variant Nur77 protein has one or a few (e.g., 1-5%, 5-10%, 10-20% or 20-30% of the residues of total protein length, or 2-10, 10-20, 20-30, 30-40, 40-50, 50-60, 60-75, 75-100 residues, substituted) conservative or non-conservative substitutions.

As used herein, the term “conservative substitution” denotes the replacement of an amino acid residue by another, chemically or biologically similar residue. Biologically similar means that the substitution does not destroy a biological activity or function. Structurally similar means that the amino acids have side chains with similar length, such as alanine, glycine and serine, or a similar size. Chemical similarity means that the residues have the same charge or are both hydrophilic or hydrophobic. Particular examples of conservative substitutions include the substitution of a hydrophobic residue such as isoleucine, valine, leucine or methionine for another, the substitution of a polar residue for another, such as the substitution of arginine for lysine, glutamic for aspartic acids, or glutamine for asparagine, and the like. The term “conservative substitution” also includes the use of a substituted amino acid in place of an unsubstituted parent amino acid.

Modified proteins also include one or more D-amino acids substituted for L-amino acids (and mixtures thereof), structural and functional analogues, for example, peptidomimetics having synthetic or non-natural amino acids or amino acid analogues and derivatized forms. Modifications include cyclic structures such as an end-to-end amide bond between the amino and carboxy-terminus of the molecule or ultra- or inter-molecular disulfide bond.

Modified forms further include “chemical derivatives,” in which one or more amino acids have a side chain chemically altered or derivatized. Such derivatized polypeptides include, for example, amino acids in which free amino groups form amine hydrochlorides, p-toluene sulfonyl groups, carobenzoxy groups; the free carboxy groups form salts, methyl and ethyl esters; free hydroxl groups that form O-acyl or O-alkyl derivatives as well as naturally occurring amino acid derivatives, for example, 4-hydroxyproline, for proline, 5-hydroxylysine for lysine, homoserine for serine, ornithine for lysine etc. Also included are amino acid derivatives that can alter covalent bonding, for example, the disulfide linkage that forms between two cysteine residues that produces a cyclized polypeptide. Further modified forms include sugars, or glycosylated proteins.

As set forth herein, variant and modified forms include additions and insertions. For example, an addition can be one or more amino acid residues, or a covalent or non-covalent attachment of any type of molecule to a protein (e.g., Nur77) or other composition (e.g., Nur77 agonist). Particular examples of additions and insertions are entities that confer a complementary or a distinct function or activity.

Exemplary additions and insertions include fusion or chimeric polypeptide sequence constructs, which is a sequence having one or more molecules not normally present in a reference native (wild type) sequence (e.g., Nur77) covalently attached to the sequence. The terms “fusion” or “chimeric” and grammatical variations thereof, when used in reference to a molecule, such as a Nur77 or a Nur77 agonist, means that a portions or part of the molecule contains a different entity distinct from the molecule (e.g., Nur77 or agonist) as they do not typically exist together in nature. That is, for example, one portion of the fusion or chimera, such as Nur77, includes or consists of a portion that does not exist together in nature, and is structurally distinct. A particular example is an amino acid sequence of another protein (e.g., immunoglobulin such as an Fc domain, or antibody) attached to Nur77 or Nur77 agonist to produce a fusion, or a chimeric polypeptide, to impart a distinct function (e.g., multifunctional, increased solubility, in vivo half life, etc.).

Additions and insertions also include a label or a tag, which can be used to provide an agent that is detectable or that is useful for isolating the tagged entitiy (e.g., Nur77, Nur77 agonist). A detectable label can be attached, for example, to (e.g., linked conjugated) Nur77, or Nur77 agonist, or be within or be one or more atoms that comprise the molecule.

Non-limiting exemplary detectable labels include contrast agents (e.g., gadolinium; manganese; barium sulfate; an iodinated or noniodinated agent; an ionic agent or nonionic agent); magnetic and paramagnetic agents (e.g., iron-oxide chelate); nanoparticles; an enzyme (horseradish peroxidase, alkaline phosphatase, β-galactosidase, or acetylcholinesterase); a prosthetic group (e.g., streptavidin/biotin and avidin/biotin); a fluorescent material (e.g., umbelliferone, fluorescein, fluorescein isothiocyanate, rhodamine, dichlorotriazinylamine fluorescein, dansyl chloride or phycoerythrin); a luminescent material (e.g., luminol); or a bioluminescent material (e.g., luciferase, luciferin, aequorin).

Additional non-limiting examples of detectable labels and/or tags include enzymes (horseradish peroxidase, urease, catalase, alkaline phosphatase, beta-galactosidase, chloramphenicol transferase); enzyme substrates; ligands (e.g., biotin); receptors (avidin); GST-, T7-, His-, myc-, HA- and FLAG-tags; electron-dense reagents; energy transfer molecules; paramagnetic labels; fluorophores (fluorescein, fluorscamine, rhodamine, phycoerthrin, phycocyanin, allophycocyanin); chromophores; chemi-luminescent (imidazole, luciferase, acridinium, oxalate); and bio-luminescent agents.

As set forth herein, a tag or detectable label can be linked or conjugated (e.g., covalently) to the molecule (e.g., Nur77, Nur77 agonist). In various embodiments a detectable label, such as a radionuclide or metal or metal oxide can be bound or conjugated to the agent, either directly or indirectly. A linker or an intermediary functional group can be used to link the molecule to a detectable label or tag. Linkers include amino acid or peptidomimetic sequences inserted between the molecule and a label or tag so that the two entities maintain, at least in part, a distinct function or activity. Linkers may have one or more properties that include a flexible conformation, an inability to form an ordered secondary structure or a hydrophobic or charged character which could promote or interact with either domain Amino acids typically found in flexible protein regions include Gly, Asn and Ser. The length of the linker sequence may vary without significantly affecting a function or activity.

Linkers further include chemical moieties, conjugating agents, and intermediary functional groups. Examples include moieties that react with free or semi-free amines, oxygen, sulfur, hydroxy or carboxy groups. Such functional groups therefore include mono and bifunctional crosslinkers, such as sulfo-succinimidyl derivatives (sulfo-SMCC, sulfo-SMPB), in particular, disuccinimidyl suberate (DSS), BS3 (Sulfo-DSS), disuccinimidyl glutarate (DSG) and disuccinimidyl tartrate (DST). Non-limiting examples include diethylenetriaminepentaacetic acid (DTPA) and ethylene diaminetetracetic acid.

Additional non-limiting examples of amino acid modifications and variants include protein subsequences and fragments. A subsequence, fragment or portion of Nur77 polypeptide means one or more amino acids fewer than the full length reference sequence, which is typically a native full length Nur77 polypeptide sequence. Deletion of one or more amino acids can result in a modification of the structure of the resultant polypeptide without significantly altering a biological function or activity. Exemplary subsequences and fragments therefore include a Nur77 polypeptide fragment or a portion thereof that promotes, stimulates, enhances, or increases production, development, survival, proliferation, differentiation, survival, patrolling, homing or activity of Ly6C− type monocytes (or human equivalents, e.g., CD14⁺ CD16⁺ monocytes or CD14^{dim}CD16⁺ (CD115⁺CD11b⁺GR1⁻ monocytes), and/or one or more monocyte effector functions; a Nur77 polypeptide fragment or a portion thereof that decreases, reduces, inhibits, suppresses, limits or controls an undesirable or aberrant immune response, immune disorder, inflammatory response, inflammation, an autoimmune response, disorder or disease; and a Nur77 polypeptide fragment or a portion thereof that decreases, reduces, inhibits, suppresses, limits or controls an adverse cardiovascular event or cardiovascular disease.

Non-limiting subsequences of full length Nur77 include amino acids having a length of about 10-20, 20-25, 25-50, 50-100, 100-150, 150-200, 200-250, 250-300, 300-350, 350-400, 400-500, 500-600 or more amino acids in length, but less than a full length Nur77 polypeptide sequence, e.g., a native (naturally occurring) sequence. As disclosed herein, Nur77 subsequences, fragments and portions can retain all or a part of a function or activity of full length Nur77 polypeptide (e.g., decreases, reduces, inhibits, suppresses, limits or controls an undesirable or aberrant immune response, immune disorder, inflammatory response, inflammation, an autoimmune response, disorder or disease, or an adverse cardiovascular event or cardiovascular disease, or promotes, stimulates, enhances, or increases production, development, survival, proliferation, differentiation, survival, patrolling, homing or activity of Ly6C− type monocytes (or human equivalents, e.g., CD14⁺ CD16⁺ monocytes or CD14^{dim}CD16⁺ (CD115⁺CD11b⁺GR1⁻ monocytes), and/or one or more monocyte effector functions, etc.

Modified and variant Nur77 polypeptide sequences and subsequences of the invention may have an activity or function greater or less than 2-5, 5-10, 10-100, 100-1000 or 1000-10.000-fold activity or function than a comparison Nur77 polypeptide sequence or subsequence (e.g, SEQ ID NOs:1 or 2). For example, a modified Nur77 polypeptide sequences or subsequence could have an activity or function greater or less than 2-5, 5-10, 10-100, 100-1000 or 1000-10.000-fold activity or function of a reference Nur77 to decrease, reduce, inhibit, suppress, limit or control an undesirable or aberrant immune response, immune disorder, inflammatory response, inflammation, an autoimmune response, disorder or disease, an adverse cardiovascular event or cardiovascular disease, or to promote, stimulate, enhance, or increase production, development, survival, proliferation, differentiation, survival, patrolling, homing or activity of Ly6C− type monocytes (or human equivalents, e.g., CD14⁺ CD16⁺ monocytes or CD14^{dim}CD16⁺ (CD115⁺CD11b⁺GR1⁻ monocytes), and/or one or more monocyte effector functions, etc.

Nur77 polypeptide variants also include sequences having less than 100% identity to a reference Nur77 polypeptide sequence. Such Nur77 polypeptide sequences can have at least about 60%, 65%, 70%, 75%, 80%, 85%, 90%, 95%, 96%, 97%, 98%, 99% or more identity (homology) to a reference Nur77 polypeptide sequence (e.g., a mammalian Nur77 polypeptide sequence, such as human Nur77 polypeptide sequence set forth as SEQ ID NOs:1 or 2). Such Nur77 polypeptide sequences with at least about 60%, 65%, 70%, 75%, 80%, 85%, 90%, 95%, 96%, 97%, 98%, 99% or more identity (homology) to a reference Nur77 sequence can have sufficient identity to retain all or a part of a function or activity of a reference Nur77 polypeptide (e.g., decreases, reduces, inhibits, suppresses, limits or controls an undesirable or aberrant immune response, immune disorder, inflammatory response, inflammation, an autoimmune response, disorder or disease, an adverse cardiovascular event or cardiovascular disease, or promotes, stimulates, enhances, or increases production, development, survival, proliferation, differentiation, survival, patrolling, homing or activity of Ly6C− type monocytes (or human equivalents, e.g., CD14⁺ CD16⁺ monocytes or CD14^{dim}CD16⁺ (CD115⁺CD11b⁺GR1⁻ monocytes), and/or one or more monocyte effector functions.

The term “identity” and grammatical variations thereof, mean that two or more referenced entities are the same. Thus, where two polypeptide sequences (e.g., Nur77 polypeptide sequences) are identical, they have the same amino acid sequence, at least within the referenced region or portion. The identity can be over a defiled area (region or domain) of the sequence. An “area of identity” refers to a portion of two or more referenced entities that are the same. Thus, where two protein sequences are identical over one or more sequence regions they share identity within that region.

The percent identity can extend over the entire sequence length of the polypeptide (e.g., a Nur77 polypeptide sequence). In particular aspects, the length of the sequence sharing the percent identity is 5 or more contiguous amino acids, e.g., 5, 6, 7, 8, 9, 10, 11, 12, 13, 14, 15, 16, 17, 18, 19, 20, 21, 22, 23, 24, 25, etc. contiguous amino acids. In additional particular aspects, the length of the sequence sharing the percent identity is 25 or more contiguous amino acids, e.g., 26, 27, 28, 29, 30, 31, 32, 33, 34, 35, etc. contiguous amino acids. In further particular aspects, the length of the sequence sharing the percent identity is 35 or more contiguous amino acids, e.g., 35, 36, 37, 38, 39, 40, 41, 42, 43, 44, 45, 45, 47, 48, 49, 50, etc., contiguous amino acids. In yet additional particular aspects, the length of the sequence sharing the percent identity is 50 or more contiguous amino acids, e.g., 50-55, 55-60, 60-65, 65-70, 70-75, 75-80, 80-85, 85-90, 90-95, 95-100, 100-110, etc. contiguous amino acids.

The extent of identity (homology) between two sequences can be ascertained using a computer program and mathematical algorithm known in the art. Such algorithms that calculate percent sequence identity (homology) generally account for sequence gaps and mismatches over the comparison region or area. For example, a BLAST (e.g., BLAST 2.0) search algorithm (see, e.g., Altschul et al., J. Mol. Biol. 215:403 (1990), publicly available through NCBI) has exemplary search parameters as follows: Mismatch −2; gap open 5; gap extension 2. For polypeptide sequence comparisons, a BLASTP algorithm is typically used in combination with a scoring matrix, such as PAM100, PAM 250, BLOSUM 62 or BLOSUM 50. FASTA (e.g., FASTA2 and FASTA3) and SSEARCH sequence comparison programs are also used to quantitate extent of identity (Pearson et al., Proc. Natl. Acad. Sci. USA 85:2444 (1988); Pearson, Methods Mol Biol. 132:185 (2000); and Smith et al., J. Mol. Biol. 147:195 (1981)). Programs for quantitating protein structural similarity using Delaunay-based topological mapping have also been developed (Bostick et al., Biochem Biophys Res Commun. 304:320 (2003)).

Modifications and variants can be produced using methods known in the art (e.g., PCR based site-directed, deletion and insertion mutagenesis, chemical modification and mutagenesis, cross-linking, etc.), or may be spontaneous or naturally occurring (e.g. random mutagenesis). For example, naturally occurring Nur77 polypeptide sequence allelic variants can occur by alternative RNA splicing, polymorphisms, or spontaneous mutations of a nucleic acid encoding Nur77 polypeptide. Additions and deletions of one or more amino acids can also be produced using molecular genetic techniques known to the skill artisan.

Invention compositions, methods and uses include isolated and purified Nur77 polypeptides, variants and modified forms, such as subsequences and fragments and polymorphisms of Nur77 polypeptides. The term “isolated,” when used as a modifier of a composition, means that the composition is made by the hand of man or are separated, completely or at least in part, from their naturally occurring in vivo environment. The term “isolated” does not exclude alternative physical forms of the composition, such as fusions/chimeras, multimers/oligomers, modifications (e.g., phosphorylation, glycosylation, lipidation) or derivatized forms, or forms expressed in host cells produced by the hand of man.

An “isolated” composition (e.g., a Nur77 polypeptide sequence or Nur77 agonist) can also be “substantially pure” or “purified” when free of most or all of the materials with which it typically associates with in nature. Generally, isolated compositions are substantially free of one or more materials with which they normally associate with in nature, for example, one or more protein, nucleic acid, lipid, carbohydrate, cell membrane. Thus, an isolated sequence that also is substantially pure or purified does not include polypeptides or polynucleotides present among millions of other sequences, such as antibodies of an antibody library or nucleic acids in a genomic or cDNA library, for example. Typically, purity can be at least about 50%, 60% or more by mass. The purity can also be about 70% or 80% or more, and can be greater, for example, 90% or more. Purity can be determined by any appropriate method, including, for example, UV spectroscopy, chromatography (e.g., HPLC, gas phase), gel electrophoresis and sequence analysis (nucleic acid and peptide), and is typically relative to the amount of impurities, which typically does not include inert substances, such as water.

A “substantially pure” or “purified” composition can be combined with one or more other molecules. Thus, “substantially pure” or “purified” does not exclude combinations of compositions, such as combinations of Nur77 polypeptide sequences, subsequences, variants/modified forms (e.g., polymorphisms), or Nur77 agonists. For example, a composition can include a combination of Nur77 polypeptide, and/or a Nur77 agonist, and/or an anti-inflammatory drug or agent.

As used herein, the term “recombinant,” when used as a modifier of polypeptides, means that the compositions have been manipulated (i.e., engineered) in a fashion that generally does not occur in nature (e.g., in vitro). A particular example of a recombinant polypeptide would be where a Nur77 polypeptide is expressed by a cell transfected with a polynucleotide encoding the Nur77 polypeptide. Another example of a recombinant polypeptide is a hybrid or fusion sequence, such as a chimeric or fusion Nur77 polypeptide sequence comprising and a second sequence, such as a heterologous functional domain (e.g., an immunoglobulin sequence, such as an Fc domain).

Invention compositions, methods and uses that include Nur77 polypeptide or Nur77 agonist or CD14⁺ CD16⁺ or CD14^{dim} CD16⁺ monocytes or macrophages can include any amount or dose of Nur77 polypeptide, subsequence, modified form or variant (e.g., polymorphism) or Nur77 agonist or CD14⁺ CD16⁺ or CD14^{dim} CD16⁺ monocytes or macrophages. In particular embodiments, such Nur77 polyeptide or Nur77 agonist is in a concentration range of about 1 μg/ml to 1 mg/ml, or in a range of about 10 μg/ml to 100 mg/ml, or in a range of about 100 μg/ml to 1,000 mg/ml, or at a concentration of about 0.1 mg/ml, 1 mg/ml or 10 mg/ml. In further particular embodiments, Nur77 is in an amount of 10-1,000 milligrams, or an amount of 10-100 milligrams.

Nur77 polypeptides, subsequences, variants and modified forms (e.g., modified or unmodified full length native mammalian, such as human Nur77 polypeptide sequences), Nur77 agonists and CD14⁺ CD16⁺ or CD14^{dim} CD16⁺ monocytes or macrophages as disclosed herein, including compositions including such Nur77 polyeptides, Nur77 agonists, and CD14⁺ CD16⁺ or CD14^{dim} CD16⁺ monocytes or macrophages are useful in various treatment methods and uses. Such Nur77 and Nur77 agonists and CD14⁺ CD16⁺ or CD14^{dim} CD16⁺ monocytes or macrophages and compositions thereof are applicable to uses and treatment methods for numerous disorders and diseases, both chronic and acute.

Responses, disorders and diseases treatable in accordance with the invention include, but are not limited to, treatment of acute and chronic undesirable or aberrant immune response, immune disorder, inflammatory response, or inflammation. Responses, disorders and diseases treatable in accordance with the invention also include, but are not limited to treatment of an acute and chronic autoimmune response, disorder or disease. Responses, disorders and diseases treatable in accordance with the invention further include, but are not limited to, treatment of an adverse cardiovascular event or cardiovascular disease. Such responses, disorders and diseases may be antibody or cell mediated, or a combination of antibody and cell mediated.

In accordance with the invention, there are provided compositions, methods and uses for treating acute and chronic responses, disorders and diseases in which a subject would benefit from (e.g., is in need of treatment with) a Nur77 polypeptide sequence or Nur77 agonist or Ly6C− type monocytes (or human equivalents, e.g., CD14⁺ CD16⁺ monocytes or CD14^{dim}CD16⁺ (CD115⁺CD11b⁺GR1⁻ monocytes), and/or one or more effector functions of the monocytes. In various non-limiting embodiments of such methods and uses, a Nur77 polypeptide (or fragment or variant thereof) or Nur77 agonist or Ly6C− type monocytes (or human equivalents, e.g., CD14⁺ CD16⁺ monocytes or CD14^{dim}CD16⁺ (CD115⁺CD11b⁺GR1⁻ monocytes) or macrophages is administered to a subject in an amount intended to achieve a desired effect.

In one embodiment, a method includes administering a Nur77 polypeptide sequence or agonist or CD14⁺ CD16⁺ and/or CD14^{dim} CD16⁺ monocytes or macrophages to a subject, having or at risk of acute or chronic immune response, immune disorder, inflammatory response, or inflammation, an autoimmune response, disorder or disease, in an amount sufficient to treat the acute or chronic undesirable or aberrant immune response, immune disorder, inflammatory response, or inflammation, an autoimmune response, disorder or disease. In another embodiment, a method includes administering a Nur77 polypeptide sequence or agonist or CD14⁺ CD16⁺ and/or CD14^{dim} CD16⁺ monocytes or macrophages to a subject, having or at risk of acute or chronic adverse cardiovascular event or cardiovascular disease in an amount sufficient to treat the adverse cardiovascular event or cardiovascular disease.

In particular aspects, a method decreases, reduces, inhibits, suppresses, limits or controls an undesirable or aberrant immune response, immune disorder, inflammatory response, or inflammation in a subject. In additional particular aspects, a method decreases, reduces, inhibits, suppresses, limits or controls an autoimmune response, disorder or disease in a subject. In further particular aspects, a method decreases, reduces, inhibits, suppresses, limits or controls an adverse cardiovascular event or cardiovascular disease. In yet additional aspects, a method decreases, reduces, inhibits, suppresses, limits or controls an adverse symptom of the undesirable or aberrant immune response, immune disorder, inflammatory response, or inflammation, an adverse symptom of the autoimmune response, disorder or disease or an adverse symptom of the cardiovascular event or cardiovascular disease.

As used herein, an “undesirable or aberrant” immune response, inflammatory response, or inflammation, and grammatical variations thereof, can be a normal or abnormal response, function or activity. Thus, normal immune responses, inflammatory responses, and inflammation that are not considered abnormal or aberrant so long as they are undesirable are included within the meaning of these terms. An abnormal or aberrant immune response, inflammatory response, or inflammation deviates from normal. An abnormal or aberrant immune response, inflammatory response, or inflammation or disorder can be humoral or cellular in nature, or both, either chronic or acute.

Undesirable or aberrant immune responses, inflammatory responses, or inflammation are characterized by many different physiological adverse symptoms or complications, which can be a result be humoral, cell-mediated or a combination thereof. For example, an undesirable or aberrant immune response, inflammatory response, inflammation or autoimmune response, disorder or disease can result in destruction of cells, tissue or organ. Accordingly, responses, disorders and diseases that can be treated in accordance with the invention include, but are not limited to, those that cause cell or tissue/organ damage in a subject.

At the whole body, regional or local level, an immune response, inflammatory response, or inflammation or an adverse cardiovascular event or disease can be characterized by swelling, pain, rash, headache, fever, nausea, diarrhea, bloat, lethargy, skeletal joint stiffness or lack of mobility, pain, rash, redness or other discoloration, stroke, paralysis, loss of vision or other sensory impairment. At the cellular level, an immune response, inflammatory response, or inflammation can be characterized by one or more of cell infiltration of the region, production of antibodies (e.g., auto-antibodies), production of cytokines, lymphokines, chemokines, interferons and interleukins, cell growth and maturation factors (e.g., proliferation and differentiation factors), cell accumulation or migration and cell, tissue or organ damage. Thus, methods and uses of the invention include treatment of and/or an ameliorative effect upon any such physiological symptoms or cellular responses characteristic of immune responses, inflammatory response, inflammation or an autoimmune response, disorder or disease.

Autoimmune responses, disorders and diseases are generally characterized as an undesirable or aberrant response, activity or function of the immune system characterized by increased or undesirable humoral or cell-mediated immune responsiveness or memory, or decreased or insufficient tolerance to self-antigens. Autoimmune responses, disorders and diseases that may be treated in accordance with the invention include but are not limited to responses, disorders and diseases that cause cell or tissue/organ damage in the subject.

Examples of adverse symptoms of an undesirable or aberrant immune response, immune disorder, inflammatory response, or inflammation, or an adverse symptom of an autoimmune response, disorder or disease, or an adverse cardiovascular event or disease include swelling, pain, rash, headache, fever, nausea, diarrhea, bloat, lethargy, skeletal joint stiffness, stroke, pain, paralysis, vision or other sensory impairment, or tissue or cell damage. Additional examples of adverse symptoms of an autoimmune response, disorder or disease include increases in pro-inflammatory cytokines or chemokines. Examples of adverse symptoms occur in particular tissues, or organs, or regions or areas of the body, such as in an epidermal or mucosal tissue, gut, bowel, pancreas, thymus, liver, kidney, muscle, central or peripheral nerves, spleen, skin, or a skeletal joint (e.g., knee, ankle, hip, shoulder, wrist, finger, toe, or elbow), blood vessel (vasculature), or a cardio-pulmonary tissue or organ.

Specific non-limiting examples of aberrant or undesirable immune disorders, inflammatory responses, inflammation, autoimmune responses, disorders and diseases, treatable in accordance with the invention include: rheumatoid arthritis, juvenile rheumatoid arthritis, osteoarthritis, psoriatic arthritis, multiple sclerosis (MS), encephalomyelitis, myasthenia gravis, systemic lupus erythematosus (SLE), asthma, allergic asthma, autoimmune thyroiditis, atopic dermatitis, eczematous dermatitis, psoriasis, Sjögren's Syndrome, Crohn's disease, aphthous ulcer, iritis, conjunctivitis, keratoconjunctivitis, ulcerative colitis (UC), inflammatory bowel disease (IBD), cutaneous lupus erythematosus, scleroderma, vaginitis, proctitis, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, interstitial lung fibrosis, Hashimoto's thyroiditis, autoimmune polyglandular syndrome, insulin-dependent diabetes mellitus, insulin-resistant diabetes mellitus, immune-mediated infertility, autoimmune Addison's disease, pemphigus vulgaris, pemphigus foliaceus, dermatitis herpetiformis, autoimmune alopecia, vitiligo, autoimmune hemolytic anemia, autoimmune thrombocytopenic purpura, pernicious anemia, Guillain-Barre syndrome, stiff-man syndrome, acute rheumatic fever, sympathetic ophthalmia, Goodpasture's syndrome, systemic necrotizing vasculitis, antiphospholipid syndrome or an allergy, Behcet's disease, severe combined immunodeficiency (SCID), recombinase activating gene (RAG 1/2) deficiency, adenosine deaminase (ADA) deficiency, interleukin receptor common γ chain (γ_{c}) deficiency, Janus-associated kinase 3 (JAK3) deficiency and reticular dysgenesis; primary T cell immunodeficiency such as DiGeorge syndrome, Nude syndrome, T cell receptor deficiency, MHC class II deficiency, TAP-2 deficiency (MHC class I deficiency), ZAP70 tyrosine kinase deficiency and purine nucleotide phosphorylase (PNP) deficiency, antibody deficiencies, X-linked agammaglobulinemia (Bruton's tyrosine kinase deficiency), autosomal recessive agammaglobulinemia, Mu heavy chain deficiency, surrogate light chain (75/14.1) deficiency, Hyper-IgM syndrome: X-linked (CD40 ligand deficiency) or non-X-linked, Ig heavy chain gene deletion, IgA deficiency, deficiency of IgG subclasses (with or without IgA deficiency), common variable immunodeficiency (CVID), antibody deficiency with normal immunoglobulins; transient hypogammaglobulinemia of infancy, interferon γ receptor (IFNGR1, IFNGR2) deficiency, interleukin 12 or interleukin 12 receptor deficiency, immunodeficiency with thymoma, Wiskott-Aldrich syndrome (WAS protein deficiency), ataxia telangiectasia (ATM deficiency), X-linked lymphoproliferative syndrome (SH2D1A/SAP deficiency), and hyper IgE syndrome.

Non-limiting examples of adverse cardiovascular events and cardiovascular diseases treatable in accordance with the invention include coronary artery or heart disease, atherosclerosis, peripheral artery disease, cerebrovascular disease, renal artery disease, stroke, myocardial infarction (heart attack), ischemic heart failure, transient ischemic attack or brain trauma, and elevated blood cholesterol.

Methods and uses of the invention include uses and administering to or contact of a subject with Ly6C− type monocytes (or human equivalents, e.g., CD14⁺ CD16⁺ monocytes and/or CD14^{dim}CD16⁺ (CD115⁺CD11b⁺GR1)) monocytes or macrophages (cell based therapy). Accordingly, methods of the invention include administration to a subject and uses of CD14⁺ CD16⁺ and/or CD14^{dim} CD16⁺ monocytes and/or macrophages in order to achieve a desired result in a subject or to effect treatment of a subject. Such cells (monocytes or macrophages) can be administered or delivered or used, for example, to decrease, reduce, inhibit, suppress, limit or control undesirable or aberrant immune responses, inflammatory responses, or inflammation (e.g., swelling, pain, rash, headache, fever, nausea, diarrhea, bloat, lethargy, skeletal joint stiffness, stroke, pain, paralysis, vision or other sensory impairment, or damage in any cell, tissue or organ), autoimmune responses, disorders and diseases (e.g., rheumatoid arthritis, juvenile rheumatoid arthritis, osteoarthritis, psoriatic arthritis, MS, encephalomyelitis, myasthenia gravis, SLE, asthma, allergic asthma, autoimmune thyroiditis, atopic dermatitis, eczematous dermatitis, psoriasis, Sjögren's Syndrome, Crohn's disease, aphthous ulcer, iritis, conjunctivitis, keratoconjunctivitis, ulcerative colitis (UC), inflammatory bowel disease (IBD), cutaneous lupus erythematosus, scleroderma, vaginitis, proctitis, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, interstitial lung fibrosis, Hashimoto's thyroiditis, autoimmune polyglandular syndrome, insulin-dependent diabetes mellitus, insulin-resistant diabetes mellitus, immune-mediated infertility, autoimmune Addison's disease, pemphigus vulgaris, pemphigus foliaceus, dermatitis herpetiformis, autoimmune alopecia, vitiligo, autoimmune hemolytic anemia, autoimmune thrombocytopenic purpura, pernicious anemia, Guillain-Barre syndrome, stiff-man syndrome, acute rheumatic fever, sympathetic ophthalmia, Goodpasture's syndrome, systemic necrotizing vasculitis, antiphospholipid syndrome or an allergy, Behcet's disease, severe combined immunodeficiency (SCID), recombinase activating gene (RAG 1/2) deficiency, adenosine deaminase (ADA) deficiency, interleukin receptor common γ chain (γ_{c}) deficiency, Janus-associated kinase 3 (JAK3) deficiency and reticular dysgenesis; primary T cell immunodeficiency such as DiGeorge syndrome, Nude syndrome, T cell receptor deficiency, MHC class II deficiency, TAP-2 deficiency (MHC class I deficiency), ZAP70 tyrosine kinase deficiency and purine nucleotide phosphorylase (PNP) deficiency, antibody deficiencies, X-linked agammaglobulinemia (Bruton's tyrosine kinase deficiency), autosomal recessive agammaglobulinemia, Mu heavy chain deficiency, surrogate light chain (γ5/14.1) deficiency, Hyper-IgM syndrome: X-linked (CD40 ligand deficiency) or non-X-linked, Ig heavy chain gene deletion, IgA deficiency, deficiency of IgG subclasses (with or without IgA deficiency), common variable immunodeficiency (CVID), antibody deficiency with normal immunoglobulins; transient hypogammaglobulinemia of infancy, interferon γ receptor (IFNGR1, IFNGR2) deficiency, interleukin 12 or interleukin 12 receptor deficiency, immunodeficiency with thymoma, Wiskott-Aldrich syndrome (WAS protein deficiency), ataxia telangiectasia (ATM deficiency), X-linked lymphoproliferative syndrome (SH2D1A/SAP deficiency), and hyper IgE syndrome) and adverse cardiovascular events and cardiovascular diseases (e.g., coronary artery or heart disease, atherosclerosis, peripheral artery disease, cerebrovascular disease, renal artery disease, stroke, myocardial infarction (heart attack), ischemic heart failure, transient ischemic attack or brain trauma, or elevated blood cholesterol).

In various embodiments of the invention, a method results in increasing the amount Nur77 polypeptide sequence in the subject, thereby effecting treatment of the subject in accordance with the invention uses and methods. An increase in Nur77 can be achieved by introduction of Nur77 or a nucleic acid encoding Nur77, or an agonist of Nur77 that increases or stimulates expression of Nur77 (e.g., endogenous Nur77 expression). Accordingly, methods and uses of the invention include administration of Nur77 or a nucleic acid encoding Nur77, or an agonist of Nur77 that increases or stimulates expression of Nur77. Amounts desired may vary depending upon the subject, the desired effect, and the disorder or disease, or risk of disorder or disease, to be treated.

The term “contacting” means direct or indirect binding or interaction between two or more entities (e.g., between a Nur77 polypeptide sequence or Nur 77 agonist and a target). A particular example of direct interaction is binding (e.g., Nur77 agonist binding to Nur77 polypetide target). A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration, or delivery.

In methods and uses of the invention, Nur77, Nur77 agonist or Ly6C− type monocytes (or human equivalents, e.g., CD14⁺ CD16⁺ monocytes and/or CD14^{dim}CD16⁺ (CD115⁺CD11b⁺GR1⁻ monocytes) or macrophages or a composition thereof can be administered prior to, substantially contemporaneously with or following an undesirable or aberrant immune response, immune disorder, inflammatory response, or inflammation, an autoimmune response, disorder or disease, or an adverse cardiovascular event or disease, or one or more adverse symptoms, disorders, illnesses, pathologies, diseases, or complications caused by or associated with the foregoing. Thus, methods and uses of the invention may be practiced prior to (i.e. prophylaxis), concurrently with or after evidence of the response, disorder or disease begins (e.g., one or more symptoms of an undesirable or aberrant immune response, immune disorder, inflammatory response, inflammation, an autoimmune response, disorder or disease, or adverse cardiovascular event or disease), or one or more adverse symptoms, disorders, illnesses, pathologies, diseases, or complications caused by or associated with the undesirable or aberrant immune response, immune disorder, inflammatory response, inflammation, an autoimmune response, disorder or disease or an adverse cardiovascular event or disease. Administering Nur77, Nur77 agonist, Ly6C− type monocytes (or human equivalents, e.g., CD14⁺ CD16⁺ monocytes and/or CD14^{dim}CD16⁺ (CD115⁺CD11b⁺GR1⁻ monocytes) or macrophages or a composition thereof prior to, concurrently with or immediately following development of an adverse symptom may decrease, reduce, inhibit, suppress, limit or control the occurrence, frequency, severity, progression, or duration of one or more adverse symptoms, disorders, illnesses, pathologies, diseases, or complications caused by or associated with the undesirable or aberrant immune response, immune disorder, inflammatory response, inflammation or autoimmune response, disorder or disease, or the adverse cardiovascular event or disease.

In addition, administering Nur77, Nur77 agonist, Ly6C− type monocytes (or human equivalents, e.g., CD14⁺ CD16⁺ monocytes and/or CD14^{dim}CD16⁺ (CD115⁺CD11b⁺GR1⁻ monocytes) or macrophages or a composition thereof prior to, concurrently with or immediately following development of one or more adverse symptoms may decrease, reduce, inhibit, suppress, limit, control or prevent damage to cells, tissues or organs that occurs, for example, due to one or more adverse symptoms, disorders, illnesses, pathologies, diseases, or complications caused by or associated with the undesirable or aberrant immune response, immune disorder, inflammatory response, inflammation or autoimmune response, disorder or disease, or the adverse cardiovascular event or disease.

The invention provides combinations of Nur77, Nur77 agonist, CD14⁺ CD16⁺ or CD14^{dim} CD16 and/⁺ monocytes or macrophages and a second agent or drug, and methods and uses of such combinations. In one embodiment, a composition includes Nur77 and/or a Nur77 agonist and/or Ly6C− type monocytes (or human equivalents, e.g., CD14⁺ CD16⁺ monocytes and/or CD14^{dim}CD16⁺ (CD115⁺CD11b⁺GR1⁻ monocytes) or macrophages, and an anti-inflammatory agent or drug. Nur77, Nur77 agonist, Ly6C− type monocytes (or human equivalents, e.g., CD14⁺ CD16⁺ monocytes and/or CD14^{dim}CD16⁺ (CD115⁺CD11b⁺GR1⁻ monocytes) or macrophages or a composition thereof can be administered in combination with a second agent, drug or treatment, such as an immunosuppressive, anti-inflammatory, or palliative agent, drug or treatment. According to the invention, Nur77, Nur77 agonist, Ly6C− type monocytes (or human equivalents, e.g., CD14⁺ CD16⁺ monocytes and/or CD14^{dim}CD16⁺ (CD115⁺CD11b⁺GR1⁻ monocytes) or macrophages or a composition thereof can be administered prior to, substantially contemporaneously with or following administering a second agent, drug or treatment, such as an immunosuppressive, anti-inflammatory, autoimmune, cardiovascular, or palliative agent, drug or treatment.

Non-limiting examples of second agents and drugs include anti-inflammatory agents, such as steroidal and non-steroidal anti-inflammatory drugs (NSAIDs) to limit or control inflammatory symptoms. Exemplary NSAIDs include, without limitation, ibuprofen (2-(isobutylphenyl)-propionic acid); methotrexate (N-[4-(2,4 diamino 6-pteridinyl-methyl]methylamino]benzoyl)-L-glutamic acid); aspirin (acetylsalicylic acid); salicylic acid; diphenhydramine (2-(diphenylmethoxy)-N,N-dimethylethylamine hydrochloride); naproxen (2-naphthaleneacetic acid, 6-methoxy-9-methyl-, sodium salt, (−)); ketorolac (1H-Pyrrolizine-1-carboxylic acid, 2,3-dihydro-5-benzoyl-, (+-)); phenylbutazone (4-butyl-1,2-diphenyl-3,5-pyrazolidinedione); sulindac-(2)-5-fluoro-2-methyl-1-[[p-(methylsulfinyl)phenyl]methylene-]-1-H-indene-3-acetic acid; diflunisal (2′,4′,-difluoro-4-hydroxy-3-biphenylcarboxylic acid; piroxicam (4-hydroxy-2-methyl-N-2-pyridinyl-2H-1,2-benzothiazine-2-carboxamide 1,1-dioxide, an oxicam; indomethacin (1-(4-chlorobenzoyl)-5-methoxy-2-methyl-H-indole-3-acetic acid); meclofenamate sodium (N-(2,6-dichloro-m-tolyl)anthranilic acid, sodium salt, monohydrate); ketoprofen (2-(3-benzoylphenyl)-propionic acid; tolmetin sodium (sodium 1-methyl-5-(4-methylbenzoyl-1H-pyrrole-2-acetate dihydrate); diclofenac sodium (2-[(2,6-dichlorophenyl)amino]benzeneatic acid, monosodium salt); hydroxychloroquine sulphate (2-{[4-[(7-chloro-4-quinolyl)amino]pentyl]ethylamino}ethanol sulfate (1:1); penicillamine (3-mercapto-D-valine); flurbiprofen ([1,1-b]phenyl]-4-acetic acid, 2-fluoro-alphamethyl-, (+-)); cetodolac (1-8-diethyl-13,4,9, tetrahydropyrano-[3-4-13]indole-1-acetic acid; mefenamic acid (N-(2,3-xylyl)anthranilic acid; and diphenhydramine hydrochloride (2-diphenyl methoxy-N,N-di-methylethamine hydrochloride).

Immunosuppressive corticosteroids include (steroid receptor agonists) such as budesonide, prednisone, flunisolide. Anti-inflammatory agents also include flunisolide hydrofluoroalkane, estrogen, progesterone, dexamethasone and loteprednol; beta-agonists (e.g., short or long-acting) such as bambuterol, formoterol, salmeterol, albuterol; anticholinergics such as ipratropium bromide, oxitropium bromide, cromolyn and calcium-channel blocking agents; antihistamines such as terfenadine, astemizole, hydroxyzine, chlorpheniramine, tripelennamine, cetirizine, desloratadine, mizolastine, fexofenadine, olopatadine hydrochloride, norastemizole, levocetirizine, levocabastine, azelastine, ebastine and loratadine; antileukotrienes (e.g., anti-cysteinyl leukotrienes (CysLTs)) such as oxatomide, montelukast, zafirlukast and zileuton; phosphodiesterase inhibitors (e.g., PDE4 subtype) such as ibudilast, cilomilast, BAY 19-8004, theophylline (e.g., sustained-release) and other xanthine derivatives (e.g., doxofylline); thromboxane antagonists such as seratrodast, ozagrel hydrochloride and ramatroban; prostaglandin antagonists such as COX-1 and COX-2 inhibitors (e.g., celecoxib and rofecoxib), aspirin; and potassium channel openers.

Additional non-limiting examples of classes of other agents and drugs include anti-inflammatory agents that are immunomodulatory therapies, such as pro-inflammatory cytokine antagonists, such as TNFα antagonists (e.g. etanercept) and the anti-IL-6 receptor tocilizumab; immune cell antagonists, such as the B cell depleting agent rituximab and the T cell costimulation blocker abatacept, which have been used to treat rheumatoid arthritis, and antibodies that bind to cytokines, such as anti-IgE (e.g., rhuMAb-E25 omalizumab), and anti-TNFα, IFNγ, IL-1, IL-2, IL-5, IL-6, IL-9, IL-13, IL-16, and growth factors such as granulocyte/macrophage colony-stimulating factor.

Accordingly, the invention provides Nur77, Nur77 agonist, Ly6C− type monocytes (or human equivalents, e.g., CD14⁺ CD16⁺ monocytes and/or CD14^{dim}CD16⁺ (CD115⁺CD11b⁺GR1⁻ monocytes) or macrophages and combinations thereof with such agents, drugs and treatments, and methods and uses of such combinations. The invention also provides methods and uses of such Nur77, Nur77 agonists, Ly6C− type monocytes (or human equivalents, e.g., CD14⁺ CD16⁺ monocytes and/or CD14^{dim}CD16⁺ (CD115⁺CD11b⁺GR1⁻ monocytes) or macrophages which are administered prior to, substantially contemporaneously with or following administering a second agent, drug or treatment, such as an immunosuppressive, anti-inflammatory, autoimmune, cardiovascular, or palliative agent, drug or treatment.

Invention compositions, methods and uses, such as treatment methods and uses, can provide a detectable or measurable therapeutic benefit or improvement to a subject. A therapeutic benefit or improvement is any measurable or detectable, objective or subjective, transient, temporary, or longer-term benefit to the subject or improvement in the response, disorder or disease, or one or more adverse symptoms, disorders, illnesses, pathologies, diseases, or complications caused by or associated with the undesirable or aberrant response, disorder or disease.

Therapeutic benefits and improvements include, but are not limited to, decreasing, reducing, inhibiting, suppressing, limiting or controlling the occurrence, frequency, severity, progression, or duration of an adverse symptom, such as swelling, pain, rash, headache, fever, nausea, diarrhea, bloat, lethargy, skeletal joint stiffness, stroke, pain, paralysis, vision or other sensory impairment, or tissue or cell damage. Compositions, methods and uses of the invention therefore include providing a therapeutic benefit or improvement to a subject.

Compositions, methods and uses of the invention, in which a therapeutic benefit or improvement is a desired outcome, a composition such as a Nur77 polypeptide sequence or Nur77 agonist, or Ly6C− type monocytes (or human equivalents, e.g., CD14⁺ CD16⁺ monocytes and/or CD14^{dim}CD16⁺ (CD115⁺CD11b⁺GR1⁻ monocytes) or macrophages, can be administered in a sufficient or effective amount to a subject in need thereof. An “effective amount” or “sufficient amount” refers to an amount that provides, in single or multiple doses, alone or in combination, with one or more other compositions (therapeutic agents such as a drug), treatments, protocols, or therapeutic regimens agents, a detectable response of any duration of time (long or short term), an expected or desired outcome in or a benefit to a subject of any measurable or detectable degree or for any duration of time (e.g., for minutes, hours, days, months, years, or cured).

For example, a sufficient amount of a Nur77 polypeptide sequence, a Nur77 agonist, Ly6C− type monocytes (or human equivalents, e.g., CD14⁺ CD16⁺ monocytes and/or CD14^{dim}CD16⁺ (CD115⁺CD11b⁺GR1⁻ monocytes) or macrophages, or a composition thereof, is considered as having a therapeutic effect if administration results in a decreased, reduced, inhibited, or limit of the amount or frequency of therapy for treatment of an undesirable or aberrant immune response, immune disorder, inflammatory response, inflammation or autoimmune response, disorder or disease, or one or more adverse symptoms, disorders, illnesses, pathologies, diseases, or complications caused by or associated with the undesirable or aberrant immune response, immune disorder, inflammatory response, inflammation, autoimmune response, disorder or disease, or adverse cardiovasuclar event or disease.

The doses of an “effective amount” or “sufficient amount” for treatment (e.g., to provide a therapeutic benefit or improvement) typically are presumed effective to ameliorate a response, disorder or disease, or one, multiple or all adverse symptoms, consequences or complications of the response, disorder or disease, one or more adverse symptoms, disorders, illnesses, pathologies, diseases, or complications, for example, caused by or associated with an undesirable or aberrant immune response, immune disorder, inflammatory response, inflammation , autoimmune response, disorder or disease, or advserse cardiovascular event or disease to a measurable extent, although decreasing, reducing, inhibiting, suppressing, limiting or controlling progression or worsening of the response, disorder or disease, or an adverse symptom thereof, is a satisfactory outcome.

The term “ameliorate” means a detectable or measurable improvement in a subject's condition or an underlying cellular response. A detectable or measurable improvement includes a subjective or objective decrease, reduction, inhibition, suppression, limit or control in the occurrence, frequency, severity, progression, or duration of the response, disorder or disease, such as an undesirable or aberrant immune response, immune disorder, inflammatory response, inflammation, autoimmune response, disorder or disease, or an adverse cardiovascular event or disease, or one or more adverse symptoms, disorders, illnesses, pathologies, diseases, or complications caused by or associated with any of the foregoing, or an improvement in an underlying cause or a consequence of any of the foregoing, or a reversal of any of the foregoing. Such improvements can also occur at the cellular, tissue or organ level.

Treatment can therefore result in decreasing, reducing, inhibiting, suppressing, limiting, controlling or preventing a response, disorder or disease, or an associated adverse symptom or consequence, or underlying cause; decreasing, reducing, inhibiting, suppressing, limiting, controlling or preventing a progression or worsening of a response, disorder or disease, symptom or consequence, or underlying cause; or further deterioration or occurrence of one or more additional adverse symptoms of the response, disorder or disease. Thus, a successful treatment outcome can lead to a “therapeutic effect,” or “benefit” of decreasing, reducing, inhibiting, suppressing, limiting, controlling or preventing the occurrence, frequency, severity, progression, or duration of an undesirable or aberrant immune response, immune disorder, inflammatory response, inflammation, autoimmune response, disorder or disease, or adverse cardiovascular event or disease, or one or more adverse symptoms or underlying causes or consequences of the foregoing in the subject. Treatment methods affecting one or more underlying causes of the response, disorder or disease or adverse symptom are therefore considered to be beneficial. Stabilizing a response, disorder or disease, or an adverse symptom thereof, is also a successful treatment outcome.

A therapeutic benefit or improvement therefore need not be complete ablation of the response, disorder or disease, or any one, most or all adverse symptoms, complications, consequences or underlying causes associated with the response, disorder or disease. Thus, a satisfactory endpoint is achieved when there is an incremental improvement in a subject's response, disorder or disease, or a partial decrease, reduction, inhibition, suppression, limit, control or prevention in the occurrence, frequency, severity, progression, or duration, or inhibition or reversal, of the response, disorder or disease, or one or more adverse symptoms or complications or consequences or underlying causes thereof, worsening or progression of the response, disorder or disease (e.g., stabilizing one or more symptoms or complications of the response, disorder or disease), such as undesirable or aberrant immune response, immune disorder, inflammatory response, inflammation, autoimmune response, disorder or disease, or adverse cardiovascular event or disease, or one or more adverse symptoms, disorders, illnesses, pathologies, diseases, or complications caused by or associated with undesirable or aberrant immune response, immune disorder, inflammatory response, inflammation , autoimmune response, disorder or disease, or adverse cardiovascular event or disease, over a short or long duration of time (hours, days, weeks, months, etc.).

An effective amount or a sufficient amount can but need not be provided in a single administration, may require multiple administrations, and, can but need not be, administered alone or in combination with another composition (e.g., agent), treatment, protocol or therapeutic regimen. For example, the amount may be proportionally increased as indicated by the need of the subject, type, status and severity of the response, disorder, or disease treated or side effects (if any) of treatment. In addition, an effective amount or a sufficient amount need not be effective or sufficient if given in single or multiple doses without a second composition (e.g., another drug or agent), treatment, protocol or therapeutic regimen, since additional doses, amounts or duration above and beyond such doses, or additional compositions (e.g., drugs or agents), treatments, protocols or therapeutic regimens may be included in order to be considered effective or sufficient in a given subject. Amounts considered sufficient also include amounts that result in a reduction of the use of another treatment, therapeutic regimen or protocol.

An effective amount or a sufficient amount need not be effective in each and every subject treated, prophylactically or therapeutically, or a majority of treated subjects in a given group or population. An effective amount or a sufficient amount means effectiveness or sufficiency in a particular subject, or an amount presumed to be effective based upon effectiveness or sufficiency in similarly matched subjects, but not a group or the general population. As is typical for such methods and uses, some subjects will exhibit a greater response, or less or no response to a treatment method or use.

Particular non-limiting examples of therapeutic benefit or improvement for a response, disorder or disease include, for example, decreasing, reducing, inhibiting, suppressing, limiting, controlling, or preventing occurrence, frequency, severity, progression, or duration, or stabilizing or preventing a worsening or progression the response, disorder or disease of the undesirable or aberrant immune response, immune disorder, inflammatory response, inflammation or autoimmune response, disorder or disease, the adverse cardiovascular event or disease or one or more adverse symptoms thereof. Adverse symptoms caused by or associated with undesirable or aberrant immune response, immune disorder, inflammatory response, inflammation, autoimmune response, disorder or disease, or an adverse cardiovascular event or disease are disclosed herein and are known to one of skill in the art. A therapeutic benefit can also include reducing susceptibility of a subject to an undesirable or aberrant immune response, immune disorder, inflammatory response, inflammation or autoimmune response, disorder or disease, or an adverse cardiovascular event or disease; or accelerating recovery from one or more adverse symptoms, disorders, illnesses, pathologies, diseases, or complications caused by or associated with an undesirable or aberrant immune response, immune disorder, inflammatory response, inflammation or autoimmune response, disorder or disease or an adverse cardiovascular event or disease.

Effectiveness of a treatment method, such as a therapeutic benefit or improvement for a disorder or disease, can be ascertained by various methods. Such methods include, for example, scores measuring swelling, pain, rash, headache, fever, nausea, diarrhea, bloat, lethargy, skeletal joint stiffness, or tissue or cell damage. Measuring antibodies, cytokines or chemokines using various immunological assays, such as ELISA. Determining the degree of tissue or cell damage can be ascertained by CT scanning, MRI, ultrasound, molecular contrast imaging, or molecular ultrasound contrast imaging. For gastrointestinal tract, inflammation can be assessed by endoscopy (colonoscopy, gastroscopy, ERCP), for example. For inflammation of the central nervous system (CNS), cells and cytokines in spinal tap reflect inflammation, for example. CNS inflammation (MS, Parkinson's, Alzheimer's) may be reflected in the corresponding clinical function scores known in the art, for example. Peripheral nerve inflammation can include functional assessment (motor and sensor), for example.

As is typical for treatment or therapeutic methods, some subjects will exhibit greater or less response to a given treatment, therapeutic regimen or protocol. Thus, appropriate amounts will depend upon the response, disorder or disease treated (e.g., the type, status, extent or severity of undesirable or aberrant immune response, immune disorder, inflammatory response, inflammation, autoimmune response, disorder or disease or adverse cardiovascular event or disease), the therapeutic effect desired, as well as the individual subject (e.g., overall health, the bioavailability, gender, age, etc.).

The term “subject” refers to animals, typically mammalian animals, such as humans, non human primates (apes, gibbons, chimpanzees, orangutans, macaques), domestic animals (dogs and cats), farm animals (horses, cows, goats, sheep, pigs) and experimental animal (mouse, rat, rabbit, guinea pig). Subjects include animal disease models, for example, animal models of undesirable or aberrant immune response, immune disorder, inflammatory response, inflammation or autoimmune response, disorder or disease, etc. or for studying in vivo Nur77, Nur77 agonist, Ly6C− type monocytes (or human equivalents, e.g., CD14⁺ CD16⁺ monocytes and/or CD14^{dim}CD16⁺ (CD115⁺CD11b⁺GR1⁻ monocytes) or macrophages or composition thereof in a method of the invention.

Subjects appropriate for treatment include those that have (existing), have had, or are at risk of having an undesirable or aberrant immune response, immune disorder, inflammatory response, inflammation or autoimmune response, disorder or disease or an adverse cardiovascular event or disease. Such subjects include those undergoing treatment for an undesirable or aberrant immune response, immune disorder, inflammatory response, inflammation, an autoimmune response, disorder or disease, or an adverse cardiovascular event or disease as well as those who have had or have undergone treatment or therapy for an undesirable or aberrant immune response, immune disorder, inflammatory response, inflammation, an autoimmune response, disorder or disease, or an adverse cardiovascular event or disease. Specific non-limiting examples include subjects that have had or are at risk for an undesirable or aberrant immune response, immune disorder, inflammatory response, inflammation, an autoimmune response, disorder or disease, or an adverse cardiovascular event or disease.

Candidate subjects also include those that have or are at increased risk of an undesirable or aberrant immune response, immune disorder, inflammatory response, or inflammation, an autoimmune response, disorder or disease, or an adverse cardiovascular event or disease. A candidate subject, for example, has an undesirable or aberrant immune response, immune disorder, inflammatory response, or inflammation, an autoimmune response, disorder or disease, or adverse cardiovascular event or disease, or is being treated with a therapy or drug for treatment of an undesirable or aberrant immune response, immune disorder, inflammatory response, or inflammation, an autoimmune response, disorder or disease or adverse cardiovascular event or disease. Candidate subjects further include subjects that would benefit from a treatment or are in need of treatment for an undesirable or aberrant immune response, immune disorder, inflammatory response, or inflammation, an autoimmune response, disorder or disease, an adverse cardiovascular event or disease or an adverse cardiovascular event or disease.

“At risk” subjects typically have increased risk factors for an undesirable or aberrant immune response, immune disorder, inflammatory response, inflammation, an autoimmune response, disorder or disease, or an adverse cardiovascular event or disease. Particular subjects at risk include subjects that have in the past had an undesirable or aberrant immune response, immune disorder, inflammatory response, or inflammation, an autoimmune response, disorder or disease, or an adverse cardiovascular event or disease. Particular subjects at risk also include subjects prescribed or candidates for treatment or therapy of an undesirable or aberrant immune response, immune disorder, inflammatory response, inflammation, an autoimmune response, disorder or disease or an adverse cardiovascular event or disease.

Subjects also include a subject diagnosed for an adverse cardiovascular event or cardiovascular disease (for example, coronary artery disease, peripheral artery disease, cerebrovascular disease, or renal artery disease). Additional subjects include those that have had a stroke, myocardial infarction (heart attack), ischemic heart failure, transient ischemic attack or brain trauma. Further subjects include those that have had a treatment or therapy for atherosclerotic plaque formation, elevated blood cholesterol or a cardiovascular disease.

Subjects can optionally be excluded. For example, subjects that have been diagnosed for cancer, treated for cancer, or are in remission from cancer can optionally be excluded from a method or use of the invention.

Nur77 polypeptide sequences, Nur77 agonists, Ly6C− type monocytes (or human equivalents, e.g., CD14⁺ CD16⁺ monocytes and/or CD14^{dim}CD16⁺ (CD115⁺CD11b⁺GR1⁻ monocytes) or macrophages, and compositions thereof may be contacted or provided in vitro, ex vivo or administered or delivered in vivo. Nur77 polypeptide sequences, Nur77 agonists, Ly6C− type monocytes (or human equivalents, e.g., CD14⁺ CD16⁺ monocytes and/or CD14^{dim}CD16⁺ (CD115⁺CD11b⁺GR1⁻ monocytes) or macrophages, and compositions thereof can be administered or delivered to provide the intended effect, as single or multiple dosages, for example, in an effective or sufficient amount. Exemplary doses range from about 25-250, 250-500, 500-1000, 1000-2500 or 2500-5000, 5000-25,000, 5000-50,000 pg/kg; from about 50-500, 500-5000, 5000-25,000 or 25,000-50,000 ng/kg; and from about 25-250, 250-500, 500-1000, 1000-2500 or 2500-5000, 5000-25,000, 5000-50,000 mg/kg, on consecutive days, alternating days or intermittently.

Single or multiple doses can be administered on the same or consecutive days, alternating days or intermittently. For example, a Nur77 polypeptide sequence, Nur77 agonist, or Ly6C− type monocytes (or human equivalents, e.g., CD14⁺ CD16⁺ monocytes and/or CD14^{dim}CD16⁺ (CD115⁺CD11b⁺GR1⁻ monocytes) or macrophages, or composition thereof can be administered one, two, three, four or more times daily, on alternating days, bi-weekly, weekly, monthly, bi-monthly, or annually. Nur77 polypeptide sequences, Nur77 agonists, Ly6C− type monocytes (or human equivalents, e.g., CD14⁺ CD16⁺ monocytes and/or CD14^{dim}CD16⁺ (CD115⁺CD11b⁺GR1⁻ monocytes) or macrophages, and compositions thereof can be administered for any appropriate duration, for example, for period of 1 hour, or less, e.g., 30 minutes or less, 15 minutes or less, 5 minutes or less, or 1 minute or less.

Nur77 polypeptide sequences, Nur77 agonists, Ly6C− type monocytes (or human equivalents, e.g., CD14⁺ CD16⁺ monocytes and/or CD14^{dim}CD16⁺ (CD115⁺CD11b⁺GR1⁻ monocytes) or macrophages and compositions thereof can be administered to a subject and methods may be practiced substantially contemporaneously with, or within about 1-60 minutes, hours (e.g., within 1, 2, 3, 4 or 5 hours), or days of the onset of an undesirable or aberrant immune response, immune disorder, inflammatory response, inflammation, autoimmune response, disorder or disease or adverse cardiovascular event or disease.

Nur77 polypeptide sequences, Nur77 agonists, Ly6C− type monocytes (or human equivalents, e.g., CD14⁺ CD16⁺ monocytes and/or CD14^{dim}CD16⁺ (CD115⁺CD11b⁺GR1⁻ monocytes) or macrophages and compositions thereof can be administered and methods and uses may be practiced via systemic, regional or local administration, by any route. For example, a Nur77 polypeptide sequence, Nur77 agonist, Ly6C− type monocytes (or human equivalents, e.g., CD14⁺ CD16⁺ monocytes and/or CD14^{dim}CD16⁺ (CD115⁺CD11b⁺GR1⁻ monocytes) or macrophages or composition thereof may be administered systemically, regionally or locally (e.g., into a region or site of inflammation) via injection, via infusion, by catheter, enema, intravenously, intraarterially, orally (e.g., ingestion or intranasal or inhalation), intramuscularly, intraperitoneally, intradermally, subcutaneously, intracavity, intrarectally, intracranially, topically, transdermally, optically, parenterally, e.g. transmucosally. Methods and uses of the invention including pharmaceutical formulations can be administered via a (micro) encapsulated delivery system or packaged into an implant for administration.

Nur77 polypeptide sequences, Nur77 agonists, Ly6C− type monocytes (or human equivalents, e.g., CD14⁺ CD16⁺ monocytes and/or CD14^{dim}CD16⁺ (CD115⁺CD11b⁺GR1⁻ monocytes) or macrophages and compositions thereof including combinations of Nur77 polypeptide sequence, and/or Nur77 agonist, and/or Ly6C− type monocytes (or human equivalents, e.g., CD14⁺ CD16⁺ monocytes and/or CD14^{dim}CD16⁺ (CD115⁺CD11b⁺GR1⁻ monocytes) or macrophages with other drugs or agents, and methods and uses also include pharmaceutical compositions, which refer to “pharmaceutically acceptable” and “physiologically acceptable” carriers, diluents or excipients. As used herein, the term “pharmaceutically acceptable” and “physiologically acceptable,” when referring to carriers, diluents or excipients includes solvents (aqueous or non-aqueous), detergents, solutions, emulsions, dispersion media, coatings, isotonic and absorption promoting or delaying agents, compatible with pharmaceutical administration and with the other components of the formulation. Such formulations include liquids (e.g., water and saline), such as an emulsion, suspension, syrup or elixir, and solid forms such as a tablet (coated or uncoated), capsule (hard or soft), powder, granule, crystal, or microbead.

Pharmaceutical compositions can be formulated to be compatible with a particular route of administration. Compositions for parenteral, intradermal, or subcutaneous administration can include a sterile diluent, such as water, saline solution, fixed oils, polyethylene glycols, glycerine, propylene glycol or other synthetic solvents. The preparation may contain one or more preservatives to prevent microorganism growth (e.g., antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose).

Pharmaceutical compositions for injection include sterile aqueous solutions

(where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). The carrier can be a solvent or dispersion medium containing, for example, water, ethanol, polyol (e.g., glycerol, propylene glycol, and polyetheylene glycol), and suitable mixtures thereof. Fluidity can be maintained, for example, by the use of a coating such as lecithin, or by the use of surfactants. Antibacterial and antifungal agents include, for example, parabens, chlorobutanol, phenol, ascorbic acid and thimerosal. Including an agent that delays absorption, for example, aluminum monostearate and gelatin can prolong absorption of injectable compositions.

For transmucosal or transdermal administration, penetrants appropriate to the barrier to be permeated are used in the formulation. Such penetrants are known in the art, and include, for example, for transmucosal administration, detergents, bile salts, and fusidic acid derivatives. Transmucosal administration can be accomplished through the use of nasal sprays, inhalation devices (e.g., aspirators) or suppositories. For transdermal administration, the active compounds are formulated into ointments, salves, gels, creams or patches.

Additional pharmaceutical formulations and delivery systems are known in the art and are applicable in the methods of the invention (see, e.g., Remington's Pharmaceutical Sciences (1990) 18th ed., Mack Publishing Co., Easton, Pa.; The Merck Index (1996) 12th ed., Merck Publishing Group, Whitehouse, N.J.; Pharmaceutical Principles of Solid Dosage Forms, Technonic Publishing Co., Inc., Lancaster, Pa., (1993); and Poznansky, et al., Drug Delivery Systems, R. L. Juliano, ed., Oxford, N.Y. (1980), pp. 253-315).

The Nur77 polypeptide sequences, Nur77 agonists, Ly6C− type monocytes (or human equivalents, e.g., CD14⁺ CD16⁺ monocytes and/or CD14^{dim}CD16⁺ (CD115⁺CD11b⁺GR1⁻ monocytes) or macrophages and compositions thereof used in accordance with the invention, including proteins, treatments, therapies, agents, drugs and pharmaceutical formulations can be packaged in dosage unit form for ease of administration and uniformity of dosage. “Dosage unit form” as used herein refers to physically discrete units suited as unitary dosages treatment; each unit contains a quantity of the composition in association with the carrier, excipient, diluent, or vehicle calculated to produce the desired treatment or therapeutic (e.g., beneficial) effect. The unit dosage forms will depend on a variety of factors including, but not necessarily limited to, the particular composition employed, the effect to be achieved, and the pharmacodynamics and pharmacogenomics of the subject to be treated.

The invention provides kits including compositions of the invention (e.g., Nur77 polypeptide sequence, Nur77 agonists, monocytes and/or macrophages, etc.), combination compositions and pharmaceutical formulations thereof, packaged into suitable packaging material. Kits can be used to practice various methods and for various uses.

A kit typically includes a label or packaging insert including a description of the components or instructions for use in vitro, in vivo, or ex vivo, of the components therein. A kit can contain a collection of such components, e.g., a Nur77 polypeptide sequence, Nur77 agonist, Ly6C− type monocytes (or human equivalents, e.g., CD14⁺ CD16⁺ monocytes and/or CD14^{dim}CD16⁺ (CD115⁺CD11b⁺GR1⁻ monocytes) or macrophages alone, or in combination with another therapeutically useful composition (e.g., anti-inflammatory, anti-autoimmune or anti-adverse cardiovascular event or disease drug or agent).

The term “packaging material” refers to a physical structure housing the components of the kit. The packaging material can maintain the components sterilely, and can be made of material commonly used for such purposes (e.g., paper, corrugated fiber, glass, plastic, foil, ampules, vials, tubes, etc.).

Kits of the invention can include labels or inserts. Labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, such as a disk (e.g., hard disk), optical disk such as CD- or DVD-ROM/RAM, DVD, MP3, magnetic tape, or an electrical storage media such as RAM and ROM or hybrids of these such as magnetic/optical storage media, FLASH media or memory type cards.

Labels or inserts can include identifying information of one or more components therein, dose amounts, clinical pharmacology of the active ingredient(s) including mechanism of action, pharmacokinetics and pharmacodynamics. Labels or inserts can include information identifying manufacturer information, lot numbers, manufacturer location and date.

Labels or inserts can include information on a response, disorder, or disease, or adverse symptom, for which a kit component may be used. Labels or inserts can include instructions for the clinician or for a subject for using one or more of the kit components in a method, treatment protocol or therapeutic regimen, or use. Instructions can include dosage amounts, frequency or duration, and instructions for practicing any of the methods, treatment protocols or therapeutic regimes and uses set forth herein. Exemplary instructions include, instructions for treating an undesirable or aberrant immune response, immune disorder, inflammatory response, inflammation, an autoimmune response, disorder or disease or an adverse cardiovascular event or disease. Kits of the invention therefore can additionally include labels or instructions for practicing any of the methods of the invention described herein including treatment methods.

Labels or inserts can include information on any benefit that a component may provide, such as a prophylactic or therapeutic benefit. Labels or inserts can include information on potential adverse side effects, such as warnings to the subject or clinician regarding situations where it would not be appropriate to use a particular composition. Adverse side effects could also occur when the subject has, will be or is currently taking one or more other medications that may be incompatible with the composition, or the subject has, will be or is currently undergoing another treatment protocol or therapeutic regimen which would be incompatible with the composition and, therefore, instructions could include information regarding such incompatibilities.

Invention kits can additionally include other components. Each component of the kit can be enclosed within an individual container and all of the various containers can be within a single package. Invention kits can be designed for cold storage. Invention kits can further be designed to contain Nur77 polypeptide sequences, Nur77 agonists, Ly6C− type monocytes (or human equivalents, e.g., CD14⁺ CD16⁺ monocytes and/or CD14^{dim}CD16⁺ (CD115⁺CD11b⁺GR1⁻ monocytes) or macrophages and compositions thereof.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All applications, publications, patents and other references, GenBank citations and ATCC citations cited herein are incorporated by reference in their entirety. In case of conflict, the specification, including definitions, will control.

As used herein, the singular forms “a”, “and,” and “the” include plural referents unless the context clearly indicates otherwise. Thus, for example, reference to “a Nur77 polypeptide sequence” or a “Nur77 agonist” includes a plurality of such Nur77 polypeptide sequences (or subsequences or variants thereof) or agonists, and reference to “a Nur77 polypeptide activity or function” can include reference to one or more Nur77 polypeptide activities or functions, and so forth.

As used herein, numerical values are often presented in a range format throughout this document. The use of a range format is merely for convenience and brevity and should not be construed as an inflexible limitation on the scope of the invention. Accordingly, the use of a range expressly includes all possible subranges, all individual numerical values within that range, and all numerical values or numerical ranges including integers within such ranges and fractions of the values or the integers within ranges unless the context clearly indicates otherwise. This construction applies regardless of the breadth of the range and in all contexts throughout this document. Thus, for example, reference to a range of 90-100% includes 91-99%, 92-98%, 93-95%, 91-98%, 91-97%, 91-96%, 91-95%, 91-94%, 91-93%, and so forth. Reference to a range of 90-100% also includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth.

In addition, reference to a range of 2-10 (e.g., amino acids) includes 2, 3, 4, 5, 6, 7, 8, 9, 10, as well as 1.1, 1.2, 1.3, 1.4, 1.5, etc., 2.1, 2.2, 2.3, 2.4, 2.5, etc., and any numerical range within such a ranges, such as 2-3, 2-4, 2-6, 3-6, 3-7, 4-8, 5-9, 5-10, etc. In a further example, reference to a range of 2-10, 10-20, 20-30, 30-40, 40-50, 50-60, 60-75, 75-100 includes any numerical value or range within or encompassing such values.

As also used herein a series of ranges are disclosed throughout this document. The use of a series of ranges includes combinations of the upper and lower ranges to provide another range. This construction applies regardless of the breadth of the range and in all contexts throughout this document. Thus, for example, reference to a series of ranges such as 2-10, 10-20, 20-30, 30-40, 40-50, 50-60, 60-75, 75-100, includes ranges such as 2-20, 2-30, 5-20, 5-30, 5-40, 5-50, 5-60, 10-30, 10-40, 10-50, and 20-40, 20-30, 20-40, 20-50, 30-50, 30-60, 30-100, and 40-60, 40-70, 40-100, 50-75, 50-100, 60-100, and so forth.

The invention is generally disclosed herein using affirmative language to describe the numerous embodiments. The invention also specifically includes embodiments in which particular subject matter is excluded, in full or in part, such as substances or materials, method steps and conditions, protocols, procedures, assays or analysis. Thus, even though the invention is generally not expressed herein in terms of what the invention does not include, aspects that are not expressly included in the invention are nevertheless disclosed herein.

A number of embodiments of the invention have been described. Nevertheless, it will be understood that various modifications may be made without departing from the spirit and scope of the invention. Accordingly, the following examples are intended to illustrate but not limit the scope of invention described in the claims.

### EXAMPLES

### Example 1

This example includes a description of various materials and methods.

### Mice

C57BL/6 (stock no. 000664) mice (BL6) were purchased from The Jackson Laboratory (Bar Harbor, Me.), B6.5JL-Ptprca/BoyAiTac (stock no. 004007) mice were purchased from Taconic Farms (Germantown, N.Y.) and Nur77−/− mice (Lee et al., Science 269:532 (1995)) on a C57BL/6J background were bred in-house. Mice were fed a standard rodent chow diet and housed in microisolator cages in a pathogen-free facility. All studies followed Animal Care and Use Committee guidelines, and approval for use of rodents was obtained according to criteria outlined in “Guide for the Care and Use of Laboratory Animals” from the National Institutes of Health. Mice were sacraficed using CO₂.

### Flow Cytometry

Spleens were excised and pushed through a 70-μm strainer, bone marrow cells from both femurs and tibias were collected by centrifugation, and blood was drawn via cardiac puncture with an EDTA coated syringe. All samples were collected in DPBS with 2 mM EDTA and maintained on ice during staining and analysis. Red blood cells were lysed in lysis buffer (Biolegend, San Diego, Calif.) according to the manufactures protocol.

Two to four×10⁶ cells were resuspended in 100 μl staining buffer (1% BSA, 0.1% sodium azide in PBS). Cells were blocked with Fcγ receptor for 15 min and stained for surface antigens with flow cytometry Abs for 30 min at 4° C. A fixable LIVE/DEAD stain (Invitrogen) were used to measure viability, and FSC/SSC parameters were used to excluded doublets from analysis. Antibodies used for flow cytometry are listed in Table 2. Cells were run immediately on a LSRII flow cytometer (BD Biosciences, San Jose, Calif.), and data analyses were performed with FlowJo software (Tree Star, Ashland, Oreg.).

TABLE 2

| | | | Secondary (if not |
|---|---|---|---|
| Antibody | Company | Clone | directly conjugated) |
| CD49b | eBioscience | DX5 | |
| CD3e | BD Pharmingen | 145-2C11 | |
| NK1.1 | eBioscience | PK136 | |
| CD19 | eBioscience | 1D3 | |
| B220 | eBioscience | RA3-6B2 | |
| Ly6G | Biolegend | 1A8 | |
| CD11c | eBioscience | N418 | |
| CD115 | Biolegend | AFS98 | |
| CD11b | BD Pharmingen | M1/70 | |
| LY6C | BD Biosciences | AL-21 | |
| CD45.1 | Biolegend | A20 | |
| CD45.2 | Biolegend | 104 | |
| CX3CR1 | Santa Cruz | T-20 | anti-goat IgG-FITC |
| | | | (SC-2777) SC |
| CCR2 | (Mack et al., | MC21 | biotin anti-rat IgG |
| | 2001) | | (RG7/11.1) BD, |
| | | | streptavidin- |
| | | | APC (554067) BD |
| Lineage | BD Pharmingen | CD3e | |
| Cocktail | | (145-2C11), | |
| | | CD11b | |
| | | (M1/70), | |
| | | B220 | |
| | | (RA3-6B2), | |
| | | Ly-76 | |
| | | (TER-119), | |
| | | Ly6C/G | |
| | | (RB6-8C5) | |
| CD117 | eBioscience | 2B8 | |
| SCA-1 | Biolegend | E13-161.7 | |
| (Ly6A/E) | | | |
| CD34 | BD Biosciences | RAM34 | |
| LIVE/DEAD | Invitrogen | | |
| Fixable Yellow | | | |
| Dead Cell Stain | | | |
| Nur77 | BD Biosciences | 12.14 | anti-mouse |
| | | | IgG-FITC |
| | | | (554088) BD |
| CD11a | BD Biosciences | M17/4 | |
| (LFA-1) | | | |
| Active | BD Biosciences | C92-605 | |
| Caspase-3 | | | |
| p65 (pS529) | BD Biosciences | K10-895.12.50 | |

For additional intracellular staining, cells were fixed and permeablized using the Cytofix/Cytoperm kit from BD Bioscience. Directly conjugated fluorescent antibodies were stained for 30 min at 4° C. Indirect antibodies were stained for 1 hr at 4° C., washed two times and then stained for 30 min at 4° C. with secondary antibody. CCR2 was stained as previously described by Mack et. al. J Immunol 166:4697 (2001). The absolute numbers of cells were calculated by multiplying the percentage of live cells in individual subsets by the total cell count before staining. Calculations of percentages were based on live cells as determined by FSC/SSC and viability analysis. Cell fluorescence was determined using a FACSCalibur flow cytometer (BD Biosciences) and analyzed with FlowJo software (version 9.2). The mean fluorescence intensity was quantified, and the expression relative to B6 was calculated.

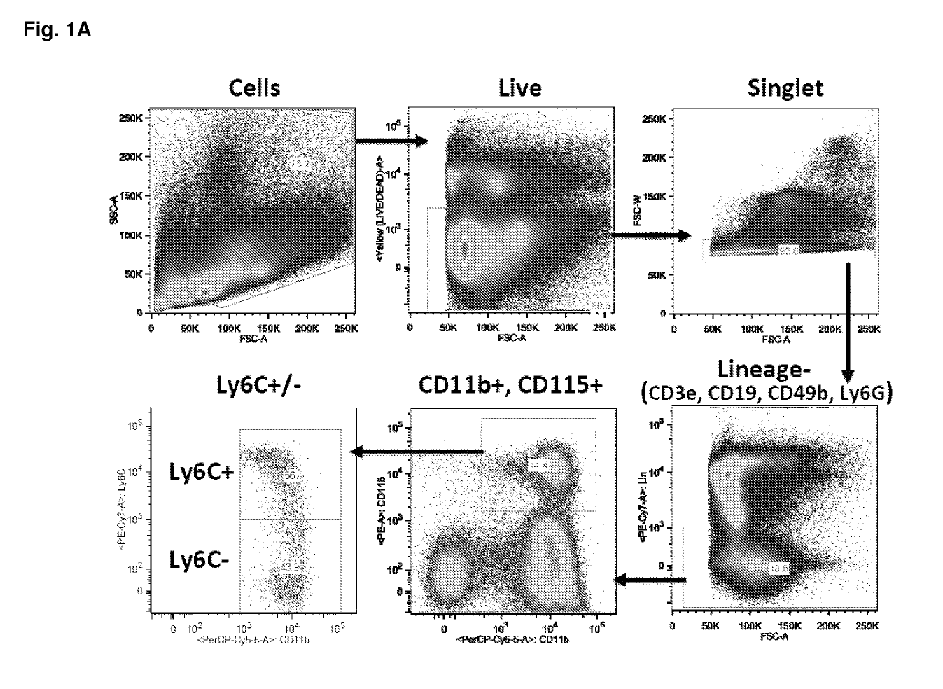

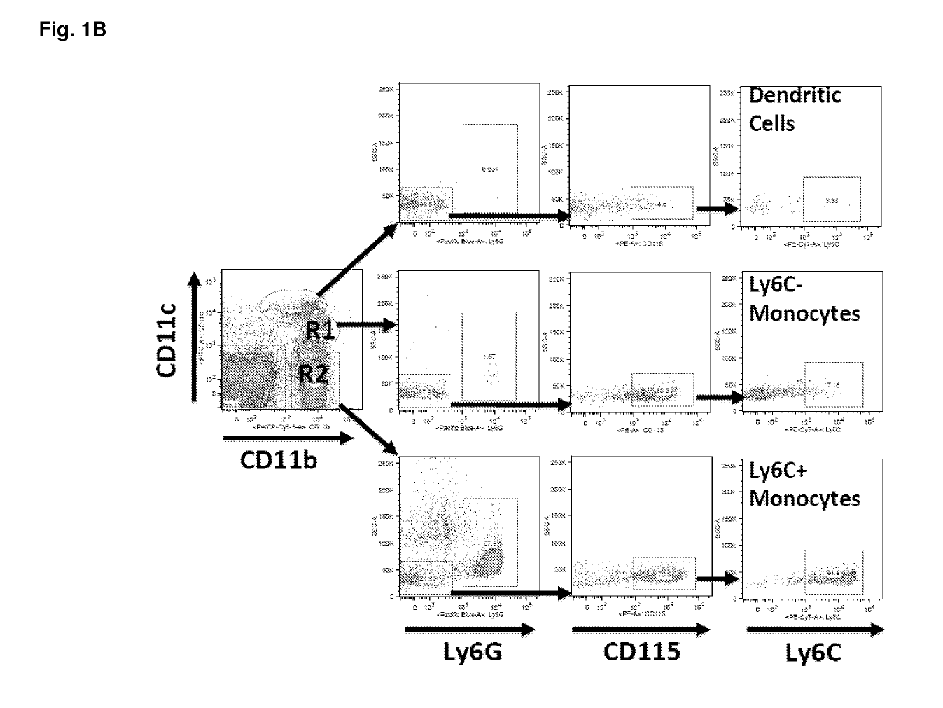

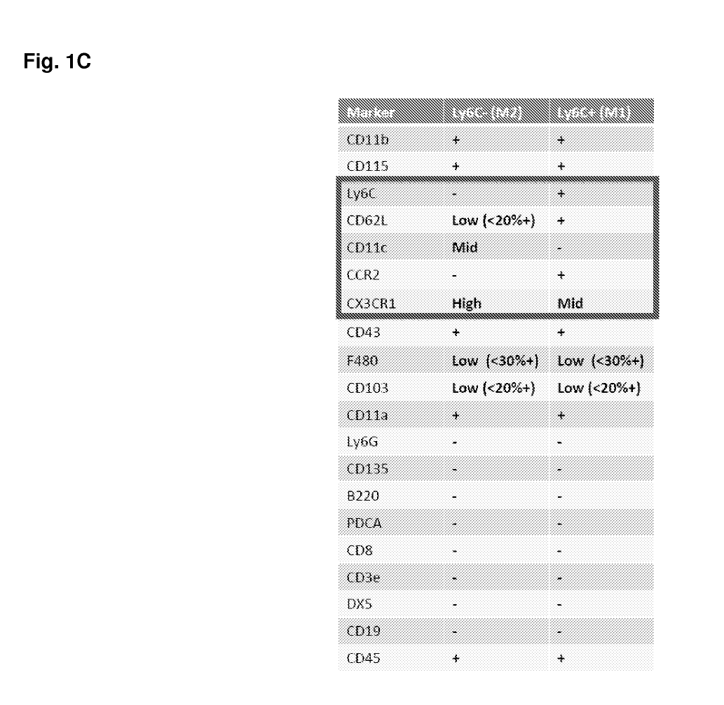

Monocytes were identified as described below (see, also, FIG. 1 description). Hematopoietic stem cells (HSC) were identified as Lin− CD117+ Sca-1+, Common Myeloid Precursors (CMP) as Lin− CD117+ Sca-1− CD34+; Macrophage Dendritic Precursors (MDP) as Lin− CD115+ CD117+; dendritic cells (DC) as CD11c^{High}; Granulocytes as Ly6G+; natural killer T cells (NKT) as CD49b+; B cells as CD19+; and T cells as CD3e+.

### Fluorescent Activated Cell Sorting (FACS)

Prior to sorting bone marrow, monocytes and stem cell populations were enriched by negative selection using Miltenyi Biotec (Auburn, Calif.) isolation kits using the manufacturer's protocol (CD19, CD4, CD8, Ter119 custom cocktail for monocytes, and lineage cell depletion kit #130090858 for stem cells). Cells were then stained for surface antigens as described above and sorted using a FACSAria cell sorter (BD Biosciences). Approximately 5−10×10⁴ events were collected for mRNA samples. Purity of monocyte and stem cell populations were verified by cytospin preps of sorted cells, followed by staining with HEMA 3 (Fisher Scientific) and microscopic analysis.

Quantitative Real-Time PCR (qRT-PCR)

Total cellular RNA was collected from FACS isolated monocyte and stem cell populations using a Qiagen RNeasy Plus Micro Kit (Valencia, Calif.) following the manufacturer's protocol. RNA purity and quantity was measured using a nanodrop spectrophotometer (Thermo Scientific). Approximately 500 nanograms of RNA was used to synthesize cDNA using an Iscript cDNA synthesis kit (Bio-Rad, Hercules, Calif.). Total cDNA was diluted 1:20 in H₂O, and 9 μl were used for each real-time condition using a Bio-Rad MyIQ single-color real-time PCR detection system and TaqMan Gene Expression Mastermix. TaqMan primers used for analysis are listed in Table 3. Data were analyzed and presented on the basis of the relative expression method (Livak and Schmittgen, 2001). The formula for this calculation is as follows: relative expression=2−(^{SΔCt-CΔC t}) where ΔCₜ is the difference in the threshold cycle between the gene of interest and the housekeeping gene (GAPDH), S is the Nur77−/− mouse, and C is the C57BL/6J control mouse.

TABLE 3
Taqman Primers

| Assay ID | Gene Symbol |
|---|---|
| 4352932-0912031 | Gapdh |
| Mm01300401_m1 | Nr4a1 (Nur77) |
| Mm00443060_m1 | Nr4a2 (Nurr1) |
| Mm00450074_m1 | Nr4a3 (NOR-1) |
| Mm00477798_m1 | Nfkbia (I κ B α) |
| Mm00772472_m1 | Cdc2a (Cdk1) |
| Mm00438064_m1 | Ccna2 (Cyclin A2) |
| Mm00438070_m1 | Ccnd2 (Cyclin D2) |
| Mm00624964_m1 | E2F2 |
| Mm00438354_m1 | cx3cr1 |
| Mm00492781_s1 | Junb |
| Mm00843434_s1 | Cebpb |
| Mm00488140_m1 | Sfpil (Pu.1) |

### Bone Marrow and MDP Transplantation Studies.

Recipient mice (BL6 or Nur77−/−) were irradiated in two doses of 550 rads each, for a total of 1100 rads, 4 h apart. Bone marrow cells from both femurs and tibias of donor mice (BL6 or Nur77−/−) were harvested under sterile conditions. Approximately 50 million nucleated bone marrow cells were obtained from each donor mouse. Bones were centrifugated to collect marrow, and then washed and resuspended for injection in DPBS. Approximately 5 million unfractionated bone marrow cells in 200 μl of media were delivered retro-orbitaly to each recipient mouse. Recipient mice were housed in a barrier facility under pathogen-free conditions before and after bone marrow transplantation. After bone marrow transplantation, the mice were maintained on autoclaved acidified water with antibiotics (Trimethoprim/Sulfa) and fed autoclaved food. Mice were used for stuides after 6 weeks of bone marrow reconstitution.

The 45.1 antigen in the B6.5JL-Ptprca/BoyAiTac mice (BL6 45.1), and the 45.2 antigen in the Nur77−/− and C57BL/6 (BL6 45.2) mice were used to track cells in mixed chimeric bone marrow transplant mice. For the mixed chimeric transplants 2.5 million cells from Nur77−/− mice (45.2) and 2.5 million cells from B6.5JL-Ptprca/BoyAiTac mice (BL6 45.1) were mixed 1:1 for reconstitution into recipients (BL6 45.1 or BL6 45.2) as described above. For MDP chimeras, approximately 5×10³ cells were reconstituted from each donor (Nur77−/−45.2 mice and BL6 45.1 mice) in a 1:1 ratio into recipients (BL6 45.1 or BL6 45.2) recipients. MDP transplants were done as described above, except that MDP recipients received only one 600 RAD dose of radiation, and were analyzed after only 7 days of reconstitution.

### Annexin V Analysis of Apoptotic Cells

Annexin V and propidium iodide were used to identify apoptotic and dead cell populations by flow cytometry. Bone marrow, blood and spleen cells were stained for annexin V (Invitrogen) and propidium iodide (Invitrogen) according to the manufacturers' protocols, after surface staining for monocye or stem cell populations.

### Cell Cycle Analysis

For cell cycle analysis, monocyte populations in the bone marrow were first stained for identifying surface antigens, and then fixed and permeablized using the Cytofix/Cytoperm kit from BD Bioscience. Permeablized cells were then stained with 50 μg/ml propidium iodide (Invitrogen) for 30 minutes at room temperature followed by one wash and resuspension with flow buffer. Cells were then analyzed on a linear scale for DNA content and then gated and analyzed for cells in G1/0, S and G2 phase of the cell cycle using FlowJo software.

### Immunofluorescence

Cytospin preparations of bone marrow monocytes sorted from BL6 and Nur77−/− mice by FACS were immunofluorescently stained for cleaved (active) caspase 3, p65 (NFκB), and DAPI. Cytospin preps were dried overnight, fixed with 4% paraformaldehyde for 20 min, washed in PBS, permeablized in 0.1% citrate 0.1% Triton X solution for 5 min, washed 3 times in PBS, blocked in 5% donkey serum with 0.2% Triton X in PBS for 60 min, and then incubated with primary antibody diluted in PBS with 0.2% Triton X and 1% BSA overnight at 4° C. The following day, slides are then washed 3 times in PBS and incubated for 1 hour at room temperature with fluorescent secondary antibodies at a 1:1000 dilution, slides were then washed 3 more times in PBS and mounted with ProlongGold with DAPI (Invitrogen). A 1:500 dilution of rabbit anti-mouse cleaved caspase-3 (Asp175) antibody from Cell Signaling (#9661) was used, followed by staining with a donkey anti-rabbit Alexa488 fluorescently conjugated secondary antibody (Invitrogen). A 1:33 dilution of mouse anti-mouse p65 (NFκB) antibody from Santa Cruz Biotechnology (sc-8008) was used followed by staining with a donkey anti-mouse Alexa594 fluorescently conjugated secondary antibody (Invitrogen). Images were acquired using an Olympus FV 10i confocal microscope and analyzed using Imaris software (Bitplane). The % of p65 fluorescence overlapping with DAPI staining was quantified in BL6 and Nur77−/− Ly6C− monocytes from the bone marrow.

### Analysis of Patrolling Cells

CD45.1 mice were irradiated (9.5 Gy) and 3 hours later injected with 2×10⁷ bone marrow cells from either C57BL6 mice (CD45.2) or Nur77−/− mice (CD45.2) in 150 ml PBS i.v. via tail vein. Before and after the procedure the mice where maintained in a sterile environment and given water containing an antimicrobial (Baytril; Bayer). 6 weeks later blood samples were obtained via tail vein and the mice were assessed for full chimerism and monocyte subset phenotype by flow cytometry. Following blood sampling, mice were anaesthetised with Ketamine/Xylazine/ACP (i.p.) and injected i.v. with 10 mg PE conjugated anti-mouse CD11b (M1/70; Becton Dickinson). Blood vessels in the ear were imaged through a glass cover slip using an inverted confocal microscope (SP5) 561 nm DPSS laser line and ×20 0.7 N. A. water immersion objective. Both the blood vessels (circulating Ab) and CD11b⁺ cells (bound Ab) were visualized over 1 hour in several fields per animal. These data were deconvolved (LSCM blind algorithm; Autoquant X2, Media Cybernetics) and visualized and analyzed using Imaris software (Bitplane). CD11b⁺ cells in the blood vessels were tracked using the autoregressive motion algorithm and tracks were filtered for a minimum track length of 30 mm from their origin and a minimum track duration of 3 mins, then manually assessed and edited for track continuity. For each field the tracks were translated to a common origin in space to allow their comparison (number, direction, displacement). These tracks were then counted and the number (i.e. the number of patrolling cells) plotted per field.

### Statistical Analysis

Data for all studies were analyzed using Prism software (GraphPad, San Diego, Calif.). Unpaired t tests and two-way ANOVA were used to compare groups. All studies were repeated at least 3 times with 3 or more individuals per group. Data are graphically represented as mean±SEM. A p value <0.05 was considered significant.

### Example 2

This example shows data indicating the loss of mature monocytes in Nur77−/− mice.

Monocytes were identified as live cells with low side scatter, and positive for CD115 and CD11b (FIG. 1A). This monocyte population is distinct from CD11c^{hi} DCs identified as CD11c^{hi}, CD11b⁺, CD115−, CD3e−, CD19−, CD49b−, Ly6G− (FIGS. 1B and 1C). The data reveal that Nur77−/− mice have almost a complete absence of Ly6C− monocytes.

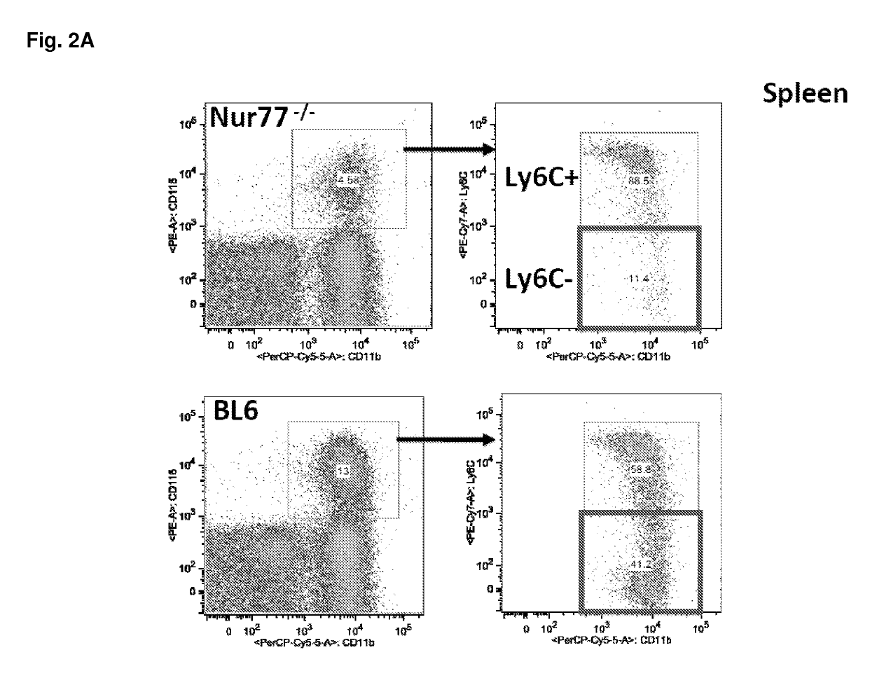

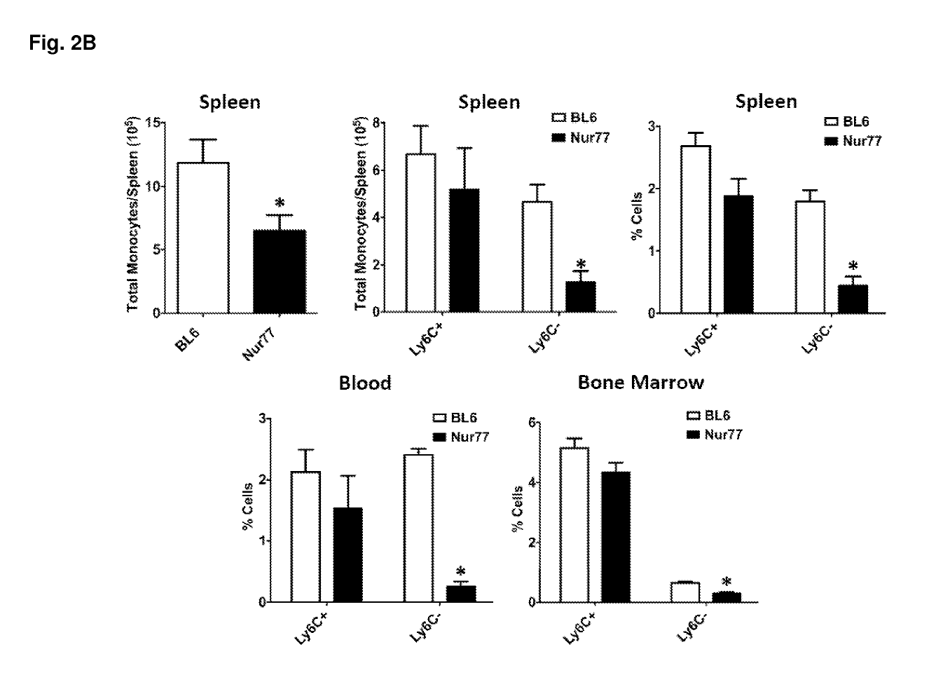

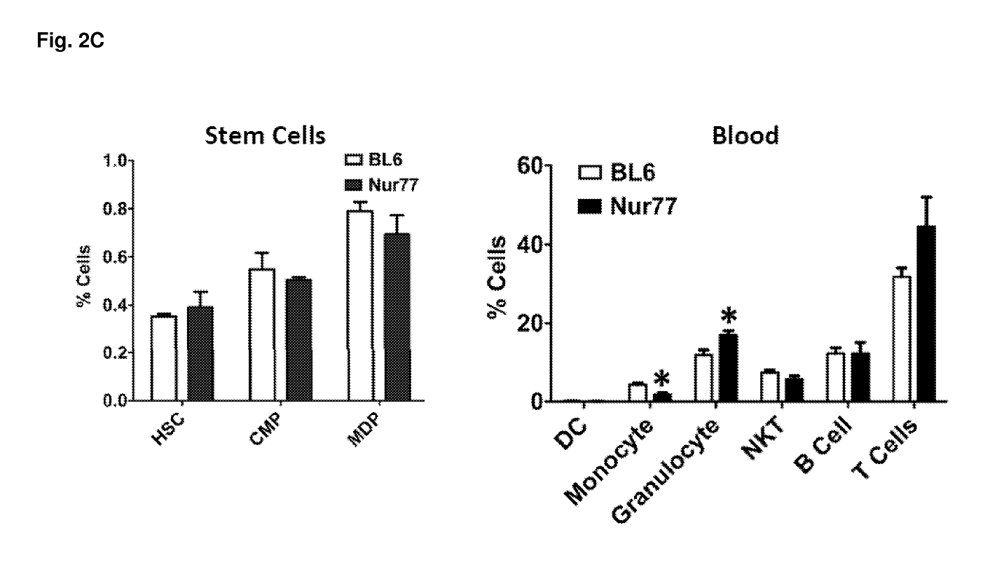

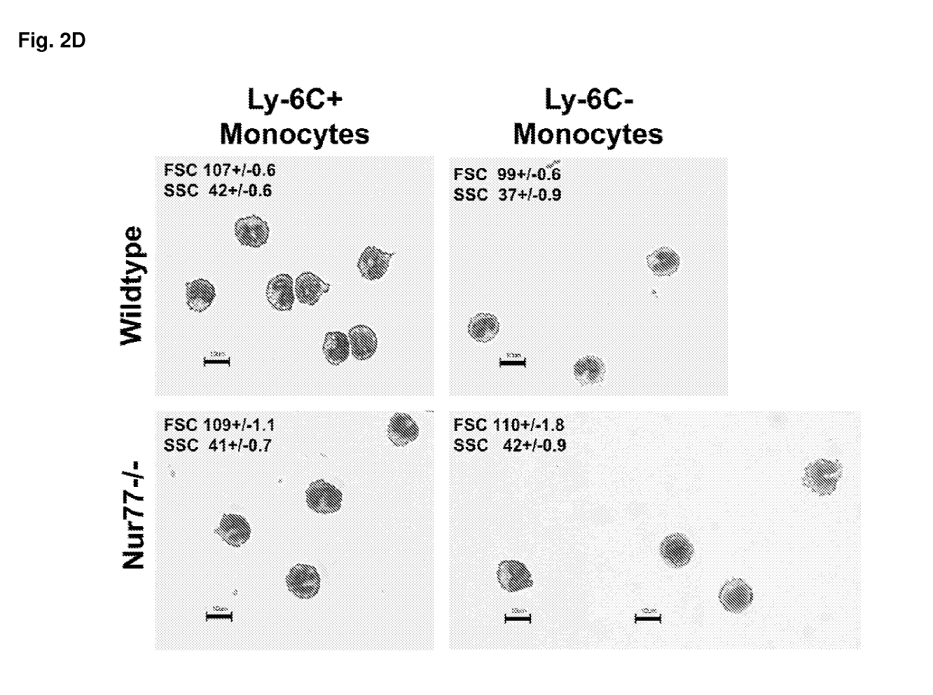

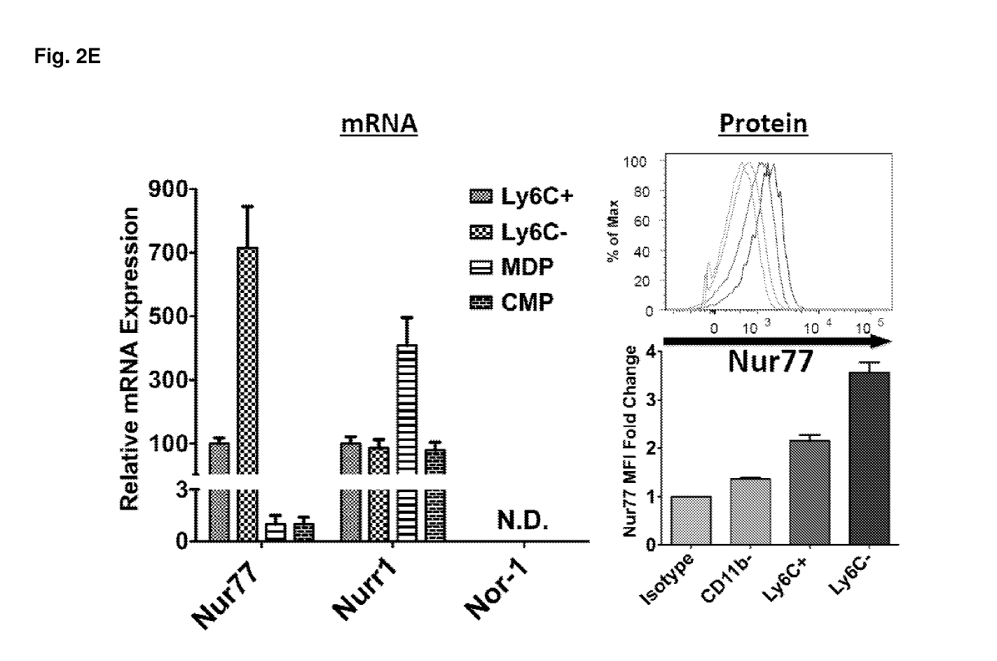

Significantly lower levels of total monocyte numbers were found in the spleen of Nur77 knockout mice compared to C57BL/6J (BL6) control mice (FIGS. 2A, 2B). The monocyte population was further examined to distinguish between Ly6C+ and Ly6C− subpopulations (FIGS. 2A, 2B). The reduction in total monocytes was attributed to a dramatic reduction in the Ly6C− monocyte population in the Nur77 knockout mice. The reduction in total Ly6C− monocyte number in the Nur77 knockout mice was comparable to the reduction in Ly6C− monocytes analyzed as a percentage of live cells by flow cytometry. Similar reductions in Ly6C− monocytes were found in the blood, and bone marrow of the global Nur77 knockout mice compared to BL6 control mice. Ly6C+ monocyte numbers were also reduced in Nur77 knockout mice, though somewhat variably and not significant. Except for a slight increase in granulocytes (<10%), there was little variation in other immune cell subsets examined (FIG. 2C). Myeloid progenitor cells including macrophage/dendritic cell precursors (MDP), the precursor to monocyte populations, were also unaltered in Nur77 knockout animals (FIG. 2C).

The specific labeling and purity of monocytes in these studies was verified using a panel of flow antibodies (FIG. 1B), and Cytospin preps of labeled cells isolated by cell sorting (FIG. 2D). Generally, Ly6C+ monocytes are larger with a more granular appearance compared to Ly6C− monocytes (Geissmann et al., Immunity 19:71 (2003); Swirski et al., Science 325:612 (2009)). Interestingly, Ly6C− monocytes that remained in the bone marrow of Nur77-deficient mice were morphologically larger, and appeared less differentiated with enlarged non-horseshoe shaped nuclei and increased granularity than their counterparts in BL6 mice. It has been hypothesized that both inflammatory and patrolling monocytes arise from MDP in the bone marrow, but specific cellular factors that mediate this differentiation are currently unknown (Woollard and Geissmann, Nat Rev Cardiol 7:77 (2010)). These findings and others indicate that Nur77 is involved in the differentiation of Ly6C− monocytes from MDP in bone marrow.

### Example 3

This example shows data indicating expression of NR4A1 family members in monocytes and myeloid progenitors.

The relative expression of Nur77 and other NR4A family members in Ly6C+ and Ly6C− monocytes, and myeloid progenitor populations in control BL6 bone marrow (FIG. 2E). Nur77 mRNA averaged 7-fold higher in Ly6C− compared to Ly6C+ monocytes. Similarly, Nur77 protein concentration was highest in Ly6C− monocyte populations, suggesting that Nur77 has a significant function in Ly6C− monocytes. Nur77 expression was also much higher in both Ly6C+ and − monocyte populations than in non-myeloid (CD11b−) cells, MDP or common myeloid precursor (CMP), implying a specific increased expression of Nur77 in monocytes, and upregulation of Nur77 with final differentiation of monocyte progenitor cells. NR4A family member Nurr1 was also expressed in monocytes and myeloid progenitor cells, while NOR-1 expression was undetectable. Interestingly, Nurr1 expression was highest in MDP cells, the precursor cell to monocytes, but was expressed at lower levels in differentiated monocyte populations.

### Example 4

This example shows that the defect in monocyte differentiation in Nur77−/− mice is MDP-derived.

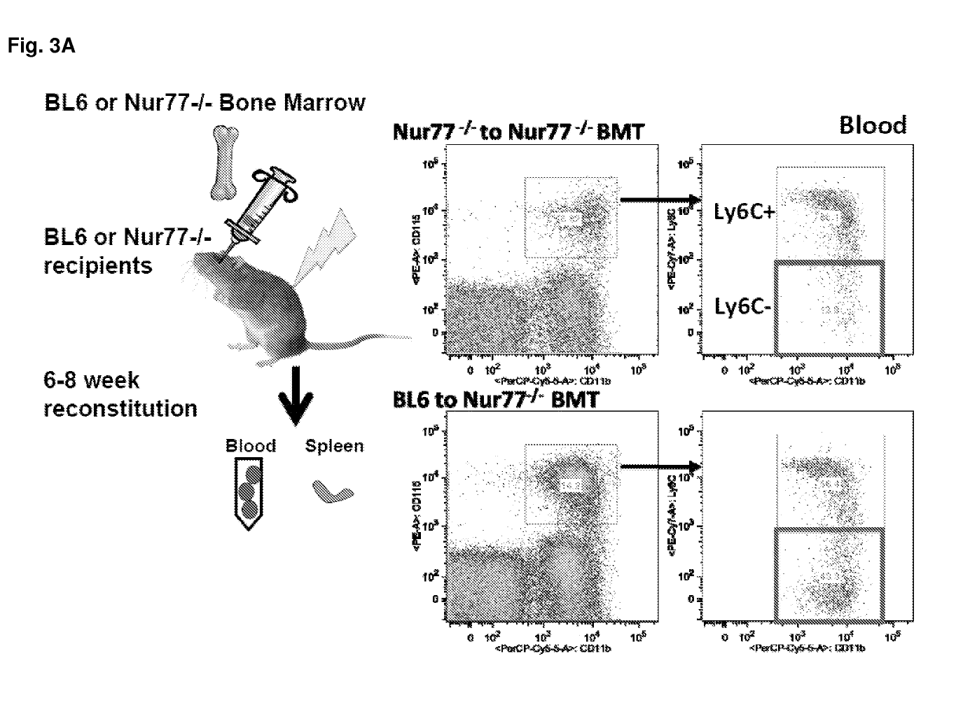

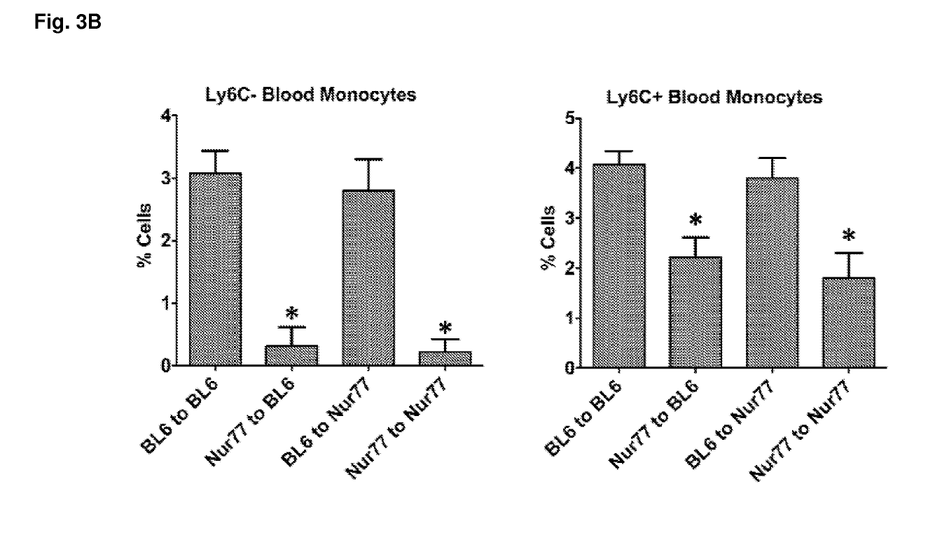

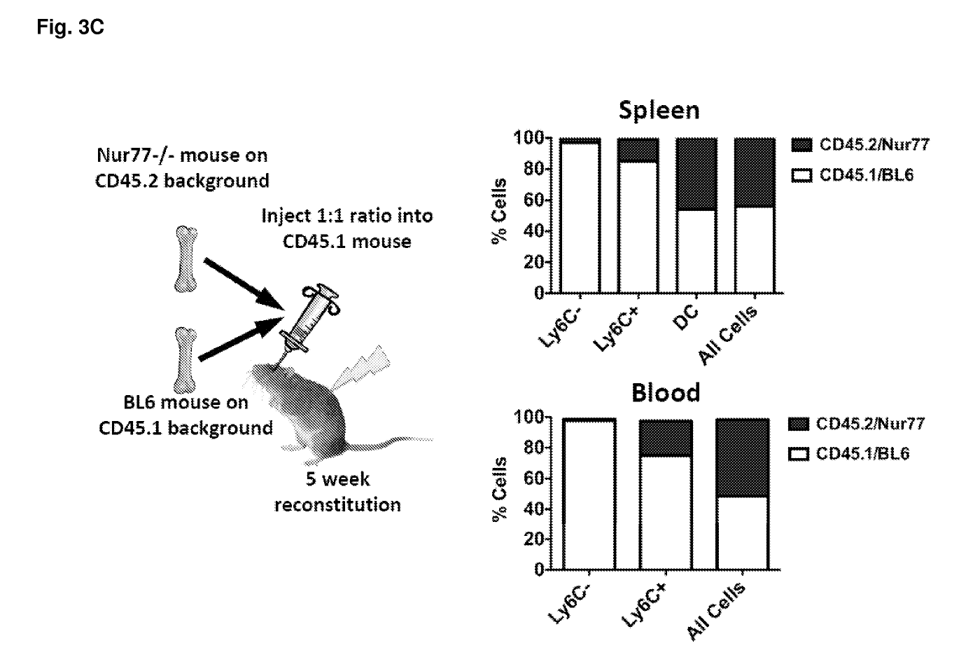

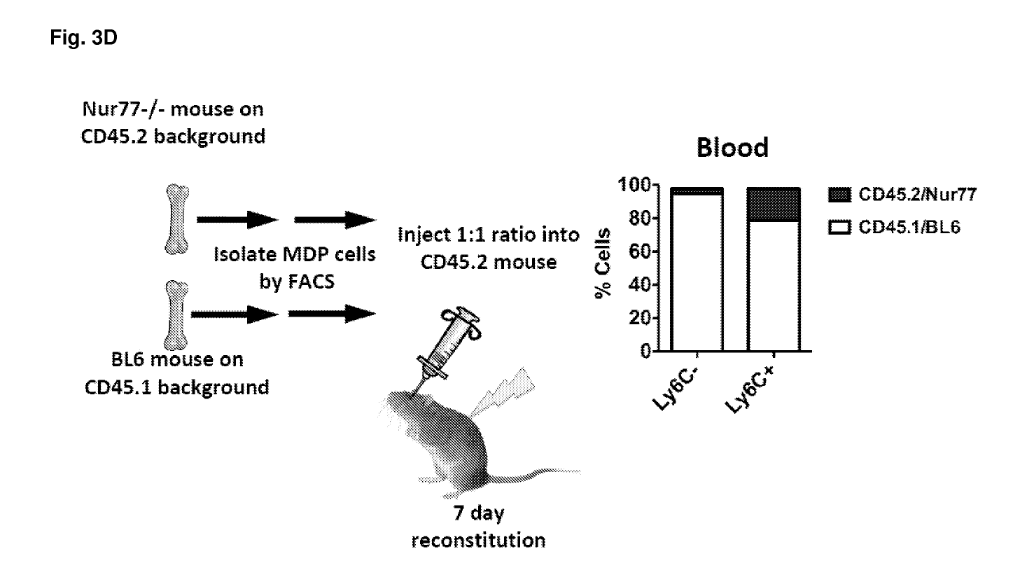

To determine if the Nur77 defect in Ly6C− monocyte differentiation is bone marrow-derived, Nur77 knockout or BL6 bone marrow was reconstituted into lethally irradiated Nur77 or BL6 recipients (FIG. 3A, 3B). Six weeks after reconstitution of Nur77 bone marrow into either BL6 or Nur77 recipients, a significant decrease in Ly6C− monocytes in blood was observed, suggesting a defect in bone marrow-derived cell differentiation. Additionally, reconstitution of BL6 bone marrow into Nur77 recipients restored the population of Ly6C− monocytes to normal levels. Ly6C+ monocytes were also significantly reduced by approximately 40% in blood of mice receiving Nur77 bone marrow.

Mixed chimera studies were performed to examine how Nur77 knockout monocytes proliferate under competitive conditions (FIG. 3C). Bone marrow from CD45.2 Nur77−/− mice and CD45.1 BL6 control mice were reconstituted 1:1 into CD45.1 recipients. Six weeks after bone marrow transfer, recipients had less than 5% Ly6C− and 25% Ly6C+ monocytes derived from Nur77 donors, while the vast majority of monocytes were derived from BL6 donors. In contrast, about equal numbers of dendritic cells (DC) and all other cells were attributed to each donor, implying no defect in Common Dendritic Precursors (CDP) or other hematopoietic stem cell populations. Nur77-deficient stem cells were specifically unable to reconstitute both Ly6C+ and Ly6C− monocyte populations, while other hematopoietic cell populations could colonize normally. These results imply a Nur77-related defect in differentiation and proliferation of both Ly6C+ and Ly6C− monocytes under reconstitution or competitive conditions. The defect is likely more pronounced in the Ly6C− monocytes due to the increased expression/function of Nur77 in Ly6C− monocytes compared to Ly6C+ monocytes.

To confirm if MDP cells directly led to the loss of monocytes in Nur77−/− mice, similar mixed chimeric studies were conducted using reconstituted MDP cells isolated by fluorescent activated cell sorting (FIG. 3D). Isolated MDP cells from CD45.2 Nur77−/− mice and CD45.1 BL6 control mice were reconstituted 1:1 into CD45.2 recipients. Seven days after bone marrow transfer, recipients had less than 10% Ly6C− and 30% Ly6C+ monocytes derived from Nur77 donors, while the vast majority of monocytes were derived from BL6 donors. These results were strikingly similar to the mixed chimera reconstitution using whole bone marrow, indicating that there is a direct defect in the production of both Ly6C+ and Ly6C− monocyte populations from Nur77 deficient MDP cells.

### Example 5

This example shows data indicating that Ly6C− monocytes die in the bone marrow of Nur77−/− mice.

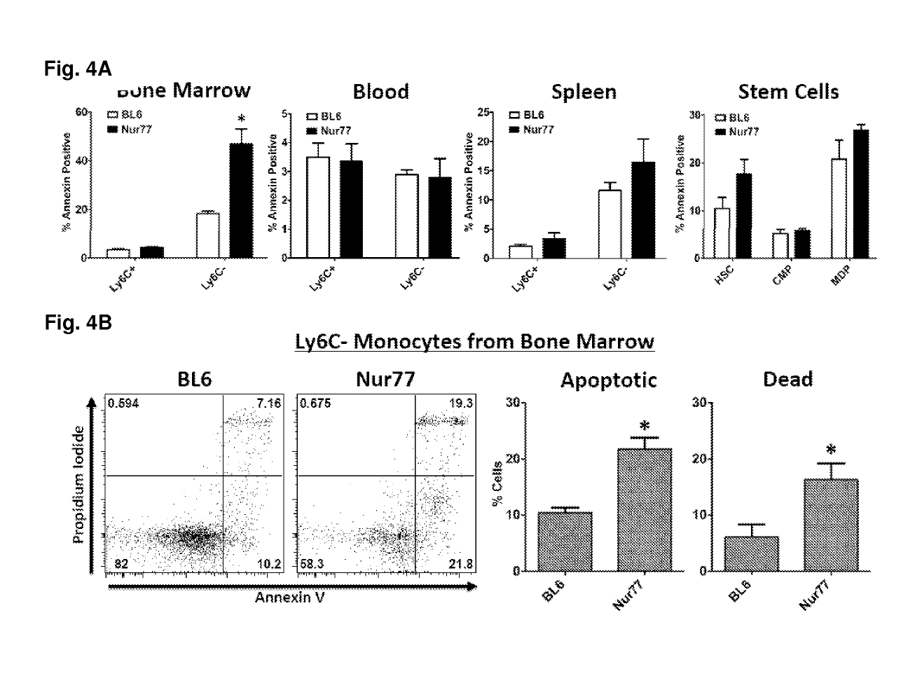

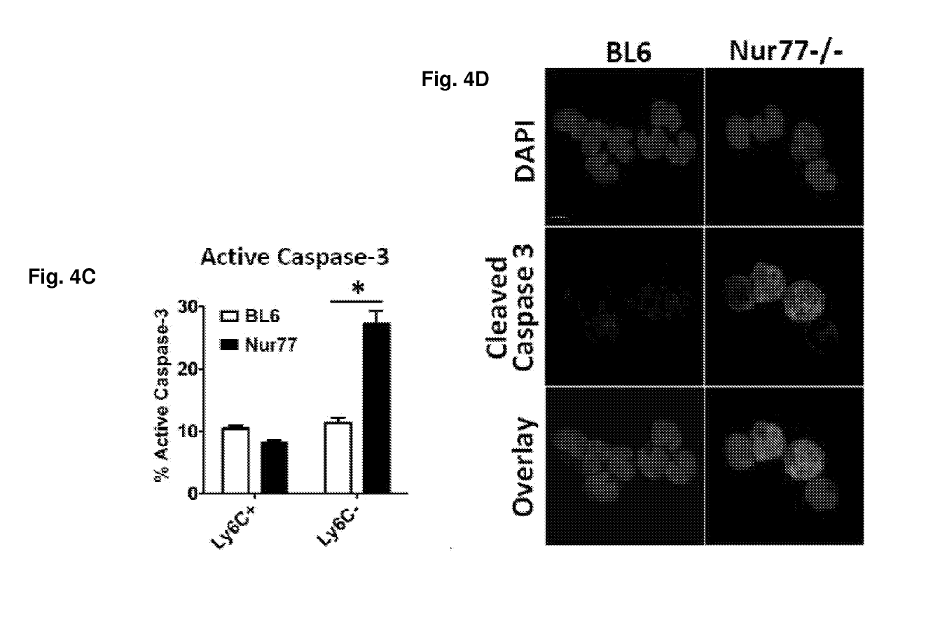

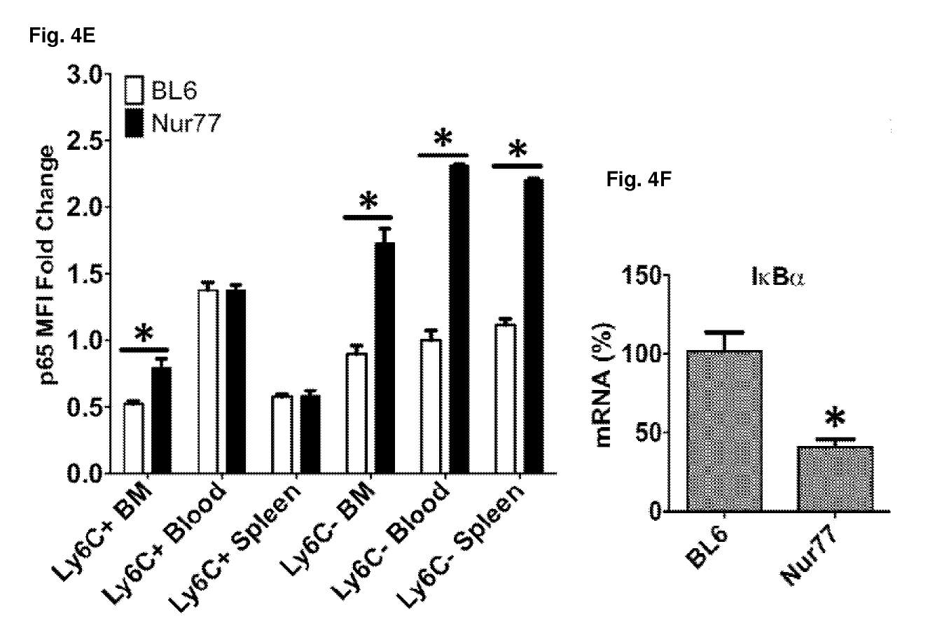

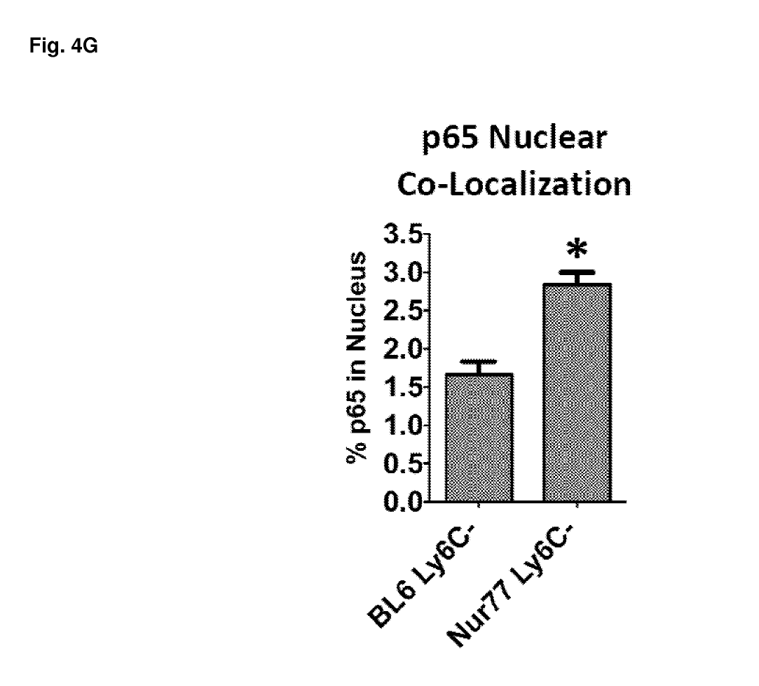

To determine the nature of the defect in monocyte production, apoptosis of Ly6C+ and Ly6C− populations in BL6 and Nur77−/− mice was first measured. Annexin V and propidium iodide staining of monocyte populations in bone marrow, blood, and spleen revealed a significant increase in the number of apoptotic and dead Ly6C− monocytes in Nur77−/− bone marrow (FIG. 4A, 4B). There were approximately twice as many apoptotic and dead Ly6C− monocytes in the Nur77−/− bone marrow compared to control. Additionally, there was higher activity of caspase 3, another hallmark of apoptosis, in Ly6C− monocytes from Nur77−/− bone marrow compared to control (FIG. 4C, 4D). There were no changes in apoptosis of Ly6C− monocytes in the blood, or spleen, suggesting that the Ly6C− monocytes are dying in the bone marrow before they egress to blood and tissues. Moreover, no changes in apoptosis in myeloid progenitor populations in Nur77−/− bone marrow, suggesting that the apoptosis occurs in the marrow during monocyte maturation were detected (FIG. 4A). Interestingly, apoptosis in Ly6C+ monocytes in bone marrow of the Nur77 knockout mouse was not observed, implying that in the steady state Nur77 expression does not regulate Ly6C+ monocyte survival (FIG. 4A, 4C).

Since Nur77 has recently been linked to regulation of the inflammatory NFKB pathway (You et al., Circ Res 104:742 (2009); Diatchenko et al., J Leukoc Biol 78:1366 (2005)), the activity of NFKB in Nur77−/− monocytes using a phospho-p65 antibody was examined (FIG. 4E). Ly6C− monocytes in the bone marrow, blood and spleen of Nur77−/− mice had significantly higher levels of p65 phosphorylation (FIG. 4E), implying increased NFKB activation and inflammatory activity in this monocyte subset. Ly6C+ monocytes from bone marrow of Nur77−/− mice also had significantly higher levels of NFKB activity (FIG. 4E). To confirm the increased activity of NFKB in Nur77−/− Ly6C− monocytes, colocization of p65 in the nucleus of Ly6C− monocytes by immunofluorescence was examined (FIG. 4G). There was an approximately two-fold increase in the amount of p65 colocalized in the nucleus of Ly6C−Nur77−/− monocytes compared to B16 control. Nur77 may suppress NFKB activity through direct induction of IKBα expression (You et al., Circ Res 104:742 (2009)), so the expression of IKBα transcript in Ly6C− monocytes from Nur77−/− bone marrow was examined (FIG. 4F). IKBα expression was reduced by more than 50% in Ly6C− monocytes from Nur77−/− mice compared to control, which may partially explain the increased NFKB activity in Nur77−/− monocytes.

### Example 6

This example describes data indicating that Ly6C− monocytes from Nur77−/− mice are arrested at S phase of the cell cycle.

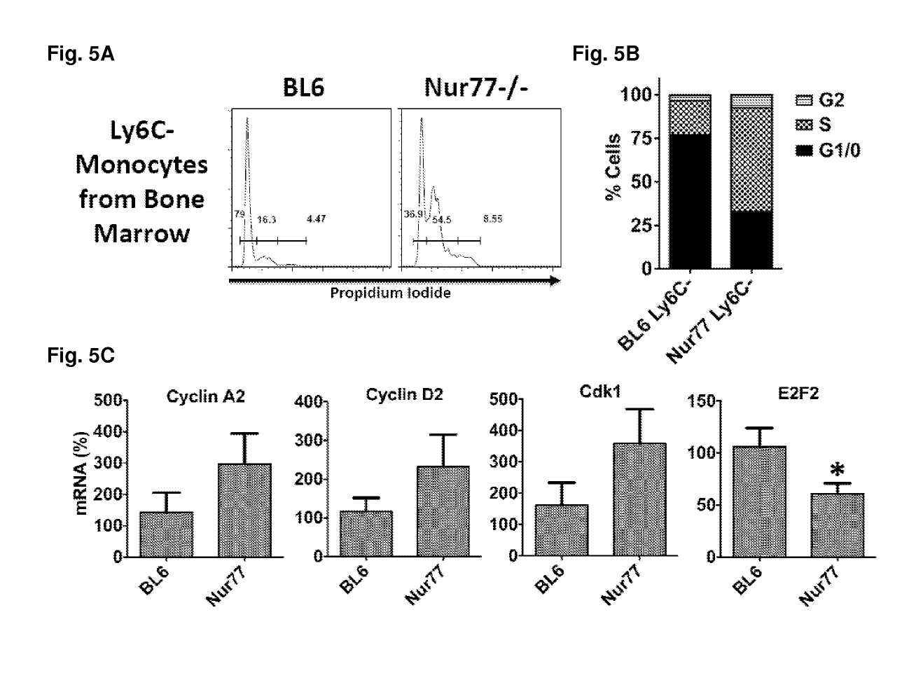

Cell cycle analysis was performed on monocyte populations in the bone marrow to determine if apoptosis occurs due to defects in cell division (FIG. 5A, 5B). Ly6C− monocytes from Nur77−/− mice had approximately three times as many cells in S phase and two times as many cells in the G2 phase of the cell cycle compared to BL6 control mice, implying a possible defect in G1 to S phase transition and acceleration of cell cycle. Transcripts of the S phase mediators Cyclin A2, Cyclin D2 and Cdk1 were all higher in Ly6C− monocytes from bone marrow of Nur77−/− compared to BL6 control mice implying an acceleration of cell cycle (FIG. 5C). Interestingly, the transcription factor E2F2, which represses S-phase entry, was expressed at significantly lower levels in Ly6C− monocytes from Nur77−/− mice. No differences in cell cycle were observed in Ly6C+ monocytes or other cells found in the bone marrow of Nur77−/− mice.

### Example 7

This example describes data indicating a reduced functionality and differentiation ability of Ly6C− monocytes of Nur77−/− mice.

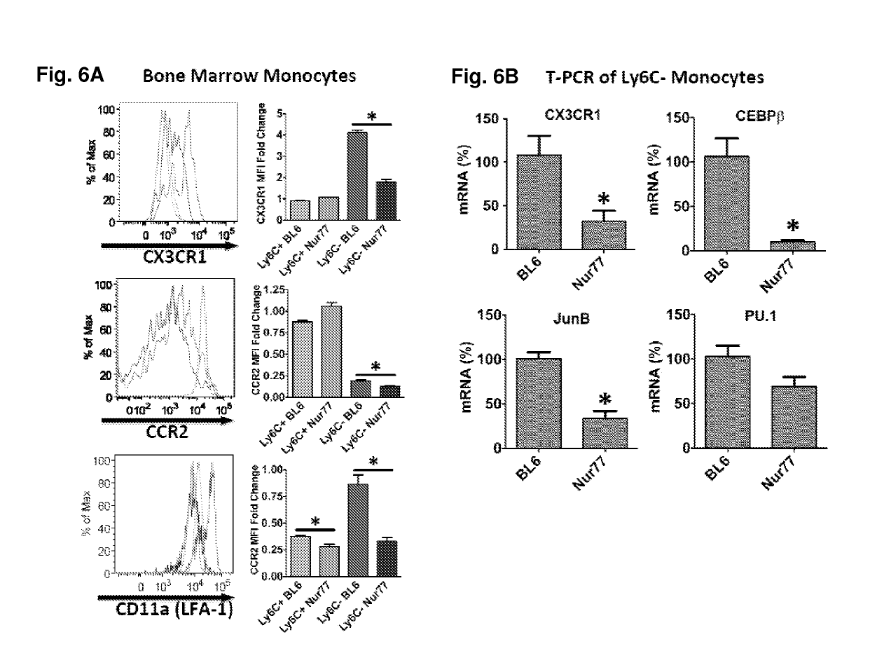

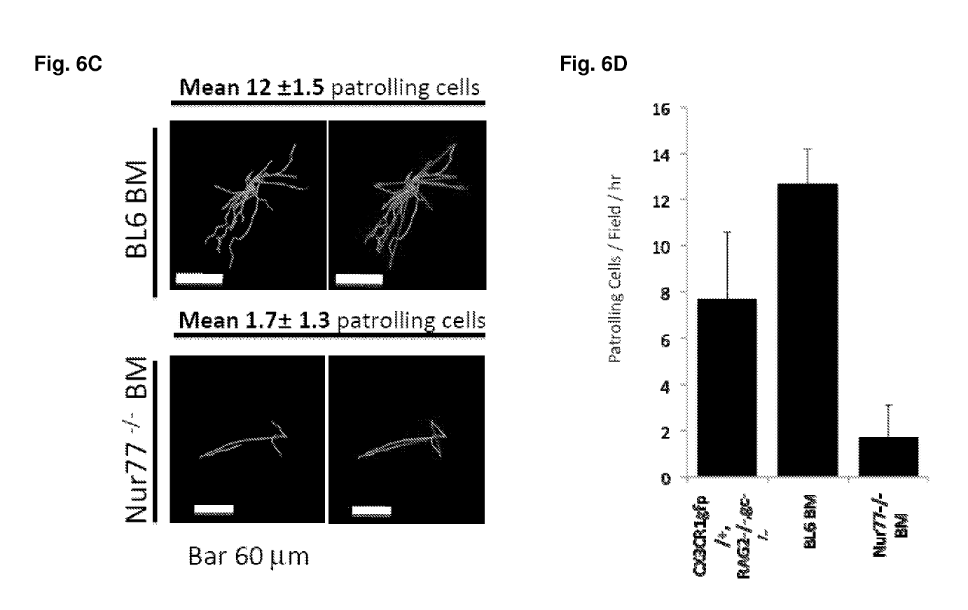

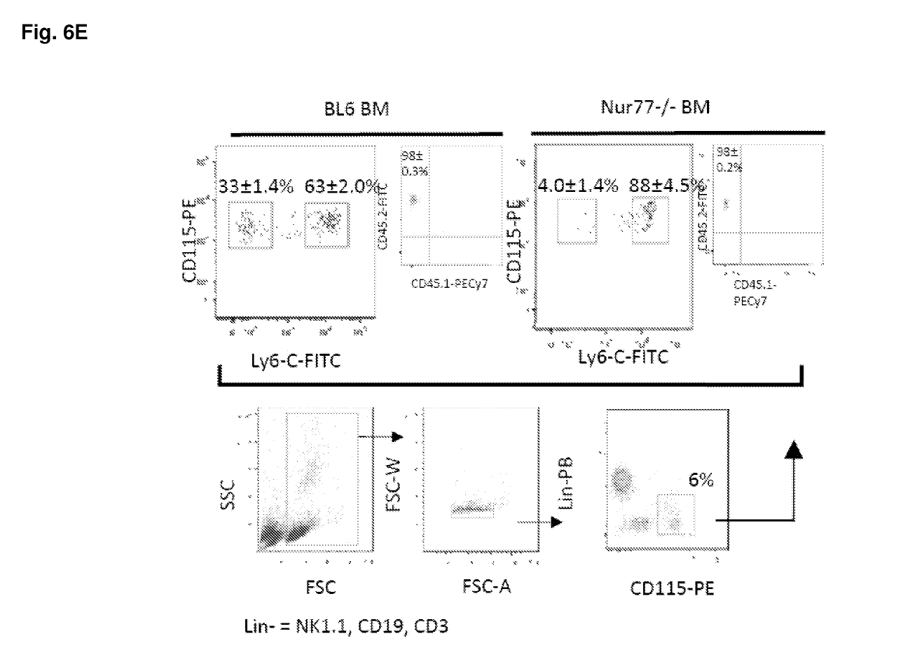

The expression of the chemokine receptors CX3CR1 and CCR2, and the adhesion molecule LFA-1, which are important for proper monocyte function in the Nur77−/− mice, were measured (FIG. 6A). The chemokine receptor CX3CR1 is normally upregulated as monocytes differentiate to Ly6C− subsets, although CX3CR1's role in monocyte physiology remains uncertain (Tacke et al., J Clin Invest 117:185 (2007)). However, Jung and colleagues have recently demonstrated that CX3CR1 is an important factor for Ly6C− monocyte survival (Landsman et al., Blood 113:963 (2009)). The absence of CX3CR1 or its ligand CX3CL1 in mice leads to a significant reduction of Ly6C− blood monocytes under steady-state and inflammatory conditions. The expression of CX3CR1 in Nur77−/− mice was less than half the normal levels found in BL6 control mice, which could potentially account for the increased apoptosis observed in Nur77−/− mice. The chemokine receptor, CCR2, is important for mediating monocyte egress and homing to tissues from the bone marrow (Boring et al., J Clin Invest 100:2552 (1997)). Ly6C− monocytes from Nur77 knockout mice expressed lower levels of CCR2 in spleen and bone marrow, ruling against the possibility that Ly6C− monocytes in Nur77 knockout mice have increased homing to tissues. Interestingly, Ly6C+ monocytes from Nur77-deficient mice had similar levels of CX3CR1 and CCR2 expression compared to BL6 control monocytes. Monocyte patrolling behavior is also dependent on the expression of the integrin LFA-1 (Auffray et al., Science 317:666 (2007)). Interestingly, LFA-1 (CD11a) expression was reduced in both Ly6C− and Ly6C+ monocytes from Nur77-deficient mice compared to BL6 control, suggesting that the Nur77-deficient monocytes lack binding ability to vessel walls.

Next, relative levels of mRNA for some factors known to be important in monocyte and macrophage differentiation was measured (FIG. 6B). Significantly lower expression of the chemokine receptor CX3CR1 (3 fold), and the transcription factors CEBPβ (10 fold) and JunB (3 fold) were observed in Ly6C− monocytes from Nur77−/− mice compared to BL6 controls, suggesting sub-par differentiation potential. Expression of the transcription factor PU.1 was not significantly different in Ly6C− monocytes from Nur77−/− mice compared to controls.

### Example 8

This example describes data indicating that patrolling cells derived from Nur77−/− bone marrow are reduced.

To determine if Nur77−/− cells could still patrol the vasculature, bone marrow from CD45.2 BL6 control or Nur77−/− mice was grafted onto lethally irradiated CD45.1 BL6 recipients, and after 6 weeks the numbers and behaviour of blood monocyte subsets were investigated by flow cytometry and intravital microscopy. BL6 bone marrow recipients had normal levels of Ly6C− and Ly6C+ subsets in the blood, whereas in Nur77−/− bone marrow recipients the Ly6C− subset was reduced by 8 fold (33±1.4% to 4±1.4%; FIG. 6E). The number of patrolling cells in the ear of Nur77−/− bone marrow recipients was reduced by 6 fold in comparison with mice receiving BL6 bone marrow (1.7±1.3/cells/flield/h vs 12±1.5; FIG. 6C) and 4 fold in comparison to CX3CR1gfp/+, RAG2−/−, gc−/− mice which lack T, B and NK cells (FIG. 6D). However, monocyte adhesion to endothelium was preserved, and the directional phenotype of patrolling was not affected, i.e. patrolling cells in both BL6 or Nur77−/− bone marrow-chimera mice could crawl against blood flow, turn 180° and traverse from side to side in vessels (FIG. 6C).

### Example 9

This example includes a description of data indicating that Gal3-BP induces Nur77 expression.

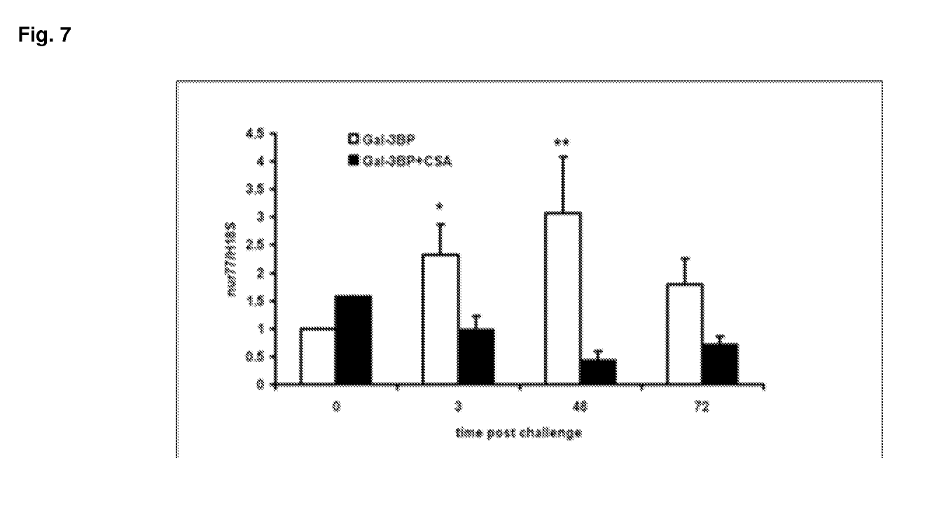

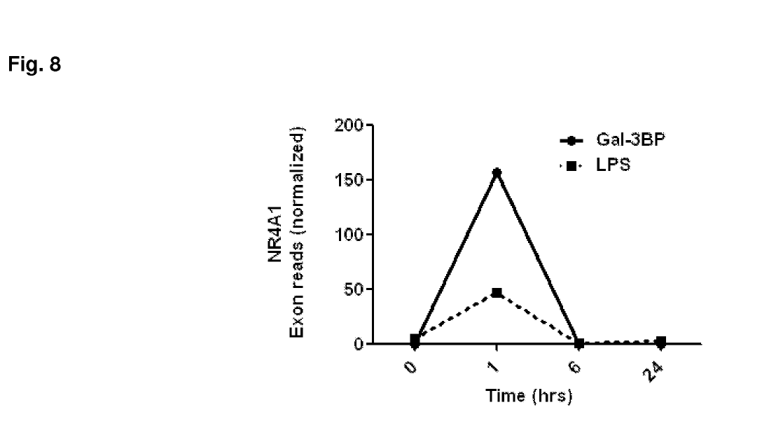

Gal3-BP induces Nur77 expression (FIG. 7) and this has anti-inflammatory effects on macrophages. This data was also verified by genome-wide screening using deep sequencing (FIG. 8). In the design of this genome-wide screening, human blood was obtained through a blood donor program. Monocytes were isolated by CD14 magnetic beads (positive selection, Stem Cell Technology human monocyte enrichment kit). The cells were plated in 6-well plates and exposed to recombinant human Gal-3BP (R&D) at 5 μg/ml or LPS (Sigma Lipopolysaccharides from Escherichia coli). Cells were harvested before and 1, 6 and 24 hours after addition of Gal-3BP or LPS. RNA was extracted by Trizol reagent combined, and up to 10 μg total RNA per well obtained. RNA was enriched for poly-A mRNA (MicroPoly(A)Purist™ Kit (Ambion, AM1919)), reverse transcribed and loaded on Illumina 1000 sequencer. Between 1.5 and 2 million exon coding reads were sequenced per sample and assembled by EDGE-R software. Read numbers were sorted by gene names and log 2 transformed. As a first screen, genes were sorted by significant expression increase (FDR<0.05) at 1 hour. To focus on the differences, the log 2 expression data in response to LPS were subtracted from those in response to Gal-3BP. Genes that were statistically (FDR<0.05) differentially expressed in response to Gal-3BP were screened, and a subset of these were at least fourfold different from the LPS-induced response.

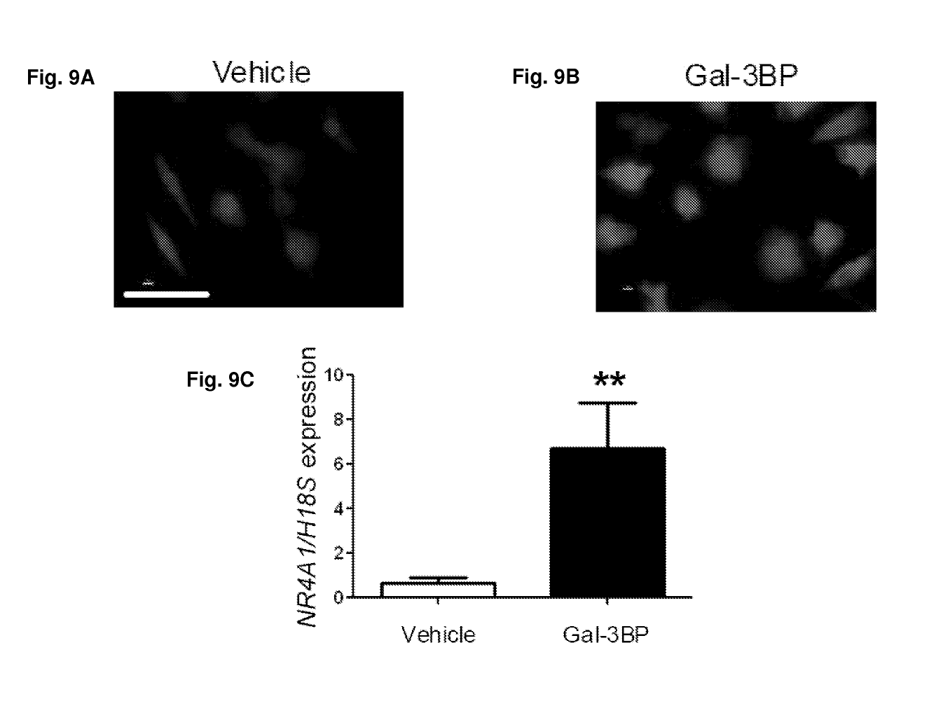

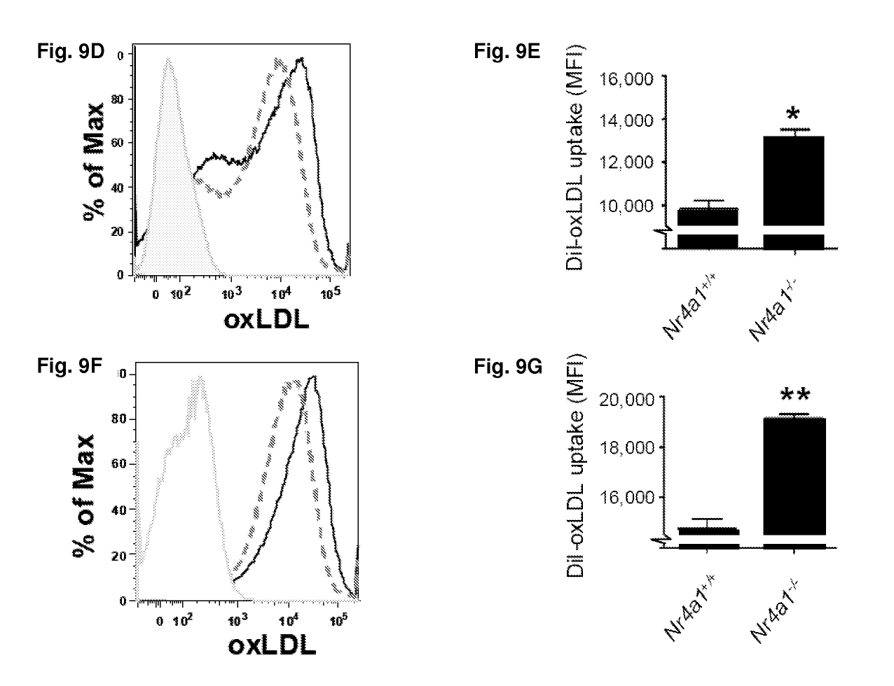

To address the mechanism by which Gal-3BP may inhibit CD36 and SR-A expression and thereby limit foam cell formation, expression of the nuclear receptor Nur77 (gene name NR4A1) in human primary macrophages was measured (FIGS. 9A-9C). Overexpression of this receptor is known to downregulate CD36 expression (Bonta, et al., Arterioscler. Thromb. Vasc. Biol. 26:2288 (2006)). NR4A1 expression was induced more than 6-fold 6 hours after Gal-3BP (FIG. 9C). At 24 hours, Nur77 protein was massively increased (FIG. 9B) compared to control (FIG. 9A).

If CD36 downregulation by Nur77 is relevant, then macrophages of Nr4a1−/− mice should show increased oxLDL uptake. When GM-CSF-differentiated bone marrow-derived cells derived from Nr4a1+/+ or Nr4a1−/− mice were exposed to oxLDL for 6 hours (FIG. 9D-9G), ungated (FIG. 9D, 9E) or CD11b+CD11c+(FIG. 9F, 9G). Nr4a1−/− cells showed significantly more ox-LDL uptake (by 30-40%) than Nr4a1+/+ cells (FIG. 9E, 9G). CD11b+CD11c+ cells include inflammatory macrophages and dendritic cells (Geissmann, et al., Science 327:656 (2010)). Taken together, these data show that Nur77 is induced by Gal-3BP and that this is relevant for regulation of oxLDL uptake.

### Example 10

This example includes a description of data showing that monocyte phenotype can determine atherosclerosis.

The effects of Nur77 on monocyte differentiation and on macrophage phenotype may be related. Although not being bound by a particular theory, one hypothesis is that Ly6C−CX3CR1 highmonocytes preferentially differentiate into non-inflammatory macrophages (Geissmann et al., Immunity 19:71 (2003)). Since Gal-3BP dramatically upregulates Nur77 expression, it is possible that Gal-3BP specifically promotes differentiation, survival or recruitment to tissues of these non-inflammatory monocytes.

The effect of manipulating Nur77 is probably both cell- and context-dependent. Additionally, although not wishing to be bound by any particular theory, signaling pathways that induce Nur77 and have a net inhibitory effect on atherosclerosis are at least partially dependent on Nur77 for this activity. Conversely, although not wishing to be bound by any particular theory, signaling pathways that have a net pro-atherogenic effect and induce Nur77 could promote more severe atherosclerosis in the absence of Nur77 function. Increased atherosclerosis in the Nur77−/− mice may be driven by the loss of Ly6C− monocytes, which might normally be protective against atherosclerosis. Alternatively, loss of Nur77 may drive production of a potently pro-inflammatory macrophage phenotype in the artery wall contributing to atherosclerosis.

To confirm support for these hypotheses, data from bone marrow transplantation studies into atherosclerosis-susceptible ldlr−/− recipient mice suggest that absence of Nur77 in myeloid cells promotes atherosclerosis (FIG. 10). Plasma lipoprotein levels were not different between the two groups. Although monocyte-macrophages are considered to be the hallmark cell type of atherosclerotic plaque initiation, Nur77−/− mice have about a 70% reduction in total monocyte numbers in blood and spleen. These data suggest that during atherogenesis, the phenotype of macrophages is as important as the quantity of macrophages present in the arterial wall.

### Example 11

This example includes a discussion of the results and conclusions based upon the results.

In this study, mice deficient in Nur77 have a defect in bone marrow production of Ly6C− monocytes. The loss of this subset in the absence of Nur77 is most likely due to an intrinsic defect in hematopoetic cell differentiation. Nur77 appears to be a critical transcription factor required for the production and homeostasis of this subset. The few Ly6C− monocytes remaining in circulation in the absence of Nur77 are morphologically similar to Ly6C+ monocytes, and have similar CX3CR1 and LFA-1 expression. Ly6C− monocytes in the Nur77 knockout appear to have drastic deficiencies in their functionality, patrolling and differentiation ability.

Higher apoptosis and cell death was found in Ly6C− monocytes within the bone marrow, implying an autophagocytic pathway related to improper differentiation or cell cycle progression. Ly6C− monocytes from Nur77−/− mice may be dying of an intrinsic apoptotic pathway due to miss-regulation of cell cycle progression in the absence of Nur77. Ly6C− monocytes from Nur77−/− mice appear stuck in S phase of cell cycle and unable to complete division. Furthermore, Nur77−/− Ly6C− monocytes appear immature implying that the population is not differentiating and proliferating properly from its yet to be identified precursor cell population. In Ly6C− monocytes from Nur77−/− mice, reduced expression of the transcription factor E2F2, which is considered the ultimate repressor for cell commitment to enter and progress through S-phase, may be partially responsible for premature entry and incompletion of the S-phase. Recent reports indicate that E2F family members are required for myeloid cell survival, and their ability to respond properly to CSF-1 signals (Trikha et al., J Biol Chem 286:4783 (2010)). However, details of how Nur77 regulates E2F2 expression leading to control of cell cycle progression in monocytes has yet to be determined. Nur77 is likely maintaining a fine balance of cell cycle control to maintain monocyte homeostasis. The closely related NR4A1 family member, Nurr1 has recently demonstrated a similar regulatory role in maintaining hematopoietic stem cell quiescence via associated upregulation of cell-cycle inhibitors (Skin et al., Nat Cell Biol 12:1213 (2010)). The findings of increased expression of Nurr1 in MDP cells, the closest identified precursor to monocyte populations, imply that Nurr1 is regulating homeostasis of monocytic and dendritic cell precursor populations. Upregulation of the cell cycle initiators Cyclin A2, Cyclin D2 and Cdk1 in Nur77−/− monocytes imply that Nur77 may have a similar role in maintaining Ly6C− and possibly Ly6C+ monocyte production.

Alternatively, Nur77 may be working as a survival/differentiation factor in Ly6C− monocytes by encouraging expression of receptors important for patrolling monocyte function including CX3CR1. However the relationship between Nur77 and the potential monocyte survival factor CX3CR1 is uncertain, as are the identities of additional factors that drive Ly6C− monocyte survival and differentiation.

A reduction of Ly6C− monocytes has been observed in the blood and spleen, but not bone marrow of CX3CR1−/− mice (Tacke et al., J Clin Invest 117:185 (2007)). This may suggest a homing issue of Ly6C− monocytes, which normally express high levels of CX3CR1, in CX3CR1−/− mice. However, the defect in monocyte production in Nur77−/− mice is likely further upstream from CX3CR1 since Ly6C− monocytes are reduced in the bone marrow of Nur77−/− mice, but not in CX3CR1-deficient mice. Nur77−/− mice have a reduction in the bone marrow implying a differentiation/apoptosis issue of Ly6C− monocyte, but do not know if Ly6C+ to Ly6C− differentiation or an intermediary between the MDP and Ly6C− monocytes is impaired.

Under conditions of bone marrow transplant, and inevitably increased inflammation, both Ly6C+ and Ly6C− monocyte subsets are reduced in the absence of Nur77. This suggests that stress or inflammatory factors may exert Nur77 mediated regulation of both Ly6C+ and Ly6C− monocyte populations. Nur77 is expressed in Ly6C+ monocytes and is likely to have a role in Ly6C+ monocyte function, though the effect of Nur77 absence is much more drastic in Ly6C− monocytes likely due to increased expression and function compared to Ly6C+ monocytes. Interestingly, activity of the NFKB pathway, an important pathway regulating inflammation, was increased in both populations of monocytes in bone marrow of Nur77−/− mice. Active Nur77 binding sites have been identified on the promoter of the NFKB inhibitory regulator, IKBα, and have been suggested as a means of down-regulating NFKB inflammatory signaling (You et al., Circ Res 104:742 (2009)). These findings indicate that in both Ly6C+ and Ly6C− monocytes, Nur77 has an important role in regulating NFKB mediated inflammatory responses, and that absence of Nur77 leads to an exaggerated inflammatory response.

Inflammation either from infection or induced by high-fat diet leads to a significant increase in the number of circulating monocytes (Serbina et al., Annu Rev Immunol 26:421 (2008); Swirski et al., J Clin Invest 117:195 (2007); Saederup et al., Circulation 117:1642 (2008)). Nur77 rapidly responds to early inflammation events and may partially drive monocyte proliferation and/or extravasation. Ly6C− monocytes have been observed to extravasate and respond rapidly (within 1 hour; Auffray et al., Science 317:666 (2007); Auffray et al., J Exp Med 206:595 (2009)), in a similar time frame as Nur77 induction in response to early inflammation (Pei et al., J Biol Chem 280:29256 (2005)). Interestingly, the corresponding Ly6C− monocyte subset in humans selectively mediates oxidized LDL uptake implying an atheroprotective role (Mosig et al., FASEB J 23:866 (2009)).

1. A method of decreasing, reducing, inhibiting, suppressing, limiting or controlling an undesirable or aberrant immune response, immune disorder, inflammatory response, inflammation autoimmune response, autoimmune disease, adverse cardiovascular event or cardiovascular disease in a subject, comprising administering a Nur77 polypeptide or subsequence thereof, a Nur77 agonist, or CD14⁺ CD16⁺ monocytes and/or CD14^{dim}CD16⁺ (CD115⁺CD11b⁺GR1⁻ (Ly6C−)) monocytes or macrophages to a subject in an amount to decrease, reduce, inhibit, suppress, limit or control the undesirable or aberrant immune response, immune disorder, inflammatory response, inflammation, autoimmune response, autoimmune disease, adverse cardiovascular event or cardiovascular disease in the subject.

2-3. (canceled)

4. The method of claim 1, wherein the adverse cardiovascular event or cardiovascular disease comprises coronary artery or heart disease, atherosclerosis, peripheral artery disease, cerebrovascular disease, renal artery disease, stroke, myocardial infarction (heart attack), ischemic heart failure, transient ischemic attack or brain trauma, or elevated blood cholesterol.

5. The method of claim 1, wherein the method decreases, reduces, inhibits, suppresses, limits or controls an adverse symptom of the undesirable or aberrant immune response, immune disorder, inflammatory response, or inflammation, an adverse symptom of the autoimmune response, disorder or disease, or an adverse symptom of the cardiovascular event or cardiovascular disease in the subject.

6. The method of claim 5, wherein the adverse symptom is swelling, pain, rash, headache, fever, nausea, diarrhea, bloat, lethargy, skeletal joint stiffness, stroke, pain, paralysis, vision impairment, other sense impairment, or tissue or cell damage.

7. The method of claim 5, wherein the adverse symptom is in an epidermal or mucosal tissue, gut, bowel, pancreas, thymus, liver, kidney, muscle, central or peripheral nerves, spleen, skin, a skeletal joint, blood vessel, or a cardio-pulmonary tissue or organ.

8. The method of claim 1, wherein the immune response, immune disorder, inflammatory response, inflammation, or autoimmune response, disorder or disease, or adverse cardiovascular event or cardiovascular disease is chronic or acute.

9. The method of claim 1, wherein the Nur77 polypeptide or subsequence thereof, Nur77 agonist, stimulates, promotes, increases or induces CD14⁺ CD16⁺ or CD14^{dim} CD16⁺ monocyte or macrophage cell production, development, survival, proliferation, differentiation or activity.

10-12. (canceled)

13. A method of stimulating, promoting, increasing or inducing CD14⁺ CD16⁺ and/or CD14^{dim} CD16⁺ monocyte or macrophage cell production, development, survival, proliferation, differentiation or activity, comprising administering a Nur77 polypeptide or a subsequence thereof, or a Nur77 agonist, to a subject in an amount that stimulates, promotes, increases or induces CD14⁺ CD16⁺ or CD14^{dim} CD16⁺ monocyte or macrophage cell production, development, survival, proliferation, differentiation or activity.

14. The method of claim 13, wherein monocyte dendritic precursor cells are contacted with a Nur77 polypeptide or a subsequence thereof, or a Nur77 agonist, in an amount that stimulates, promotes, increases or induces CD14⁺ CD16⁺ or CD14^{dim} CD16⁺ monocyte or macrophage cell production, development, survival, proliferation, differentiation or activity.

15. The method of claims 1 or 8, wherein the Nur77 polypeptide or subsequence thereof comprises a subsequence of full length Nur77 polypeptide.

16-17. (canceled)

18. The method of claims 1 or 8, wherein the Nur77 polypeptide or subsequence thereof comprises full length or a subsequence of a human protein sequence or a polymorphism thereof set forth as:
Human NR4A1 protein sequence (Homo sapiens nuclear receptor subfamily 4, group A, member 1 (NR4A1):
>NP_001189162 length = 611
MWLAKACWSIQSEMPCIQAQYGTPAPSPGPRDHLASDPLTPEFIKPTMDL
ASPEAAPAAPTALPSFSTFMDGYTGEFDTFLYQLPGTVQPCSSASSSASS
TSSSSATSPASASFKFEDFQVYGCYPGPLSGPVDEALSSSGSDYYGSPCS
APSPSTPSFQPPQLSPWDGSFGHFSPSQTYEGLRAWTEQLPKASGPPQPP
AFFSFSPPTGPSPSLAQSPLKLFPSQATHQLGEGESYSMPTAFPGLAPTS
PHLEGSGILDTPVTSTKARSGAPGGSEGRCAVCGDNASCQHYGVRTCEGC
KGFFKRTVQKNAKYICLANKDCPVDKRRRNRCQFCRFQKCLAVGMVKEVV
RTDSLKGRRGRLPSKPKQPPDASPANLLTSLVRAHLDSGPSTAKLDYSKF
QELVLPHFGKEDAGDVQQFYDLLSGSLEVIRKWAEKIPGFAELSPADQDL
LLESAFLELFILRLAYRSKPGEGKLIFCSGLVLHRLQCARGFGDWIDSIL
AFSRSLHSLLVDVPAFACLSALVLITDRHGLQEPRRVEELQNRIASCLKE
HVAAVAGEPQPASCLSRLLGKLPELRTLCTQGLQRIFYLKLEDLVPPPPI
IDKIFMDTLPF;
Or
Homo sapiens nuclear receptor subfamily 4, group A, member 1 (NR4A1)
>NP_002126 length = 598 >NP_775180 length = 598
MPCIQAQYGTPAPSPGPRDHLASDPLTPEFIKPTMDLASPEAAPAAPTAL
PSFSTFMDGYTGEFDTFLYQLPGTVQPCSSASSSASSTSSSSATSPASAS
FKFEDFQVYGCYPGPLSGPVDEALSSSGSDYYGSPCSAPSPSTPSFQPPQ
LSPWDGSFGHFSPSQTYEGLRAWTEQLPKASGPPQPPAFFSFSPPTGPSP
SLAQSPLKLFPSQATHQLGEGESYSMPTAFPGLAPTSPHLEGSGILDTPV
TSTKARSGAPGGSEGRCAVCGDNASCQHYGVRTCEGCKGFFKRTVQKNAK
YICLANKDCPVDKRRRNRCQFCRFQKCLAVGMVKEVVRTDSLKGRRGRLP
SKPKQPPDASPANLLTSLVRAHLDSGPSTAKLDYSKFQELVLPHFGKEDA
GDVQQFYDLLSGSLEVIRKWAEKIPGFAELSPADQDLLLESAFLELFILR
LAYRSKPGEGKLIFCSGLVLHRLQCARGFGDWIDSILAFSRSLHSLLVDV
PAFACLSALVLITDRHGLQEPRRVEELQNRIASCLKEHVAAVAGEPQPAS
CLSRLLGKLPELRTLCTQGLQRIFYLKLEDLVPPPPIIDKIFMDTLPF;
or a polymorphism thereof.

19. The method of claim 18, wherein the polymorphism is an amino acid substitution at one or more amino acid positions 26, 36, 74, 137, 262, or 400 of Nur77.

20. The method of claim 18, wherein the polymorphism is a change of one or more of: a (L) Leu to (V) Val at position 26; a (D) Asp to (G) Gly at position 36; a (T) Thr to (I) Ile at position 74; an (S) Ser to (L) Leu at position 137; a (G) Gly to (R) Arg at position 262; a (G) Gly to (A) Ala at position 262; or a (A) Ala to (D) Asp at position 400.

21. The method of claims 1 or 8, wherein the Nur77 polypeptide, subsequence thereof or Nur77 agonist comprises a fusion polypeptide, or a chimeric polypeptide.

22. (canceled)

23. The method of claims 1 or 8, wherein the Nur77 agonist comprises a small molecule.

24. The method of any of claims 1 or 8, wherein the Nur77 agonist or small molecule comprises one or more of: 9-cis-retinoic acid; 1-di(3-indolyl)-1-(4-X-phenyl)methanes; etoposide; 5,8-diacetoxyl-6-(1′-acetoxy-4′-methyl-3′-pentenyl)-1,4-naphthaquinones; 12-O-tetradecanoylphorbol-13-acetate; diindolylmethane analogs (C-DIMs); 6-mercaptopurine; panobinostat ((2E)-N-hydroxy-3-[4-({[2-(2-methyl-1H-indol-3-yl)ethyl]amino}methyl)phenyl]acrylamide); or octaketide Cytosporone B (Csn-B) or a derivative thereof.

25. The method of claim 24, wherein the diindolylmethane analog (C-DIM) is 1,1-bis(3′-indolyl)-1-(p-methoxyphenyl)methane (DIM-C-pPhOCH3).

26. The method of claim 24, wherein the Cytosporone B (Csn-B) derivative is n-amyl 2-[3,5-dihydroxy-2-(1-nonanoyl)phenyl]acetate or a compound having a structure set forth in Table 1.

27-33. (canceled)

34. The method of claims 1 or 13, wherein the immune disorder, inflammatory response, inflammation, autoimmune response, disorder or disease, comprises rheumatoid arthritis, juvenile rheumatoid arthritis, osteoarthritis, psoriatic arthritis, multiple sclerosis (MS), encephalomyelitis, myasthenia gravis, systemic lupus erythematosus (SLE), asthma, allergic asthma, autoimmune thyroiditis, atopic dermatitis, eczematous dermatitis, psoriasis, Sjögren's Syndrome, Crohn's disease, aphthous ulcer, iritis, conjunctivitis, keratoconjunctivitis, ulcerative colitis (UC), inflammatory bowel disease (IBD), cutaneous lupus erythematosus, scleroderma, vaginitis, proctitis, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, interstitial lung fibrosis, Hashimoto's thyroiditis, autoimmune polyglandular syndrome, insulin-dependent diabetes mellitus, insulin-resistant diabetes mellitus, immune-mediated infertility, autoimmune Addison's disease, pemphigus vulgaris, pemphigus foliaceus, dermatitis herpetiformis, autoimmune alopecia, vitiligo, autoimmune hemolytic anemia, autoimmune thrombocytopenic purpura, pernicious anemia, Guillain-Barre syndrome, stiff-man syndrome, acute rheumatic fever, sympathetic ophthalmia, Goodpasture's syndrome, systemic necrotizing vasculitis, antiphospholipid syndrome or an allergy, Behcet's disease, severe combined immunodeficiency (SCID), recombinase activating gene (RAG 1/2) deficiency, adenosine deaminase (ADA) deficiency, interleukin receptor common γ chain (γ_{c}) deficiency, Janus-associated kinase 3 (JAK3) deficiency and reticular dysgenesis; primary T cell immunodeficiency such as DiGeorge syndrome, Nude syndrome, T cell receptor deficiency, MHC class II deficiency, TAP-2 deficiency (MHC class I deficiency), ZAP70 tyrosine kinase deficiency and purine nucleotide phosphorylase (PNP) deficiency, antibody deficiencies, X-linked agammaglobulinemia (Bruton's tyrosine kinase deficiency), autosomal recessive agammaglobulinemia, Mu heavy chain deficiency, surrogate light chain (γ5/14.1) deficiency, Hyper-IgM syndrome: X-linked (CD40 ligand deficiency) or non-X-linked, Ig heavy chain gene deletion, IgA deficiency, deficiency of IgG subclasses (with or without IgA deficiency), common variable immunodeficiency (CVID), antibody deficiency with normal immunoglobulins; transient hypogammaglobulinemia of infancy, interferon γ receptor (IFNGR1, IFNGR2) deficiency, interleukin 12 or interleukin 12 receptor deficiency, immunodeficiency with thymoma, Wiskott-Aldrich syndrome (WAS protein deficiency), ataxia telangiectasia (ATM deficiency), X-linked lymphoproliferative syndrome (SH2D1A/SAP deficiency), or hyper IgE syndrome.

35. (canceled)

36. A pharmaceutical composition comprising Nur77 polypeptide or subsequence thereof, or a Nur77 agonist, or CD14⁺ CD16⁺ monocytes and/or CD14^{dim}CD16⁺ (CD115⁺ CD11b⁺GR1⁻ (LyC6−)) monocytes or macrophages and a drug or agent for treatment of an aberrant immune response, immune disorder, inflammatory response, inflammation, an autoimmune response, disorder or disease, or an adverse cardiovascular event or cardiovascular disease in a subject.
